(19) United States
(12) Patent Application Publication
(10) Pub. No.: US 2008/0241262 A1
(43) Pub. Date: Oct. 2, 2008

(54) Nanoshells and Discrete Polymer-Coated Nanoshells, Methods For Making and Using Same

(75) Inventors: T. Randall Lee, Houston, TX (US); Jun-Hyun Kim, Chicago, IL (US)

Correspondence Address: ROBERT W STROZIER, P.L.L.C, PO BOX 429, BELLAIRE, TX, 77402-0429, US

(73) Assignee: THE UNIVERSITY OF HOUSTON SYSTEM, Houston, TX (US)

(21) Appl. No.: 10/594,708

(22) PCT Filed: Mar. 29, 2005
(86) PCT No.: PCT/US2005/010528

§ 371 Date: Apr. 24, 2008

Related U.S. Application Data
(60) Provisional application No. 60/557,290, filed on Mar. 29, 2004.

(51) Int. Cl. A61K 9/14; A61P 35/00; B32B 5/16

(52) U.S. Cl. 424/490; 428/403; 428/407

(57) ABSTRACT

Nano-structures are disclosed that are ideally suited for microelectronics, medical treatment, drug-delivery systems, targeted thermal absorption media, or other similar applications, where the nano-particles include metal oxide nano-particles and metallic nano-particles including a metallic nano-shell or metallic nano-rods deposited on the surface of the nano-particles or nano-shell nano-particles including metallic nano-rods deposited on the surface of the nano-particles and where the nano-structures have a plasmon resonance. For in vivo medical applications, the plasmon resonance is tuned to a tissue-transparent frequency range. Hydrogel-coated nanostructures are also disclosed, which are capable of transitioning between a non-collapsed hydrogel and a collapsed hydrogel via thermal activation induced by electromagnetic irradiation.

### RELATED APPLICATIONS

This application claims priority PCT/JUS05/10528, filed 28 Mar. 2005, which claims priority to U.S. Provisional Patent Application 60/557,290, filed 29 Mar. 2004.

### BACKGROUND OF THE INVENTION

1. Field of the Invention

The present invention relates to the general field of nano-shells.

More particularly, the present invention relates to improved nano-particles, nano-shell nano-particles, nano-rod nano-particles and nano-rod nano-shell nano-particles allowing for greater fields of use including, but not limited, to drug-delivery applications, therapeutic applications, diagnostic applications, and electronic applications. The nano-shell nano-particles include both nanometer dielectric cores and metallic cores having deposited thereon a metallic nano-shell, a plurality of metallic nano-rods or a metallic nano-shell and a plurality of non-rods, where the nano-shell and/or the nano-rods are capable of supporting a plasmon resonance is a desired region of the electromagnetic spectrum or the nano-particle is capable of supporting a magnetic induction or other similar electromagnetic effects. The nano-particles, nano-shell nano-particles, nano-rod nano-particles and nano-rod nano-shell nano-particles can also include a nano-coating or a nano-coating including a releasable reagent. The nano-particles, nano-shell nano-particles, nano-rod nano-particles and nano-rod nano-shell nano-particles can also include a reagent associated with the surfaces thereof that is releasable upon exposure to electromagnetic radiation, an electromagnetic field, an electric field and/or a magnetic field.

2. Description of the Related Art

It is known that solid metal nano-particles (i.e., solid, single metal spheres of uniform composition and nanometer dimensions) possess unique optical properties. In particular, metal nano-particles (especially the coinage metals) display a pronounced optical resonance. This so-called plasmon resonance is due to the collective coupling of the conduction electrons in the metal sphere to the incident electromagnetic field. This resonance can be dominated by absorption or scattering depending on the radius of the nanoparticle with respect to the wavelength of the incident electromagnetic radiation. Associated with this plasmon resonance is a strong local field enhancement in the interior of the metal nanoparticle. A variety of potentially useful devices can be fabricated to take advantage of these specific optical properties.

Metal colloids have a variety of useful optical properties including a strong optical absorption and an extremely large and fast third-order nonlinear optical (NLO) polarizability. These optical properties are attributed to the phasic response of electrons in the metallic particles to electromagnetic fields. This collective electron excitation is known as plasmon resonance.

At resonance, dilute metal colloid solutions have the largest electronic NLO susceptibility of known substances. However, the utility of these solutions is limited because their plasmon resonance is confined to relatively narrow wavelength ranges and cannot readily be shifted. For example, silver particles 10 nm in diameter absorb light maximally at approximately 390 nm, while similar sized gold particles absorb maximally at about 520 nm. These absorbance maximums are insensitive to changes in particle size and various dielectric coatings on the particles.

A serious practical limitation to realizing many applications of solid metal nano-particles is the inability to position the plasmon resonance at technologically important wavelengths. For example, solid gold nano-particles of 10 nm in diameter have a plasmon resonance centered at 520 nm. This plasmon resonance cannot be controllably shifted by more than approximately 30 nanometers by varying the particle diameter or the specific embedding medium.

One method of overcoming this problem is to coat small non-conducting particles with these metals. Researchers have developed methods and materials outlining the synthesis of the composite particles having homogenous structures and defined wavelength absorbance maxima. Additional information detailing work concerning the methods of preparation of metal nano-shells can be found in U.S. Pat. Nos. 6,344,272 and 6,685,986, incorporated herein by reference. In essence nano-shell composites are particles that have two layers. One layer is immediately adjacent to and surrounds another layer. The innermost layer is said to be the core. The layer surrounding the core is said to be the shell layer. The shell is metal-like in that it can conduct electricity and is made of a metal or metal-like material. The relative thickness or depth of each particles constituent layers determines the wavelength of its absorption. Therefore, by adjusting the relative core, shell thickness, and choice of materials nano-shells may be fabricated that will absorb or scatter light at any wavelength across much of the ultraviolet (UV), visible and infrared (IR) range of the electromagnetic spectrum.

The spectral location of the maximum of the plasmon resonance peak for this geometry depends sensitively upon the ratio of the core radius to shell thickness, as well as the dielectric functions of the core and shell. The presence of a dielectric core shifts the plasmon resonance to longer wavelengths relative to a solid nanoparticle made continuously and exclusively of the metallic shell material. For a given core radius, a thin shell will have a plasmon peak that is shifted to longer wavelengths relative to a thicker shell. It is to be emphasized that metal nano-shells possess all of the same technologically viable optical properties as traditional metal nano-particles in addition to this extremely important aspect of resonance tunability.

As described in the U.S. Pat. No. 6,344,272, the nano-shells are preferably made by modifying the surface of a silica particle (the core) with aminopropyl triethoxysilane to add amine groups to the surface. These are then seeded with colloidal gold. Additional colloidal gold is added via chemical reduction in solution, to form the particle's gold shell layer. The wavelength of maximum optical absorption (λₘₐₓ) of a particle is determined by the ratio of the core radius to the shell thickness for a particle of given core and shell materials and particle diameter. Each of these variables (i.e., core radius and shell thickness) can be easily and independently controlled during fabrication of the nano-shells. Varying the shell thickness, core diameter, and the total nanoparticle diameter allows the optical properties of the nano-shells to be tuned over the visible and near-IR spectrum. By also varying the core and shell materials, which are preferably gold or silver over a silicon dioxide or Au₂S core, the tunable range can be extended to cover most of the UV to near-infrared spectrum. Thus, the optical extinction profiles of the nano-shells can be modified so that the nano-shells optimally absorb light emitted from various lasers.

With the advent of nano-shell technology, it was soon realized that tunable nano-shells would have a wide range of uses, including but not limited to energy efficient paints, windows, coatings, fabrics, vehicles, building structures, and in photo voltaic applications. The use of tunable nano-shells has also been applied to modulated drug-delivery applications.

Modulated drug-delivery allows the release profiles of therapeutic agents to be manipulated to match the physiological requirements of the patient. This type of controlled delivery system is useful for treating diseases that affect the homeostatic functions of the body, such as diabetes mellitus. Various methods of accomplishing modulated in vivo drug-delivery have been described in the literature and are currently in use or undergoing investigation. Methods involving sequestration of various therapeutic agents by a polymer matrix material have been examined. For example, U.S. Pat. No. 5,986,043, incorporated herein by reference, describes certain biodegradable hydrogels as carriers for biologically active materials such as hormones, enzymes, antibiotics, antineoplastic agents, and cell suspensions. Delivery of the sequestered drug depends on the in vivo degradation characteristics of the carrier.

Certain temperature-sensitive hydrophilic polymer gels, or hydrogels, have been described. When the temperature of the polymer is raised above its lower critical (or consolute) solution temperature (LCST), the hydrogel undergos a reversible phase transition that results in the collapse of the hydrogel structure (A. S. Hoffman et al. J. Contr. Rel. 4:213-222 (1986); and L. C. Dong et al. J. Contr. Rel. 4:223-227 (1986)). The hydrogel collapse forces soluble materials held within the hydrogel matrix to be expelled into the surrounding solution (R. Yoshida et al. J. Biomater. Sci. Polymer Edn. 6:585-598 (1994). An impediment in the development of temperature-sensitive materials into clinically useful modulated drug-delivery devices has been the lack of satisfactory means for altering the temperature of the implanted device. Ideally, the temperature change should be localized to the device to avoid damage to surrounding tissue, but the temperature change also must be rapid in order to control the conformational changes in the polymer and the drug-delivery profile. Other means of altering the temperature have been proposed and are being investigated, such as heating pads, non-targeted light and exothermic chemical reactions. Other proposed techniques for controlled drug release include the application of alternating magnetic fields to certain polymers with embedded magnetic particles to effect modulation of drug-delivery.

An available method offering a satisfactory way of obtaining localized heating to accomplish controlled, thermally-actuated drug release from implantable nano-shells while adequately avoiding potential damage to the surrounding body tissue is described in U.S. Pat. No. 6,645,517, incorporated herein by reference. The technology allows for nano-shells as employers of heat-transfer agents that are embedded within a hydrogel polymer matrix. As the near-IR light is absorbed by the nano-shells, heat is generated and transferred to the polymer matrix nearby. As a result, the temperature of the polymer is increased above the polymer's lower critical solution temperature (LCST), causing a conformational change in the copolymer that leads to alterations in the release profile of the entrapped drug.

Although nano-shells and their uses have been developed, there is still a need in the art for improved nano-shells having reduced particle size, reduced and more uniform nano-shell thicknesses, enhanced and/or unique optical properties, and active coating for use in applications such as micro- and nano-scale electronics and medical applications.

### DEFINITIONS USED IN THE INVENTION

The term “nanometer” is 10⁻⁹ meter and is abbreviation “nm.”

The term “nano-particle” is defined as a particle having dimensions of from 1 to 5000 nanometers, having any size, shape or morphology. For example, they may be metal colloids such as gold colloid or silver colloid. The nanoparticles may be fullerenes which are available in both nanosphere and nanotube structures.

The term “nano-shell” means a shell having a thickness of less than 1 micron deposited or formed on a nano-particle.

The term “nano-shell nano-particle” means a nano-particle having formed thereon a partial or complete nano-shell. The term nano-shell and nano-shell nano-particle can and are sometimes used interchangeably.

The term “nano-rod” means a rod having a dimensions (length, width and height) all less than 1 micron.

The term “nano-rod nano-particle” means a nano-particle having formed thereon a nano-rod, generally a plurality of nano-rods.

The term “nano-rod nano-shell nano-particle” means a nano-particle having formed thereon a nano-shell which has formed thereon a nano-rod, generally a plurality of nano-rods.

The term “a” or “an” may mean one or more and when used in conjunction with the word “comprising”, the words “a” or “an” may mean one or more than one.

The term “another” may mean at least a second or more.

The term “non-tissue” is defined as any material that is not human or animal tissue.

The terms “cell,” “cell line,” and “cell culture” as used herein may be used interchangeably. All of these terms also include their progeny, which are any and all subsequent generations. It is understood that all progeny may not be identical due to deliberate or inadvertent mutations.

The term “targeted” as used herein encompasses the use of antigen-antibody binding, ligand-receptor binding, and other chemical binding interactions, as well as non-chemical means such as direct injection.

The term “energy source” encompasses any and all forms of excitation, including radiation from any or all regions of the electromagnetic spectrum, ultrasound, magnetic fields, electric fields, microwave radiation, laser excitation, etc.

The term “light” means electromagnetic radiation.

The term “electromagnetic radiation” is defined as radiation having an electric field and a magnetic field propagating at right angles to one another and is further limited to only the following: microwaves, infrared, visible, ultraviolet, x-rays, gamma rays, and cosmic rays. As used herein, “electromagnetic radiation” does not include radio-frequency radiation.

The term “non-cellular non-tissue material” is any biological material other than cells and tissue and may include plaque, virus material, etc.

The term “delivering” nanoparticles to a location is defined as effecting the placement of the nanoparticles attached to, next to, or sufficiently close to the location such that any heat generated by the nanoparticles is transferred to the location and any imaging of the local environment by the nanoparticles includes imaging of the desired location.

The term “illuminate” is defined as shedding electromagnetic radiation or other energy sources in such a way as to resolve or to otherwise differentiate an object from adjacent objects or to resolve distinct regions within one object.

The term “nanoparticle” is defined as a particle having a diameter of from 1 to 1000 nanometers, having any size, shape or morphology. As used herein, “nanoshell” is a nanoparticle having a discrete dielectric or semiconducting core section surrounded by one or more conducting shell layers. A “nanoshell” is a subspecies of nanoparticles characterized by the discrete core/shell structure. Both nanoshells and nanoparticles may contain dopants such as Pr⁺³, Er⁺³, and Nd⁺³.

The term “nanoparticle” means one or more nanoparticles. As used herein, “nanoshell” means one or more nanoshells. As used herein, “shell” means one or more shells.

The term “tumor” as used herein includes any swelling or tumefaction. As used herein, tumor also refers to a neoplasm.

The term “benign tumor” as used herein is defined as a tumor does not form metastases and does not invade or destroy adjacent tissue. The term “malignant tumor” as used herein is defined as a tumor that invades surrounding tissues, is usually capable of producing metastases, may recur after attempted removal.

The term “cancer” as used herein is defined as a general variety of malignant neoplasms. Cancer herein is interchangeable with carcinoma and sarcoma.

The term “antibody” as used herein, refers to an immunoglobulin molecule, which is able to specifically bind to a specific epitope on an antigen. As used herein, an antibody is intended to refer broadly to any immunologic binding agent such as IgG, IgM, IgA, IgD and IgE. Antibodies can be intact immunoglobulins derived from natural sources or from recombinant sources and can be immunoactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1988; Houston et al., 1988; Bird et al., 1988).

The term “coupling” refers to any chemical association and includes both covalent and non-covalent interactions.

The term “autoimmune disease” as used herein is defined as a disorder that results from autoimmune responses. Autoimmunity is an inappropriate and excessive response to self-antigens. Examples include but are not limited to, Addision's disease, Graves' disease, multiple sclerosis, myxedema, pernicious anemia, rheumatic fever, rheumatoid arthritis, systemic lupus erythematosus, and ulcerative colitis.

The term “inflammation” as used herein, is a general term for the local accumulation of fluid, plasma proteins, and white blood cells that is initiated by physical injury, infection or a local immune response. This is also known as an inflammatory response. The cells that invade tissue undergoing inflammatory responses are often called inflammatory cells or an inflammatory infiltrate.

The term “IR” means infrared.

The term “UV” means ultraviolet.

The term “VIS” means visible.

The term “localized” means substantially limited to a desired area with only minimal, if any, dissemination outside of such area.

### SUMMARY OF THE INVENTION

### Nano-Shell, Nano-Rod, and Nano-Rod, Nano-Shell Nano-Particles

The present invention provides unique and/or improved nano-shell nano-particles comprising a nano-particle core and a nano-shell deposited thereon, where the core is a metal or oxide nano-particle and where the nano-shell is a noble metal nano-shell or a noble metal-containing alloy nano-shell, and/or where the nano-shells are more uniform and thinner and have similar or improved optical, electro-magnetic, electrical and/or magnetic properties.

The present invention also provides nano-shell nano-particles including a core and a nano-shell formed thereon, where the core is comprised of a metal or alloy and the nano-shell is comprised of a metal or alloy in which the core and shell metals or alloys are the same or different.

The present invention also provides metallic nano-shell nano-particles including a metal nano-particle core and a metal nano-shell deposited thereon, where the core metal is preferably a noble metal, a ferromagnetic metal, a magnetic metal or an alloy or oxide thereof including any combinations and mixtures thereof and where the metal nano-shell is a metal or a metal alloy and where the nano-shell nano-particles have a plasmon resonance. Generally, the metal-metal nano-shell nano-particles have a smaller particle size than their silica-core counterparts. The smaller particle size gives these metal-metal nano-shell nano-particles enhanced properties, such as improved optical properties for use in optical electronics (e.g., OLED displays), diagnostic imaging, explosives detonation, and improved properties for used in drug-delivery systems for treating cancer and other diseases. These metal-metal nano-shell nano-particles have optical properties ideally suited for electrooptical devices, drug-delivery systems, and systems designed for the thermal killing of cells at designated body sites.

The present invention also provides dielectric (preferably oxide or sulfide), metal, or metal oxide core nano-particles having formed on an outer surface thereof nano-rods, where the nano-rods are noble metal or noble metal alloy nano-rods and where the nano-rods are grown on or from the outer surface assuming uniform and/or non-uniform sizes and/or directions and/or orientations on the outer surface providing for a different format for achieving light-to-heat energy transfer (i.e., the plasmon resonance of the nano-rod nano-particles may be distinct from that of the plasmon resonance of nano-shell nano-particles). These nano-rod nano-particles have optical properties ideally suited for electrooptical devices, drug-delivery systems, and systems designed for thermally killing cells at designated body sites.

The present invention also provides dielectric (preferably oxide or sulfide), metal, or metal oxide core nano-particles having formed on an outer surface thereof a nano-shell, which in turn has formed thereon nano-rods, where the nano-shell and the nano-rods comprise a noble metal or noble metal alloy, where the nano-rods are grown on or from the outer surface of the nano-shell assuming uniform and/or non-uniform sizes and/or directions and/or orientations on the outer surface of the nano-shell providing for a different format for achieving light-to-heat energy transfer (i.e., the plasmon resonance of the nano-shell nano-particle substructure, the nano-rod nano-shell substructure and the nano-rod nano-shell nano-particle structure may be distinct from the plasmon resonance in traditional nano-shell nano-particles). These nano-rod nano-shell nano-particles have optical properties ideally suited for electrooptical devices, drug-delivery systems, and systems designed for thermally killing cells at designated body sites.

The present invention also provides a polymer nano-particle having therein a metal nano-particle, where the polymer is preferably a hydrogel and the metal is preferably a noble metal.

### Layered Structures

The present invention also provides an electronic composite structure including at least one layer comprising nano-constructs of this invention (any of the above described nano-particles), where the layer produces a desired electrical, magnetic or optical property.

The present invention also provides an electronic composite structure including at least one layer comprising nano-constructs of this invention (any of the above described nano-particles), where the layer is part of an OLED display.

The present invention also provides an electronic composite structure including at least one layer comprising nano-constructs of this invention (any of the above described nano-particles), where the layer is a conductive layer.

The present invention also provides an electronic composite structure including at least one patterned layer comprising regions loaded with nano-constructs of this invention (any of the above described nano-particles) and non-filled regions, where the nano-construct regions are conductive and the non-filled regions are non-conductive.

The present invention also provides a multi-layer electronic composite structure including at least one patterned layer comprising regions loaded with nano-constructs of this invention (any of the above described nano-particles) and non-filled regions, where the nano-construct regions are conductive and the non-filled regions are non-conductive.

### Polymer-Coated Nano-Particles

The present invention also provides polymer-coated nano-particles comprising nano-particles of this invention (any of the above described nano-particles) formed thereon a bio-compatible polymer coating, where the polymer-coating either releases or changes size upon exposure of the nano-particles to a source of energy that is converted to heat, where the energy source can be a plasmon resonance, electromagnetic, electric or magnetic inductive heating.

### Drug-Delivery Systems

The present invention also provides a drug-delivery system comprising a nano-construct of this invention (any of the above described nano-particles) having absorbed on its surface a pharmaceutically active material that is released upon exposure of the nano-particle to a source of energy that is converted to heat, where the energy source can be a plasmon resonance, electromagnetic, electric or magnetic inductive heating.

The present invention also provides a drug-delivery system comprising a nano-particles of this invention (any of the above described nano-particles) having a bio-compatible polymer-coated thereon and impregnated with at least one pharmaceutically active agent, where the nano-particles have a plasmon resonance in a tissue-transparent window of the electro-magnetic spectrum such as the near-IR spectral window. The plasmon resonance is tuned by controlling the diameter of the nano-particle and the thickness of the nano-shell or the dimensions of the nano-rods deposited or formed on the surface of the nano-particles. For shells, larger diameters and smaller shell thicknesses shift the plasmon resonance to longer wavelengths or lower frequencies. The polymer coatings are capable of either collapsing, decomposing, or changing thickness upon heating so that the pharmaceutically active agent can be released.

The present invention also provides a drug-delivery system comprising a nano-construct of this invention (any of the above described cano-particles) having a bio-compatible hydrogel formed thereon and impregnated with at least one pharmaceutically active agent (preferably an effective amount of the at least on pharmaceutically active agent), where the nano-particles have a plasmon resonance in a tissue-transparent window of the electro-magnetic spectrum such the near-IR spectral window. The plasmon resonance is tuned by controlling the diameter of the nano-particle and the thickness of the nano-shell or the dimensions of the nano-rods deposited or formed on the surface of the nano-particles. For shells, larger diameters and smaller shell thicknesses shift the plasmon resonance to longer wavelengths or lower frequencies. The hydrogel are capable of changing thickness upon heating so that the hydrogel can be impregnated with a pharmaceutically active material when in its expanded state and warmed to transition the hydrogel from its expanded state to its collapsed state releasing the pharmaceutically active material.

The present invention also provides a layer including a bio-compatible polymer having dispersed therein nano-particles of this invention (any of the above described nano-particles) and a pharmaceutically active agent, where the pharmaceutically active agent is released upon exposure of the layer to a source of energy that is converted to heat, where the energy source can be a plasmon resonance, electric or magnetic inductive heating.

The present invention also provides a layer including a bio-compatible polymer having dispersed therein nano-particles of this invention (any of the above described nano-particles) and a pharmaceutically active agent, where the pharmaceutically active agent is released upon exposure of the layer to a source of energy that is converted to heat, where the energy source can be a plasmon resonance, electric or magnetic inductive heating.

### Other Compositions

The present invention also provides a light or temperature driven volume or size oscillator comprising a nano-particle of this invention (any of the above described compositions) having a hydrogel coating, where the nano-particles have a plasmon resonance and where periodic or cyclic heating and cooling or irradiating and non-irradiating causes the hydrogel coated nano-construct to oscillate between a first size or volume and a second size or volume.

The present invention also provides a light or temperature driven valve comprising a polymer-coated nano-particle of this invention (any of the above described compositions) formed into a thin layer so that when the nano-particles are exposed to light causing the polymer coating to collapse allowing material to flow through the layer, where the nano-particles have a plasmon resonance and where periodic or cyclic heating and cooling or irradiating and non-irradiating causes the hydrogel coated nano-construct to oscillate between a first size or volume and a second size of volume.

The present invention also provides an explosive including an explosive and an effective amount of a nano-particle of this invention, where the effective amount is sufficient to detonate the explosive, when the nano-particles are exposed to a source of energy that it is able to liberate heat.

### Method for Making and Using

The present invention also provides a method for making the above described nano-shell nano-particles including the step of growing a noble metal or noble metal alloy nano-shell on the outer surface of metal, metal alloy, and/or dielectric nano-particles, where the resulting nano-shell nano-particles have improved structure, size, optical, electro-magnetic, electrical and/or magnetic properties.

The present invention also provides a method for making the above described nano-rod nano-particles including the step of growing noble metal or noble metal alloy nano-rods on the outer surface of metal, metal alloy, and/or dielectric nano-particles, where the resulting nano-rod nano-particles have improved structure, size, optical, electro-magnetic, electrical and/or magnetic properties.

The present invention also provides a method for making the above described nano-rod nano-shell nano-particles including the step of growing noble metal or noble metal alloy nano-rods on the outer surface of metal or metal alloy nano-shell, metal, metal alloy, and/or dielectric nano-particles, where the resulting nano-rod nano-shell nano-particles have improved structure, size, optical, electro-magnetic, electrical and/or magnetic properties.

The present invention also relates to a method for preparing bio-compatible polymer-coated, such as bio-compatible hydrogel-coated, nano-shell nano-particles or bio-compatible polymer-coated nano-rod nano-particles or nano-rod nano-shell nano-particles including the steps of contacting nano-particles, nano-shell nano-particles, nano-rod nano-particles, and/or nano-rod nano-shell nano-particles of this invention with a modifier including a metal reactive group or moiety and a polymer-initiator group or moiety or monomer, such as a hydrogel-initiator group or moiety or a monomer. During or after the nano-particles, nano-shell nano-particles, nano-rod nano-particles, and/or nano-rod nano-shell nano-particles of this invention are reacted with the modifier, a polymer, such as a hydrogel, is grown on the nano-p articles, nano-shell nano-particles, nano-rod nano-particles, and/or nano-rod nano-shell nano-particles of this invention forming polymer-coated, such as hydrogel-coated, nano-particles, nano-shell nano-particles, nano-rod nano-particles, and/or nano-rod nano-shell nano-particles of this invention. Alternatively, the method includes the steps of contacting the nano-particles, nano-shell nano-particles, nano-rod nano-particles, and/or nano-rod nano-shell nano-particles of this invention with a polymer-templating agent, such as a hydrogel-templating agent, to form templated nano-particles, nano-shell nano-particles, nano-rod nano-particles, and/or nano-rod nano-shell nano-particles of this invention, which are contacted with a polymer-forming solution, such as a hydrogel-forming solution, to generate bio-compatible polymer-coated, such as a hydrogel-coated, nano-particles, nano-shell nano-particles, nano-rod nano-particles, and/or nano-rod nano-shell nano-particles of this invention.

The present invention also relates to a method for preparing a drug-delivery system including the step of contacting bio-compatible polymer-coated, such as a hydrogel-coated, nano-particles, nano-shell nano-particles, nano-rod nano-particles, and/or nano-rod nano-shell nano-particles of this invention with a pharmaceutically active compound under conditions to cause impregnation of the pharmaceutically active compound into the polymer coating, such as the hydrogel coating.

The present invention also relates to a method for delivering a pharmaceutical compound to a tissue site including the step of administering polymer-coated, such as a hydrogel-coated, nano-particles, nano-shell nano-particles, nano-rod nano-particles, and/or nano-rod nano-shell nano-particles of this invention impregnated with an effective amount of the pharmaceutically active compound and then exposing the tissue site to light corresponding to the plasmon resonance of the nano-particles causing the polymer such as the hydrogel to change structure, such as collapse, releasing the pharmaceutically active compound.

For applications involving the use of polymer-coated (such as hydrogel-coated) nano-particles of this invention for thermally activated delivery of a given material or thermally activated absorption of a material, the nano-particles of this invention have a plasmon resonance in a tissue-transparent frequency of electromagnetic spectrum (e.g., the near IR or others), the nano-shell nano-particles of this invention have a plasmon resonance in a tissue-transparent frequency of electromagnetic light (e.g., the near IR or others), the nano-rod nano-particles of this invention have a plasmon resonance in a tissue-transparent frequency of electromagnetic light (e.g., the near-IR or others), and nano-rod nano-shell nano-particles of this invention have a plasmon resonance in a tissue-transparent frequency of electromagnetic light (e.g., the near-IR or others).

The present invention also relates to a drug-delivery system, including nano-particles, nano-shell nano-particles, nano-rod nano-particles, and/or nano-rod nano-shell nano-particles of this invention, having associated therewith body or tissue site specific antibodies or other agents including external magnetic fields capable ofdirectly or concentrating the nano-particles, nano-shell nano-particles, nano-rod nano-particles, and/or nano-rod nano-shell nano-particles of this invention in a desired body or tissue site. Once the nano-particles, nano-shell nano-particles, nano-rod nano-particles, and/or nano-rod nano-shell nano-particles of this invention are present at the body or tissue site, the body or tissue site is irradiated resulting in: (a) releasing site specific pharmaceuticals in or at the body or tissue site, (b) thermally killing cells or foreign organisms in or at the body or tissue site, (c) releasing enzyme inhibitors or enzyme activators in or at the body or tissue site, (d) releasing antigens to invoke an immune response in or at the body or tissue site, (d) absorption of bodily fluids, enzymes, proteins, poisons, metals, etc. in or at the body or tissue site, or (e) suppression or stimulation of other biological processes in or at the body or tissue site.

The present invention also relates to a method for delivery a drug-delivery system including nano-particles, nano-shell nano-particles, nano-rod nano-particles, and/or nano-rod nano-shell nano-particles of this invention having absorbed to their surfaces an effective amount of a pharmaceutically active agent, where the method comprises administering the drug-delivery system to an animal including a human, where the administration can be via intravenous (i.v.) administration, via intra-arterial administration, or via direct injection into a tissue site. Once a sufficient concentration of the drug-delivery system has accumulated in a target tissue site such as a tumor, other cancer sites, disease site or other site to which drug and thermal treatment is desired, exposing the tissue site to an intensity of light in a region of the electromagnetic spectra where the nano-particles have a sufficient extinction coefficient of their plasmon resonance to thermalize the light into heat releasing the absorbed agent, where the agent is adapted to treat the site either by killing cancer cells or disease cells. Concurrent or after the nano-particles have been warmed sufficiently to release the agent, changing the intensity of the light so that the nano-particles become hotter increasing a kill efficacy of the drug-delivery system.

The present invention also relates to a method for delivery a drug-delivery system including polymer-coated nano-particles, nano-shell nano-particles, nano-rod nano-particles, and/or nano-rod nano-shell nano-particles of this invention having impregnated in the polymer coating an effective amount of a pharmaceutically active agent, where the method comprises administering the drug-delivery system to an animal including a human, where the administration can be via intravenous (i.v.) administration, via intra-arterial administration, or via direct injection into a tissue site. Once a sufficient concentration of the drug-delivery system has accumulated in a target tissue site such as a tumor site, exposing the tissue site to an intensity of light in a region of the electromagnetic spectra where the nano-particles have a sufficient extinction coefficient of their plasmon resonance to thermalize the light into heat either to collapse the polymer releasing the agent or releasing the polymer coating including the agent, where the agent is adapted to treat the site either by killing cancer cells or disease cells. Concurrent or after the nano-particles have been warmed sufficiently to release the agent, changing the intensity of the light so that the nano-particles become hotter increasing a kill efficacy of the drug-delivery system.

### BRIEF DESCRIPTION OF THE DRAWINGS

The invention can be better understood with reference to the following detailed description together with the appended illustrative drawings:

FIGS. 1A-C depict THPC gold-silver alloy seeds on silica nano-particles: (A) UV-vis spectra of pure THPC alloy seeds and deposited alloy seeds on silica nano-particles, (B) TEM image of alloy seeds on silica nano-particles, and (C) FE-SEM image of alloy seeds on silica nano-particles;

FIG. 2 depicts an EDX spectrum of alloy seeds deposited on silica nano-particles;

FIG. 3 depicts UV-vis spectra of alloy seed-gold nano-shell nano-particles;

FIG. 4 depicts TEM images of THPC alloy seed-gold nano-shell nano-particles;

FIGS. 5A-F depict TEM and SEM images of gold, silver, gold-silver alloy nano-shells having a diameter of ˜350 nm silica core nano-particles and a nano-shell thickness of ˜30 nm. TEM images of gold (A), silver (C), and alloy (E) nano-shell and FE-SEM images of gold (B), silver (D), and alloy (F) nano-shells;

FIG. 6 depicts an EDX spectrum showing ˜15 nm gold-silver alloy shells with ˜350 nm silica cores;

FIG. 7 depicts UV-vis spectra of gold, silver, and gold-silver alloy shells having a diameter of ˜350 nm silica cores and a shell thickness of ˜15 nm;

FIG. 8 depicts UV-vis spectra of gold, silver, and gold-silver alloy shells having a diameter of ˜350 nm silica cores and a shell thickness of ˜30 nm;

FIGS. 9A-C depict WV-vis spectra of silver core-gold shell of various sizes and thicknesses of cores and shells: (A) 45 nm silver core with different thicknesses of gold shell, (B) 55 nm silver core thickness with different thicknesses of gold shell, and (C) 75 nm silver core with different thicknesses of gold shell;

FIGS. 10A-D depict TEM images of silver core-gold nano-shell nano-particles;

FIGS. 11A&B depicts FE-SEM images of silver core-gold nano-shell nano-particles;

FIGS. 12A&B depict UV-vis spectra of silica core-silver nano-rod nano-particles;

FIGS. 13 A&B depict FE-SEM images of silica core-silver nano-rod nano-particles: (A) Silver nano rod 71-5 and (B) Silver nano rod 71-6;

FIGS. 14A&B depict TEM images of silica core-silver nano-rod nano-particles: (A) silver nano-rod 73-1 and (B) silver nano-rod 73-6;

FIGS. 15A-D depict FE-SEM images of discrete hydrogel-coated gold particles: (A) discrete hydrogel-coated gold nano-particles (120 nm core), and (B) discrete hydrogel-coated gold nano-particles(100 nm core);

FIG. 16A&B depict TEM images of discrete hydrogel-coated gold particles: (A) discrete hydrogel-coated gold nano-particles (120 nm core) and (B) discrete hydrogel-coated gold nm core);

FIG. 17 illustrates a schematic of a preferred discrete hydrogel coating process;

FIGS. 18A-B depict absorbance spectra of hydrogel-coated gold nano-particles in (A) neutral and (B) acidic or basic media;

FIGS. 19A-D illustrate FE-SEM images of: (A) bare gold nano-particles (˜60 nm), (B) hydrogel-coated gold nano-particles (˜100 nm), (C) hydrogel-coated gold nano-particles (˜130 nm), and (D) hydrogel-coated gold nano-particles (−230 nm);

FIG. 20 depicts an EDX spectrum of hydrogel-coated gold nano-particles;

FIGS. 21A&B depict a plot of particle size verse pH for bare gold nano-particles and hydrogel-coated gold nano-particles and a plot of particle size verses temperature for bare gold nano-particles and hydrogel-coated gold nano-particles, respectively;

FIG. 22 depicts a plot of hydrodynamic diameter (nm) verses temperature during periodic irradiation with light within the plasmon resonance absorption peak;

FIGS. 23A&B depict FE-SEM images of hydrogel-coated gold nano-shells (nano-shell core ˜100 nm with a thin coating) nano-particles;

FIG. 24A&B depict FE-SEM images of hydrogel-coated gold nano-shells (nano-shell core ˜100 nm with a thick coating) nano-particles;

FIGS. 25A&B depict FE-SEM images of hydrogel-coated gold nano-shells (nano-shell core ˜120 nm with a thin coating) nano-particles;

FIGS. 26A&B depict FE-SEM images of 50-60 nm gold nano-particles;

FIGS. 27A&B depict FE-SEM images of 50-60 nm gold nano-particles coated with a gold nano-shell where 7 mL of gold nano-particle solution were used in the preparation;

FIGS. 28A&B depict FE-SEM images of 50-60 nm gold nano-particles coated with a gold nano-shell where 3 mL of gold nano-particle solution were used in the preparation;

FIG. 29 depicts UV-vis spectra of 50-60 nm gold nano-particles with nano-shells prepared with 1 mL, 3 mL, 5 mL, and 7 mL of the 50-60 nm gold nano-particle solution;

FIGS. 30A&B depict FE-SEM images of 50-60 nm gold nano-particles;

FIGS. 31A&B depict FE-SEM images of 50-60 nm gold nano-particles coated with a gold nano-shell where 3 mL of gold nano-particle solution and a low concentration of reducing agent were used in the preparation;

FIGS. 32A-C depict FE-SEM images of 50-60 nm gold nano-particles coated with a gold nano-shell where 5 mL of gold nano-particle solution and a low concentration of reducing agent were used in the preparation;

FIGS. 33A&B depict FE-SEM images of 50-60 nm gold nano-particles coated with a gold nano-shell where 7 mL of gold nano-particle solution and a low concentration of reducing agent were used in the preparation;

FIG. 34 depicts UV-vis spectra of 50-60 nm gold nano-particles with nano-shells prepared with 3 mL, 5 mL, and 7 mL of the 50-60 nm gold nano-particle solution;

FIGS. 35A&B depict FE-SEM images of 10-15 nm gold nano-particles;

FIGS. 36A-C depict FE-FE-SEM images of 10-15 nm gold nano-particles coated with a gold nano-shell where 1 mL of gold nano-particle solution were used in the preparation;

FIGS. 37A&B depict FE-SEM images of 10-15 nm gold nano-particles coated with a gold nano-shell where 3 mL of gold nano-particle solution were used in the preparation;

FIGS. 38A-C depict FE-SEM images of 10-15 nm gold nano-particles coated with a gold nano-shell where 5 mL of gold nano-particles were used in the preparation;

FIGS. 39A&B depict FE-SEM images of 10-15 nm gold nano-particles coated with a gold nano-shell where 9 mL of gold nano-particle solution were used in the preparation;

FIGS. 40A&B depict FE-SEM images of 10-15 nm gold nano-particles coated with a gold nano-shell where 2 mL of gold nano-particle solution were used in the preparation;

FIGS. 41A&B depict FE-SEM images of 10-15 nm gold nano-particles coated with a gold nano-shell where 6 mL of gold nano-particle solution were used in the preparation;

FIGS. 42A&B depict FE-SEM images of 10-15 nm gold nano-particles coated with a gold nano-shell where 11 mL of gold nano-particle solution were used in the preparation;

FIG. 43 depicts UV-vis spectra of 10-15 nm gold nano-particles and nano-shells nano-particles prepared with 1 mL, 2 mL, 3 mL, 5 mL, 6 mL, 7 mL, 9 mL and 11 mL of the 10-15 nm gold nano-particle solution;

FIGS. 44A&B depict FE-SEM images of 50-60 nm silver nano-particles;

FIG. 45 depicts an FE-SEM images of 50-60 nm silver nano-particles coated with a gold nano-shell where 1 mL of silver nano-particle solution were used in the preparation;

FIG. 46 depicts an FE-SEM images of 50-60 nm silver nano-particles coated with a gold nano-shell where 3 mL of silver nano-particle solution were used in the preparation;

FIG. 47 depicts an FE-SEM images of 50-60 nm silver nano-particles coated with a gold nano-shell where 7 mL of silver nano-particle solution were used in the preparation;

FIG. 48 depicts UV-vis spectra of 50-60 nm silver nano-particles and nano-shells nano-particles prepared with 1 mL, 3 mL, 5 mL, and 7 mL of the 50-60 nm silver nano-particle solution;

FIGS. 49A-C depict FE-SEM images of 50-60 nm silver nano-particles coated with a gold nano-shell where 1 mL of silver nano-particle solution and a low concentration of reducing agent were used in the preparation;

FIGS. 50A&B depicts FE-SEM images of 50-60 nm silver nano-particles coated with a gold nano-shell where 3 mL of silver nano-particle solution and a low concentration of reducing agent were used in the preparation;

FIGS. 51A-D depicts FE-SEM images of 50-60 nm silver nano-particles coated with a gold nano-shell where 5 mL of silver nano-particle solution and a low concentration of reducing agent were used in the preparation;

FIGS. 52A&B depicts FE-SEM images of 50-60 nm silver nano-particles coated with a gold nano-shell where 7 mL of silver nano-particle solution were used in the preparation;

FIG. 53 depicts UV-vis spectra of 50-60 nm nano-shells nano-particles prepared with 1 mL, 3 mL, 5 mL, and 7 mL of the 50-60 nm silver nano-particle solution;

FIGS. 54A&B depict FE-SEM images of 10-15 nm silver nano-particles;

FIGS. 55A-C depicts FE-SEM images of 10-15 nm silver nano-particles coated with a gold nano-shell where 1 mL of gold nano-particle solution was used in the preparation;

FIGS. 56A&B depicts FE-SEM images of 10-15 nm silver nano-particles coated with a gold nano-shell where 2 mL of gold nano-particle solution were used in the preparation;

FIGS. 57A-C depicts FE-SEM images of 10-15 nm silver nano-particles coated with a gold nano-shell where 3 mL of gold nano-particle solution were used in the preparation;

FIG. 58 depicts an FE-SEM image of 10-15 nm silver nano-particles coated with a gold nano-shell where 4 mL of the gold nano-particle solution were used in the preparation;

FIG. 59 depicts UV-vis spectra of 10-15 nm nano-shells nano-particles prepared with 1 mL, 2 mL, 3 mL, 4 mL, and 8 mL of the 10-15 nm silver nano-particle solution;

FIG. 60 depicts an FE-SEM image of a 50-60 nm silver nano-particles having gold nano-rods formed thereon to form a sweet gum ball type structure where 1 mL of the silver nano-particle solution;

FIG. 61 depicts an FE-SEM image of a 50-60 nm silver nano-particles having gold nano-rods formed thereon to form a sweet gum ball type structure where 3 mL of the silver nano-particle solution;

FIG. 62 depicts UV-vis spectra of 50-60 nm nano-shells nano-particles prepared with 1 mL, 3 mL, and 5 mL the 50-60 nm silver nano-particle solution;

FIGS. 63A&B depict FE-SEM images of 200 nm hydrogel nano-particles of a homopolymer NIPAM with 50 nm gold nano-particles grown therein;

FIGS. 64A&B depict FE-SEM images of a 500 nm hydrogel nano-particles of a homopolymer NIPAM with 80 nm gold nano-particles grown therein;

FIGS. 65A&B depict FE-SEM images of large ˜100 nm gold nano-particles;

FIGS. 66A&B depict FE-SEM images of a 500 nm hydrogel nano-particles of a co-polymer of acrylic acid and NIPAM having 40 nm gold nano-particles grown therein;

FIGS. 67A&B depict FE-SEM images of a 500 nm hydrogel nano-particles of a co-polymer of acrylic acid and NIPAM having 100 nm gold nano-particles grown therein;

FIG. 68 depicts UV-vis spectra of nano-particles of FIGS. 59-63;

FIGS. 69A&B depict FE-SEM images of 500 nm hydrogel nano-particles of a homopolymer NIPAM with 80 nm gold nano-particles grown therein treated at high temperature before imaging;

FIGS. 70A&B depict FE-SEM images of a 500 nm hydrogel nano-particles of a homopolymer NIPAM with 80 nm gold nano-particles grown therein dried at room temperature with regular imaging;

FIGS. 71A&B depict FE-SEM images of a 500 nm hydrogel nano-particles of a homopolymer NIPAM with 80 nm gold nano-particles grown therein dried at 30° C. under vacuum at 24 hours showing that the hydrogel collapsed to a diameter of 400 nm;

FIGS. 72A&B depict FE-SEM images of a 500 nm hydrogel nano-particles of a homopolymer NIPAM with 80 nm gold nano-particles grown therein dried at 80° C. for 4 hours showing that the hydrogel collapsed to a diameter of 400 nm; and

FIG. 73 depicts UV-vis spectra of nano-particles of FIGS. 65-68.

### DETAILED DESCRIPTION OF THE INVENTION

The inventors have found that nano-shell particles (nano-particles having deposited thereon a metallic nano-shell—partial or complete—or other metallic nano-structure, e.g., nano-rods) can be prepared from oxide, metal oxide, metal, or metal alloy nano-particles or cores having an optionally thin metal or metal alloys shell grown, deposited, or formed thereon to form nano-shell particles with oxide, metal oxide, metal alloy and/or metal cores, where the nano-particles or nano-shell particles have a plasmon resonance so that the nano-particles change temperature upon irradiation with light having a frequency or frequency range at or near (where near means that the light is still converted to heat) the plasmon resonances of the nano-particles and/or nano-shells or have magnetic or electric properties that allow the nano-particles to be detected, inductively heated, or modified due to interactions with applied external fields. These nano-particles and/or nano-shells, many of which are new, novel, and unique, can also include a hydrogel or other polymeric structure formed thereon, deposited thereon, coated thereon, or polymerized thereon to form hydrogel- or polymer-coated nano-particles or nano-shell nano-particles. These hydrogel- or polymer-coated nano-particles possess thermal properties that allow them to undergo a volume change or transition between a collapsed and an expanded state. The transition can be used to deliver a material to: (a) a body site of an animal including a human, (b) a reaction medium, (c) an organic or inorganic matrix, (d) a solution, or (e) any other environment. The transition can also be used to absorb a material instead of delivering a material to a site (body or not), solution, reaction medium, a matrix, or any other environment.

The nano-structures of this invention are ideally suited for heating a site such as a tissue site upon exposure to light having a wavelength in a plasmon resonance of the nano-structures, for releasing an active agent upon heating via exposure to light having a wavelength in a plasmon resonance of the nano-structures, for detonating an explosive upon heating via exposure to light having a wavelength in a plasmon resonance of the nano-structures, for permitting sensing such as via Raman spectroscopy, MR imaging, UV-vis spectroscopy, X-ray imaging, CAT scans, etc.

The present invention broadly relates to new classes of nano-particles. One preferred class of nano-particles includes dielectric nano-particles having thinner and more uniform nano-shells, where the nano-shells comprise a noble metal alloy or are prepared using a noble metal alloy seeding process. Another preferred class includes dielectric nano-particles having metal and/or metal alloy nano-rods formed or grown thereon, where the nano-rods support a plasmon resonance and constitute a discrete, partial, intermittent, nearly continuous, or continuous coating. Another preferred class of nano-particles includes metal and/or metal alloy nano-particles having a nano-shell formed thereon, where the nano-shells are noble metal or noble metal alloy nano-shells. Another preferred class of nano-particles includes metal and/or metal alloy nano-particles having metal and/or metal alloy nano-rods formed or grown thereon, where the nano-rods support a plasmon resonance and constitute a discrete, partial, intermittent, nearly continuous, or continuous coating. Another preferred class of nano-particles includes metal and/or metal alloy nano-particles having a metal or metal alloy nano-shell formed thereon and metal and/or metal alloy nano-rods formed or grown on the nano-shells, where the nano-shell and the nano-rods support a plasmon resonances. Another preferred class of nano-particles includes magnetic (such as ferro-magnetic metal core or magnetically susceptible metal oxide core) nano-particles having a metal or metal alloy nano-shell formed thereon, where the nano-shell supports a plasmon resonances. Another preferred class of nano-particles includes magnetic (such as ferro-magnetic metal core or magnetically susceptible metal oxide core) nano-particles having a metal or metal alloy nano-shell formed thereon and metal and/or metal alloy nano-rods formed or grown on the nano-shells, where the nano-shell and the nano-rods support a plasmon resonance. Another preferred class of nano-particles includes polymer-coated nano-particles as those just described, where the polymer can either thermally dissociate or decompose or undergo a thermally induced change in volume or coating thickness. Another preferred class of nano-particles includes hydrogel coated nano-particles as those just described, where the hydrogel undergoes a thermally induced change in volume or coating thickness. Another preferred class of nano-particles includes nano-particles such as those just described having deposed on their surfaces thermally releasable one or more pharmaceutically active agents. Another preferred class of nano-particles includes polymer-coated or hydrogel-coated nano-particles impregnated with one or more pharmaceutically active agents.

The present invention also broadly relates to methods for producing dielectric nano-particles having thinner and more uniform nano-shells formed thereon. Another preferred method includes producing metal-on-metal nano-shell nano-particles, where the nano-particle and the nano-shell are metallic and where the metals can be the same or different. Another preferred method includes producing metal-on-metal nano-shell nano-particles, where the nano-particle and/or the nano-shell are metal or metal alloys and where the metal or alloy or alloys can be the same or different. Another preferred method includes producing dielectric nano-particles having formed thereon metal or alloy nano-rods. Another preferred method includes producing metal or alloy nano-particles having formed thereon metal or alloy nano-rods. Another preferred method includes producing metal-on-metal or alloy nano-shell nano-particles having metal or alloy nano-rods formed thereon. Another preferred method includes producing polymer-coated nano-particles including the ones described herein or any other nano-particles. Another preferred method includes producing hydrogel-coated nano-particles including the ones described herein or any other nano-particles. Another preferred method includes producing a delivery system by associating agents on the surface of the nano-particles or impregnating the polymer or hydrogel-coated nano-particles with agents, where the agents are released upon heating of the nano-particles either via light, electro-magnetic fields, electric fields, and/or magnetic fields.

The spectral location of the maximum of the plasmon resonance peak for this geometry depends sensitively upon the ratio of the core radius to shell thickness, as well as the dielectric functions of the core and shell. The presence of a dielectric core shifts the plasmon resonance to longer wavelengths relative to a solid nanoparticle made continuously and exclusively of the metallic shell material. For a given core radius, a thin shell will have a plasmon peak that is shifted to longer wavelengths relative to a thicker shell. It is to be emphasized that metal nanoshells possess all of the same technologically viable optical properties as solid metal nanoparticles in addition to this extremely important aspect of resonance tunability. For additional information on metal nano-shell dielectric nano-particle, the reader is directed to U.S. Pat. Nos. 6,344,272; 6,428,811; 6,530,944; 6,645,517; 6,660,381; 6,685,730; 6,685,986; 6,669,724, 6,778,316, and 6,852,252 incorporated herein by reference.

The present invention also relates to a composition including a polymer matrix having dispersed therein a plurality nano-structures of this invention (any of the nano-particles described herein), where the composition has a desired electrical, magnetic, or optical property and where the polymer matrix is any matrix that is relatively inert or has a given property to enhance the electrical, magnetic or optical properties.

The present invention also relates to conducting polymeric wires including a polymeric matrix having nano-particles of this invention (any of the nano-particles described herein) dispersed therein, where the conductivity changes as the polymeric wire is elongated or stretched or compressed. The present invention also relates to a pressures sensor including a polymeric layer including nano-particles dispersed therein, where the layer becomes conductive when compressed with a pressure sufficient bring the conductive nano-particles into a conductively close proximity.

This invention relates in certain regards to a general method for the production of composites including nano-particles of this invention (any of the nano-particles described herein). In particular, the choice of the core material and geometry can be determined independently of the nano-shell, nano-rod, or nano-rod nano-shell material. Similarly, the choice of the nano-shell or nano-rod material and nano-shell thickness or nano-rod dimensions is independent of the desired core nano-particle material. It is also important to note that the nano-shell or nano-rod forming methods and materials described herein will allow for the fabrication of other unique geometries with potentially unique properties; the utility of this method extends far beyond the fabrication of spherical nano-shell nano-particles. For example, coated cubes or pyramids or cylinders, planar surfaces, or structures patterned onto or etched into a planar surface, to name a few, can be easily fabricated using the same methods detailed herein.

The present embodiments have wavelength absorbance maxima in the range of approximately 400 nm to 20 μm. The low wavelength end of the range is defined by the natural plasmon resonance of the metal-like conductor in a nano-shell layer or in nano-rods associated with the surface of the nano-particles. For any given nano-particle, the maximum absorbance depends upon the ratio of the thickness of the core to the thickness of the nano-shell layer or the dimension of the nano-rods or the dimension of a combined nano-shell/nano-rod coating.

The specially tailored nano-particles or nano-particle mixtures of the invention can be added to polymers during their preparation by methods well known in the art. Suitable polymers include polyethylene, polyvinyl alcohol (PVA), latex, nylon, teflon, acrylic, kevlar, epoxy, glasses and the like. Solubility of nano-particles into polymers can be facilitated by functionalization of the nano-particle surfaces with suitable molecules known to those of skill in the art. The resulting coatings and materials can absorb radiation over the wavelength region of the incorporated particles. Embodiments containing these materials can be used in thermal management to produce more energy efficient buildings, automobiles and storage chambers creating savings in air conditioning and heating costs. Fullerene and/or polymer thin film chemistry could be used to incorporate the present materials into photo voltaic devices by methods known in that art. This approach extends the spectral response of solar cells across the infrared region of the solar emission spectrum, providing more efficient solar cells. Similarly, solar cells or similar devices operated in a photoconductive rather than photo voltaic mode could be used to provide new low-cost, compact infrared detectors useful for a range of applications, including but not limited to environmental emissions testing, medical imaging or night vision surveillance.

The compositions of the present invention comprise nano-particles that have at least two layers. At least one layer is immediately adjacent to and surrounds another layer. The innermost layer is referred to be a core or a nano-particle core. In some embodiments, a layer surrounds the core and is referred to as a nano-shell layer so called nano-shell nano-particles. The nano-shell layer is metal-like in that it can conduct electricity and is made of a metal or metal-like material. In one preferred class of nano-particles of this invention, it is preferred that at least one nano-shell layer readily conduct electricity; however, the invention only requires that one nano-shell layer have a lower dielectric constant than the adjacent inner layer or core. In some embodiments, this metal or metal-like nano-shell layer is the outermost layer. In other embodiments, the nano-shell layer immediately adjacent the nano-particle core is not the outer most nano-shell layer. Additional layers, such as a non-conducting layer, a conducting layer, or a sequence of such layers, such as an alternating sequence of non-conducting and conducting layers, maybe bound to this nano-shell layer using the methods described herein and using materials and methods known well to those of skill in the relevant art. Thus, in certain embodiments of this invention the term conductor is defined by reference to the adjacent inner layer (generally the core) and includes any material having a lower dielectric constant than its immediately adjacent inner layer (generally the core). In other embodiments, the conductive layer comprises a plurality of nano-rods and in other embodiments, the conductive layer comprises a nano-shell having a plurality of nano-rods formed thereon.

In certain embodiments, it is preferred that the adjacent inner layer to the nano-shell layer be nonconducting, while in certain other embodiments, the adjacent inner layer is conducting. The so called metal-on-metal nano-shell, nano-rod or nano-rod nano-shell nano-particles appear to violate the premise that the nano-particle core must be a dielectric or insulator and the nano-shell a conductor. The inventor believe that these metal-on-metal nano-shell, nano-rod or nano-rod nano-shell nano-particles are capable of supporting plasmon resonance because the conductivity of the two layers are different. However, the inventors have not ruled out the possibility that a molecular layer of complexing agents such as citric acid or ascorbic acid separates the layer. Regardless of the actual physical/chemical reasons, these metal-on-metal nano-shell, nano-rod or nano-rod nano-shell nano-particles have similar to improved properties over their nano-shell dielectric nano-particles.

For nano-shell dielectric nano-particles, specifically contemplated are nonconducting layers made of dielectric materials and semiconductors. Suitable dielectric materials include, but are not limited to, silicon dioxide, titanium dioxide, polymethyl methacrylate (PMMA), polystyrene, gold sulfide and macromolecules such as dendrimers. In certain embodiments of this invention, the nonconducting layer is comprised of a semiconductor material. For example, core particles may be made of CdSe, CdS or GaAs. The material of the nonconducting layer influences the properties of the particle. For example, if the dielectric constant of the shell layer is larger relative to a particle having a core with a given dielectric constant, the absorbance maximum of the particle will be blue-shifted relative to a particle having a core with a lower dielectric constant. The core may also be a combination or a layered combination of dielectric materials such as those listed above.

One layer of a nano-particle is its core as noted above. In a two layer nano-particle, the core comprises the nonconducting or conducting layer, depending on type of nano-particle. The preferred core is a mono disperse, spherical particle that is easily synthesized in a wide range of sizes, and has a surface that can be chemically derivatized. It is also preferred that nano-particle cores be made of dielectric materials, semiconductors or metals.

Although in preferred embodiments the core is spherical in shape, the nano-particle core may have other shapes such as cubical, cylindrical or hemispherical. Regardless of the geometry of the nano-particle core, it is preferred that the particles be homogenous in size and shape in preferred embodiments. In other embodiments, mixtures are purposefully constructed wherein there is a controlled size and shape distribution. In spherical embodiments, particles have a homogeneous radius that can range from approximately 1 to 10 nanometers to several microns depending upon the desired absorbance maximum of the embodiment. For the purposes of this invention, homogeneity exists when over about 99% of the particles do not vary in diameter by more than 100%. Under this definition a particle preparation wherein 99% of the particles have diameters between about 50 nm to 100 nm would be said to be homogeneous. Specific applications, however, as discussed in the examples, may rely on mixtures of metal nano-shells with different nano-particle core and nano-shell sizes.

Mono disperse colloidal silica is the preferred nonconducting layer or core material for nano-particles having non-conducting cores, while transition metals, noble metals or noble metal alloys are the preferred conducing core material. The dielectric nano-particles can be produced by the base catalyzed reaction of tetraalkoxysilanes, by techniques known well to those of skill in the art. Nearly spherical silica cores having sizes ranging from 10 nm to greater than 4 μm with a variation in particle diameter of only a few percent are preferred. The conductive nano-particles can be prepared by process well known in the art and by those described herein.

In certain embodiments, the nano-shell layer is linked to the dielectric core layer through a linker molecule. Suitable linker molecules include any molecule that is capable of binding both the core and atoms, ions or molecules of the shell. Preferably, linker binding is covalent to both the shell and the inner layer but binding may also be through ionic bonds, lone-pair interactions, hydrogen bonds, Van der Waals interaction or the like. In certain embodiments, the linker binds existing metallic clusters to the surface of a non-conducting layer. In other embodiments, the linker binds atoms, ions or molecules directly to the surface of a non-conducting layer. Thus, in embodiments that have a core made of CdSe, a suitable linker would be able to bind the CdSe core and molecules in the shell. In preferred embodiments, the silicone dioxide core and gold metallic shell, are linked by aminopropyltriethoxy silane (“APTES”).

The present invention also contemplates unique chemical methods for producing the disclosed nano-particles of this invention (any of the nano-particles described herein) in solution. Generally, assembly occurs by way of the following steps. First, core particles are grown or otherwise obtained. Next, a linker molecule is bound to the core. Then, clusters of molecules that comprise the conducting shell layer are reacted with a free reactive end on the linker molecules. These clusters may complete the shell layer or form nucleation sites for the growth of a complete shell layer around the core. For metal core nano-particles, the methods for forming the nano-shell, nano-rods or nano-rod nano-shells are described below.

The conditions under which each of the synthetic reactions is carried out determines the overall size and makeup of the particle. For example, in the synthesis of metal nano-shells, reactants include certain concentrations of metal and reducing equivalents that can be altered along with reaction times to vary the nano-shell thickness and morphology. With certain nano-shell materials, the progress of this reaction can be followed spectrophotometrically due to the distinct absorption peaks of the particles in the visible and infrared regions of the electromagnetic spectrum.

One unique aspect of the present method is the attachment of conducting materials of the nano-shell to the nonconducting inner layer. In the methods of the invention, this step is carried out in solution. In this method, linker molecules that are capable of chemically linking the conducting layer to the core are first bound to the core, e.g., the reaction of APTES with silicon dioxide particles. Other suitable linker molecules include but are not limited to mercaptopropyltrimethoxy silane, 4-aminobutyl dimethoxysilane, and the like. One of skill in the art will readily appreciate that the suitability of a linker molecule depends upon the particular embodiment including the composition of the core and of the conducting shell that will eventually surround the core. With this knowledge, one of skill can identify suitable linkers and bind them to core particles or nonconducting inner layers and then react suitable conducting molecular clusters, ions, or atoms of a suitable conducting material to them.

As one of skill in the art can readily appreciate, suitable solvents for linker molecule attachment depend upon the reactants and a variety of solvents may work under a given set of conditions. The solvent of choice for the attachment of APTES to silicon dioxide is anhydrous ethanol. Generally, where linkers are attached in condensation reactions, the preferred solvents are anhydrous because such solvents tend to drive the reactions to produce more of the desired final reacted product. One of skill in the art would be able to select a suitable solvent based on chemical methodologies well known in the chemical arts.

Once the linker molecules are bound to the dielectric nano-particle core, a free reactive moiety on the linker is reacted with clusters of molecules, ions or atoms to produce all or part of a conducting nano-shell. In certain embodiments, the clusters are metal atoms. Metal clusters, ions or atoms that are linked to the core particle through a linker molecule are said to be “tethered.” In certain embodiments the tethered metal atoms or clusters serve as nucleation sites for the deposition of additional metal from solution. In other embodiments, the attachment of metal clusters completes the synthesis. A similar methodology is used when forming metal nano-shells or nano-rods on metal nano-particle cores or when forming nano-rods on dielectric nano-particle cores.

Generally, metal is deposited onto the tethered clusters and enlarges the clusters until a coherent metal nano-shell of the desired thickness is formed. The metal can be deposited through reduction process of solution metal onto the tethered clusters. Alternatively, metal can be deposited on the tethered metal clusters by a “colloid-based” deposition process. The deposition can also be initiated or driven photochemically. The technique of depositing metal onto metal nucleation sites tethered to nonconducting core materials in solution is one of the novel features of the present methods.

In certain preferred embodiments, the metallic nano-shell is the terminal layer. However, attachment of molecules or additional layers can change the physical properties of the particle. A chemical or charge-transfer interaction between the metallic nano-shell and an additional layer, or just the local embedding medium, influences the optical absorption of the particles, as discussed by Kreibig et al, incorporated herein by reference to the extent it provides such methods.

In addition, the near field of the metallic nano-shell can affect the properties of molecules adsorbed on the surface of the nano-particles. This could be of use in chemical sensing applications. In other embodiments, a non-conducting layer surrounding the metallic layer can provide a steric barrier that is useful when processing or organizing the nano-particles into a particular arrangement. Chemical functionalization of the metal surface is also useful for transferring the metal nano-shells between different solvents, as discussed by Sarathy et al., incorporated herein by reference to the extent it provides such methods. Chemical functionalization may also assist or enable the formation of arrays or crystals of these nano-particles, which will possess additional unique optical properties relating to the periodicity of the array or crystal structure, in similarity with photonic band gap crystals and arrays.

By varying the conditions of the metal deposition reaction, the ratio of the thickness of the metal nano-shell to the nonconducting or conducting inner layer can be varied in a predictable and controlled way. Nano-particles can be constructed with metallic nano-shell layer to nano-particle core layer radius with ratios from 10 to 10⁻³. This large ratio range coupled with control over the nano-particle core size results in a particle that has a large, frequency-agile absorbance over most of the visible and infrared regions of the spectrum.

There are many possible applications of nano-particles of this invention that could utilize the tunability of the plasmon resonance. The nano-particles of this invention could be made to absorb or scatter light at specific wavelengths in the visible or infrared range. Such compositions would be ideal for use in a wide range of materials including energy efficient paints, windows, coatings, or fabrics that could be used on or in vehicles and building structures. The nano-particle compositions of this invention could be suspended as an active agent in inks, for cryptographic marking purposes. These materials would also be particularly well suited for use in air heating units or in solar collector materials. Such a solar absorber could also be used as a shield or screen that absorbs or scatters incident solar radiation, keeping the structure cooler than if it were directly exposed to the solar radiation.

Such materials could be useful in many other applications to efficiently “manage” the radiation from any thermal source. For example, these compositions could be adsorbed onto or embedded into materials, thin films, coatings, or fabrics that convert radiation directly into heat (passive solar energy harvesting), or into devices or device components, that convert radiation into electricity via photo voltaic or photoconductive effects, or that convert radiation into chemical energy (fuel cells). Mixtures of these compositions could be made to absorb or scatter solar energy across the entire solar radiation spectrum.

These nano-particles of this invention (any of the nano-particles described herein) could be used to sensitize existing photo voltaic, photoconductive, or bolometric devices for enhanced photoresponse and efficiency, and could be used as the functional basis for new device designs. The strong infrared photoresponse of these compositions may be useful for sensitization of many different types of semiconductor or polymer surfaces or films for other applications.

For example, the selective infrared absorption may be useful for laser eye protection, or eye protection from other potentially damaging sources of infrared radiation. The enhanced optical field in the vicinity (1-20 nm) of a nano-particle or mixtures or combinations of nano-particles of this invention may facilitate photochemistry or photoelectrochemistry, either on the nano-particle surface, on a substrate upon which the nano-particle is attached, or an electrode upon which the nano-particle is attached or embedded. Structures containing such compositions could be used in photoconductive applications such as in infrared detectors. Infrared detectors utilizing the properties of these compositions could be used in a wide range of applications such as detecting emissions in environmental monitoring, optical telecommunications networks, wavelength selective, mid-infrared detectors for medical imaging, night vision surveillance equipment or infrared telescopes.

Compositions of this invention constructed with different resonant frequencies could be selectively manipulated, levitated, or “sifted” using the wavelength dependent dipole force of a laser beam or beams. Additionally, nano-shell, nano-rod, nano-rod nano-shell nano-particles or mixtures or combinations thereof of this invention, where the nano-particle cores are conducting, semi-conducting or non-conducting, can be made that possess unique electronic properties that could be useful in specific electronic device applications. The fabrication of homogeneous nano-shell, nano-rod, nano-rod nano-shell comprised of several hundred or a few thousand atoms covering conducting, semi-conducting or non-conducting nano-particle cores as small as 1 nm would have well-defined electronic energy levels, similar to molecules, whose energy level spacings are controllably defined by the nano-shell geometry as described by Puska and Neiminen, incorporated herein by reference to the extent it provides such methods.

In other words, the energy eigenstates of very small diameter metal nano-shell, nano-rod, nano-rod nano-shell nano-particles or mixtures or combinations thereof of this invention, where the nano-particle cores are conducting, semi-conducting or non-conducting, are defined not only by the shell thickness, but by the diameter of the inner core as well. For small core diameters, both the optical and electronic properties are unique to the ultra small core/shell structure. Such nano-particles of this invention might find application in nanoscale devices, such as single electron transistors or coulomb blockade devices that rely on having well defined electronic energy level spacings. They may also provide useful electronic or electrical properties as components of larger devices. In addition, there could be higher energy optical resonances of metal nano-shells, nano-rods or nano-shell/nano-rods that lie in the vacuum ultraviolet or X-ray region of the electromagnetic spectrum, a property that could be applied to the fabrication of X-ray absorbers or detectors.

The enhanced polarizability at the plasmon resonance of these compositions could be used in chemical sensing or chemical analysis applications, where information concerning the properties of molecules adsorbed onto the nano-particle surface is obtained. Such compositions may permit the use of surface enhanced raman scattering (SERS) to be performed upon adsorbate or adjacent molecules using laser wavelengths in the near-infrared or infrared region of the spectrum. For compositions prepared where the shell is incomplete, second-order nonlinear optical effects may be enhanced when such oriented compositions are adsorbed onto a surface or embedded into an appropriate medium.

In accordance with the present invention, a composition for modulated in vivo drug-delivery to a subject (animal or human) in need thereof is provided. In certain embodiments the composition comprises a plurality of heat generating nano-particles of this invention (one type or a mixture of types). Each of these particles has a conducting, semi-conducting or non-conducting nano-particle core with an independently defined radius, a metal nano-shell, metal nano-rods or metal nano-shell with nano-rods adhering to the nano-particle core and also having an independently defined thickness (nano-shell) or dimension (nano-rods). The terms “independently defined radius” and “independently defined thickness or dimension” mean that the desired thickness or dimension of each of the nano-shell, nano-rod and nano-particle core can be chosen and formed without dictating the thickness of the other. Each particle also includes a defined core radius:shell thickness ratio or core radius:rod dimension ratio, and a defined wavelength absorbance maximum in a region of the electromagnetic spectrum preferably in the near-infrared range of the electromagnetic spectrum. In preferred embodiments, the nano-shell, nano-rod, or nano-rod nano-shell and nano-particle core are joined by a linker molecule. The composition may be in the form of a dry composite hydrogel, suitable for being rehydrated at a later time and loaded with a drug in aqueous solution. In certain embodiments the composite contains at least one therapeutic or pharmaceutically active agent, such as a drug or a biologically active material, and a suitable medium, support or carrier in a hydrated form. The medium comprises a thermally responsive material in contact with the particles. The necessary thermal contact may be establishment of a polymer/particle interface, by chemical binding of the particle surface to the polymer, or the like. The therapeutic or pharmaceutically active agent is reversibly contained in the composition when the temperature of the composition is at or below approximately normal body temperature of a subject, e.g., about 37° C. In some embodiments, the agent is reversibly released from the composition when the temperature is about 40° C. or more. In preferred embodiments, the medium contains a polymer hydrogel in which the thermally responsive material is substantially solid at normal body temperature of the subject (e.g., 37° C.) and undergoes a reversible phase transition at temperatures about 3 or more ° C. above normal (e.g., 40° C.), and preferably between about 40-45° C. The thermally responsive material may comprise more than one polymer in some embodiments. The particles of the composition are of such design that they convert incident radiation into heat energy when they are irradiated by light of a defined wavelength.

In other preferred embodiments, the nano-particles of this invention (any nano-particles described herein) are either individually coated with a bio-compatible polymer such as a hydrogel or are formed within a pre-made bio-compatible polymer or hydrogel nano-particle. A pharmaceutically active agent can then be impregnated into the bio-polymer or hydrogel for delivery of the coated nano-particles into an animal or human body.

Certain preferred embodiments of the nano-particles of the invention comprise a gold sulfide core and a gold shell. In certain other embodiments the core comprises silicon dioxide and the shell comprises gold. In certain embodiments, optically tuned nano-shells are embedded within a polymer matrix. In certain embodiments, nano-shells are embedded in the surface of a N-isopropylacrylamide and acrylamide hydrogel. In certain other embodiments, the nano-shells and polymer together form microparticles, nano-particles, or vesicles. In some embodiments the particle core is between about 1 nm up to slightly less than 5 μm in diameter, the shell is about 1-100 nm thick, and the particle has an absorbance maximum wavelength of about 300 nm to 20 μm, preferably in the near-infrared range. In other embodiments, the nano-particles of this invention comprise gold or silver nano-particle cores and a gold or gold alloy nano-shell, gold or gold alloy nano-rods, or gold or gold alloy nano-rods on gold or gold alloy nano-shell.

Another aspect of the present invention provides optically heatable nano-particles suitable for use in the new compositions described above. The nano-particles effectively convert incident electromagnetic radiation into heat energy when they are irradiated. The conversion of incident electromagnetic radiation into heat energy is optimized when the incident radiation is at the defined wavelength at which the nano-particles' absorbance is at its maximum.

Still another aspect of the invention provides a system for modulated in vivo delivery of a therapeutic agent. According to certain embodiments, the system comprises an implantable composition containing a plurality of photo thermally responsive nano-particles, as described herein, at least one therapeutic agent, and a medium. The medium comprises a thermally responsive material in contact with the nano-particles and is characterized as described herein. The modulated in vivo delivery system may optionally include a biosensor system, for providing information about in vivo status to assist in making treatment decisions. If desired, the composition may be contained in an implantable porous or permeable device.

In still another aspect of the invention, a method of photo thermally modulating in vivo delivery of a therapeutic agent is provided. According to certain embodiments, the method includes implanting into the body of a subject in need of treatment, a composition or a device containing a plurality of nano-particles of this invention or mixture or combination thereof, at least one therapeutic agent, and a medium. The composition, which may be any suitable composition described herein, includes a thermally responsive material in contact with the nano-particles of this invention or mixtures or combinations thereof. Preferably the material has a defined lower critical solution temperature that is slightly above the normal body temperature of the subject. The therapeutic or pharmaceutically active agent is substantially retained by the composition when the temperature of the composition is at about normal body temperature of the subject. At least a portion of the agent is substantially released from the composition into the body of the subject when the temperature of the composition, or a portion thereof, is raised to the lower critical solution temperature. The method includes applying electromagnetic radiation, preferably near-infrared, to the implanted composition or device from outside the body (animal including human). The amount and duration of irradiation is sufficient to raise the temperature of the nano-particles such that the composition, or a portion thereof, is raised to the lower critical solution temperature, causing release of the agent to commence. Application of the radiation in continued until a desired amount of the agent has been released from the composition into the body. After all or the desired of the agent has been delivered, the composition is allowed to return to normal body temperature, whereupon drug delivery is reduced or ceased, as desired. In some embodiments of the method, the irradiation is repeated at a later time, if multiple dosing is desired.

In an important embodiment of the present invention, the nano-particles of this invention or mixtures or combinations thereof administered to an animal including a human using standard methods. Animals that may be treated using the method of the invention include, but are not limited to humans, cows, horses, pigs, dogs, cats, sheep goats, rabbits, rats, mice, birds, chickens or fish.

A method to selectively image or kill cells and/or tissue for diagnostic and therapeutic applications has been developed. The particles are ideally of nanometer-scale dimensions. The method may include targeting schemes involving specific chemical interactions (e.g., antigen-antibody binding, etc.) or may consist of the simple delivery of the therapeutic reagents to the desired area. The direction or targeting of the therapy may be to the surface of the subject cells and/or tissue, or it may be to other, interior sites. Several new classes of such nano-particles that offer more specific and accurate imaging technologies, based on nano-particles of this invention or mixtures or combinations thereof that emit or scatter near infrared light and that can be easily conjugated to antibodies, as well as highly localized, targeted, and minimally invasive treatment strategies based on photo thermal interactions with nano-particles, have been developed. In a preferred embodiment to kill the targeted cells, the nano-particles are nano-shells and are formed with a core of a dielectric or inert material such as silicon, a semi-conductive material or a conductive materials (a noble metal or an alloy thereof), coated with a material such as a highly conductive metal and/or having rods of a highly conductive metal formed thereon which can be excited using radiation such as near infrared light (approximately 800 to 1300 nm). Upon excitation, the nano-particles of this invention or mixtures or combinations thereof emit heat. The combined dimension of the nano-shell and/or nano-rods nano-particles of this invention ranges from the tens to the hundreds of nanometers.

Importantly, in all embodiments of the present invention, the excitation maybe effected from an excitation source inside the material to which hyperthermia is to be induced or it may be effected by an excitation source outside the material. In the in vivo applications, it may be effected by an excitation source inside the body or outside the body. In in vivo applications wherein the excitation source is inside the body, the excitation source may be in the subject material or outside it.

Near infrared light is advantageous for its ability to penetrate tissue. Other types of radiation can also be used, depending on the selection of the coated nano-particles of this invention and targeted cells. Examples include X-rays, electromagnetic fields, magnetic fields, electric fields, and ultrasonic fields. The problems with the existing methods for hyperthermia, especially for use in cancer therapy, such as the use of heated probes, microwaves, ultrasound, lasers, perfusion, radiofrequency energy, and radiant heating is avoided since the levels of radiation used as described herein is insufficient to induce hyperthermia except at the surface of the nano-particles, where the energy is more effectively concentrated by the metal structures on the surface of the nano-particle core, whether the core material is conducting, semi-conducting, non-conducting, ferromagnetic, or having a high magnetic susceptibility such as an iron oxide core. The nano-particles of this invention or mixtures or combinations thereof can also be used to enhance imaging, especially using infrared diffuse photon imaging methods. If targeting molecules are used in conjunction with the coated nano-particles of this invention, the targeting molecules can be antibodies or fragments thereof, ligands for specific receptors, or other proteins specifically binding to the surface of the cells to be targeted. However, the administration of coated nano-particles of this invention including a pharmaceutically active agent does not require targeting molecules as the nano-particles appear to concentrate in regions of high proliferation such as cancers.

Materials and methods are described to deliver nano-particles that scatter, absorb, and/or emit near infrared light to cells; to use these as contrast agents or emitters to optically tag cells for near-IR imaging; to provide infrared tomographic imaging methods based on these specifically tagged cells and to photo thermally target the destruction of individual cells by optically exciting the nano-particle tags with near infrared light.

Aqueous compositions of the present invention comprise an effective amount of the nano-particles of this invention or mixtures or combinations thereof dissolved and/or dispersed in a pharmaceutically acceptable carrier and/or aqueous medium.

The phrases pharmaceutically and/or pharmacologically acceptable refer to molecular entities and/or compositions that do not produce an adverse, allergic and/or other untoward reaction when administered to an animal, as appropriate.

As used herein, pharmaceutically acceptable carrier includes any and/or all solvents, dispersion media, coatings, antibacterial and/or antifungal agents, isotonic and/or absorption delaying agents and/or the like. The use of such media and/or agents for pharmaceutical active substances is well known in the art. Except insofar as any conventional media and/or agent is incompatible with the active ingredient, its use in the therapeutic compositions is contemplated. Supplementary active ingredients can also be incorporated into the compositions. For administration, preparations should meet sterility, pyrogenicity, general safety and/or purity standards as required by FDA Office of Biologics standards.

The biological material should be extensively dialyzed to remove undesired small molecular weight molecules and/or lyophilized for more ready formulation into a desired vehicle, where appropriate. The active compounds may generally be formulated for parenteral administration, e.g., formulated for injection via the intravenous, intramuscular, sub-cutaneous, intralesional, and/or even intraperitoneal routes. The preparation of an aqueous compositions that contain an effective amount of the nano-particles of this invention or mixtures or combinations thereof as an active component and/or ingredient will be known to those of skill in the art in light of the present disclosure. Typically, such compositions can be prepared as injectables, either as liquid solutions and/or suspensions; solid forms suitable for using to prepare solutions and/or suspensions upon the addition of a liquid prior to injection can also be prepared; and/or the preparations can also be emulsified.

The pharmaceutical forms suitable for injectable use include sterile aqueous solutions and/or dispersions; formulations including sesame oil, peanut oil and/or aqueous propylene glycol; and/or sterile powders for the extemporaneous preparation of sterile injectable solutions and/or dispersions. In all cases the form must be sterile and/or must be fluid to the extent that easy syringability exists. It must be stable under the conditions of manufacture and/or storage and/or must be preserved against the contaminating action of microorganisms, such as bacteria and/or fungi.

Solutions of the active compounds as free base and/or pharmacologically acceptable salts can be prepared in water suitably mixed with a surfactant, such as hydroxypropylcellulose. Dispersions can also be prepared in glycerol, liquid polyethylene glycols, and/or mixtures thereof and/or in oils. Under ordinary conditions of storage and/or use, these preparations contain a preservative to prevent the growth of microorganisms.

The nano-particles of the present invention or mixtures or combinations thereof can be formulated into a composition in a neutral and/or salt form. Pharmaceutically acceptable salts, include the acid addition salts (formed with the free amino groups of the protein) and/or which are formed with inorganic acids such as, for example, hydrochloric and/or phosphoric acids, and/or such organic acids as acetic, oxalic, tartaric, mandelic, and/or the like. Salts formed with the free carboxyl groups can also be derived from inorganic bases such as, for example, sodium, potassium, ammonium, calcium, and/or ferric hydroxides, and/or such organic bases as isopropylamine, trimethylamine, histidine, procaine and/or the like.

The carrier can also be a solvent and/or dispersion medium containing, for example, water, ethanol, polyol (for example, glycerol, propylene glycol, and/or liquid polyethylene glycol, and/or the like), suitable mixtures thereof, and/or vegetable oils. The proper fluidity can be maintained, for example, by the use of a coating, such as lecithin, by the maintenance of the required particle size in the case of dispersion and/or by the use of surfactants. The prevention of the action of microorganisms can be brought about by various antibacterial and/or antifungal agents, for example, parabens, chlorobutanol, phenol, sorbic acid, thimerosal, and/or the like. In many cases, it will be preferable to include isotonic agents, for example, sugars and/or sodium chloride. Prolonged absorption of the injectable compositions can be brought about by the use in the compositions of agents delaying absorption, for example, aluminum monostearate and/or gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the required other ingredients from those enumerated above. In the case of sterile powders for the preparation of sterile injectable solutions, the preferred methods of preparation are vacuum-drying and/or freeze-drying techniques which yield a powder of the active ingredient plus any additional desired ingredient from a previously sterile-filtered solution thereof The preparation of more, and/or highly, concentrated solutions for direct injection is also contemplated, where the use of DMSO as solvent is envisioned to result in extremely rapid penetration, delivering high concentrations of the active agents to a small tumor area.

Upon formulation, solutions will be administered in a manner compatible with the dosage formulation and/or in such amount as is therapeutically effective. The formulations are easily administered in a variety of dosage forms, such as the type of injectable solutions described above, but drug release capsules and/or the like can also be employed.

For parenteral administration in an aqueous solution, for example, the solution should be suitably buffered if necessary and/or the liquid diluent first rendered isotonic with sufficient saline and/or glucose. These particular aqueous solutions are especially suitable for intravenous, intramuscular, subcutaneous and/or intraperitoneal administration. In this connection, sterile aqueous media which can be employed will be known to those of skill in the art in light of the present disclosure. For example, one dosage could be dissolved in 1 mL of isotonic NaCl solution and/or either added to 1000 mL of hypodermoclysis fluid and/or injected at the proposed site of infusion, (see for example, “Remington's Pharmaceutical Sciences” 15th Edition, pages 1035-1038 and/or 1570-1580). Some variation in dosage will necessarily occur depending on the condition of the subject being treated. The person responsible for administration will, in any event, determine the appropriate dose for the individual subject.

In addition to the compounds formulated for parenteral administration, such as intravenous and/or intramuscular injection, other pharmaceutically acceptable forms include, e.g., tablets and/or other solids for oral administration; liposomal formulations; time release capsules; and/or any other form currently used, including cremes.

One may also use nasal solutions and/or sprays, aerosols and/or inhalants in the present invention. Nasal solutions are usually aqueous solutions designed to be administered to the nasal passages in drops and/or sprays. Nasal solutions are prepared so that they are similar in many respects to nasal secretions, so that normal ciliary action is maintained. Thus, the aqueous nasal solutions usually are isotonic and/or slightly buffered to maintain a pH of 5.5 to 6.5. In addition, antimicrobial preservatives, similar to those used in ophthalmic preparations, and/or appropriate drug stabilizers, if required, may be included in the formulation.

Additional formulations which are suitable for other modes of administration include vaginal suppositories and/or pessaries. A rectal pessary and/or suppository may also be used. Suppositories are solid dosage forms of various weights and/or shapes, usually medicated, for insertion into the rectum, vagina and/or the urethra. After insertion, suppositories soften, melt and/or dissolve in the cavity fluids. In general, for suppositories, traditional binders and/or carriers may include, for example, polyalkylene glycols and/or triglycerides; such suppositories may be formed from mixtures containing the active ingredient in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate and/or the like. These compositions take the form of solutions, suspensions, tablets, pills, capsules, sustained release formulations and/or powders. In certain defined embodiments, oral pharmaceutical compositions will comprise an inert diluent and/or assimilable edible carrier, and/or they may be enclosed in hard and/or soft shell gelatin capsule, and/or they may be compressed into tablets, and/or they may be incorporated directly with the food of the diet. For oral therapeutic administration, the active compounds may be incorporated with excipients and/or used in the form of ingestible tablets, buccal tables, troches, capsules, elixirs, suspensions, syrups, wafers, and/or the like. Such compositions and/or preparations should contain at least 0.1% of active compound. The percentage of the compositions and/or preparations may, of course, be varied and/or may conveniently be between about 2 to about 75% of the weight of the unit, and/or preferably between 25-60%. The amount of active compounds in such therapeutically useful compositions is such that a suitable dosage will be obtained.

The tablets, troches, pills, capsules and/or the like may also contain the following: a binder, as gum tragacanth, acacia, cornstarch, and/or gelatin; excipients, such as dicalcium phosphate; a disintegrating agent, such as corn starch, potato starch, alginic acid and/or the like; a lubricant, such as magnesium stearate; and/or a sweetening agent, such as sucrose, lactose and/or saccharin may be added and/or a flavoring agent, such as peppermint, oil of wintergreen, and/or cherry flavoring. When the dosage unit form is a capsule, it may contain, in addition to materials of the above type, a liquid carrier. Various other materials may be present as coatings and/or to otherwise modify the physical form of the dosage unit. For instance, tablets, pills, and/or capsules may be coated with shellac, sugar and/or both. A syrup of elixir may contain the active compounds sucrose as a sweetening agent methyl and/or propylparabens as preservatives, a dye and/or flavoring, such as cherry and/or orange flavor.

The examples of pharmaceutical preparations described above are merely illustrative and not exhaustive; the nano-particles of the present invention or mixtures or combinations thereof are amenable to most common pharmaceutical preparations.

### Lipids and Liposome Delivery Methods

Other delivery methods of the present invention comprise a novel composition comprising one or more lipids associated with at least one nano-particles of the present invention or mixtures or combinations thereof. A lipid is a substance that is characteristically insoluble in water and extractable with an organic solvent. Lipids include, for example, the substances comprising the fatty droplets that naturally occur in the cytoplasm as well as the class of compounds which are well known to those of skill in the art which contain long-chain aliphatic hydrocarbons and their derivatives, such as fatty acids, alcohols, amines, amino alcohols, and aldehydes. Of course, compounds other than those specifically described herein that are understood by one of skill in the art as lipids are also encompassed by the compositions and methods of the present invention. This invention also encompasses other host-guest complexation schemes such as those wherein the host molecules may be crown ethers, cyclodextrins, micelles, among others.

A lipid maybe naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof

In particular embodiments, a lipid comprises a liposome. A liposome is a generic term encompassing a variety of single and multilamellar lipid vehicles formed by the generation of enclosed lipid bilayers or aggregates. Liposomes may be characterized as having vesicular structures with a bilayer membrane, generally comprising a phospholipid, and an inner medium that generally comprises an aqueous composition.

A multilamellar liposome has multiple lipid layers separated by aqueous medium. They form spontaneously when lipids comprising phospholipids are suspended in an excess of aqueous solution. The lipid components undergo self-rearrangement before the formation of closed structures and entrap water and dissolved solutes between the lipid bilayers (Ghosh and Bachhawat, 1991). Lipophilic molecules or molecules with lipophilic regions may also dissolve in or associate with the lipid bilayer.

In particular embodiments, a lipid and/or nano-particles of the present invention or mixtures or combinations thereof may be, for example, encapsulated in the aqueous interior of a liposome, interspersed within the lipid bilayer of a liposome, attached to a liposome via a linking molecule that is associated with both the liposome and the nano-particles of the present invention or mixtures or combinations thereof, entrapped in a liposome, complexed with a liposome, etc.

A liposome used according to the present invention can be made by different methods, as would be known to one of ordinary skill in the art. Phospholipids can form a variety of structures other than liposomes when dispersed in water, depending on the molar ratio of lipid to water. At low ratios the liposome is the preferred structure. Liposomes can be prepared in accordance with other known laboratory procedures (e.g., see Bangham et al., 1965; Gregoriadis, 1979; Deamer and Uster 1983, Szoka and Papahadjopoulos, 1978, each incorporated herein by reference in relevant part). These methods differ in their respective abilities to entrap aqueous material and their respective aqueous space-to-lipid ratios.

The size of a liposome varies depending on the method of synthesis. Liposomes in the present invention can be a variety of sizes. In certain embodiments, the liposomes are small, e.g., less than about 100 nm, about 90 nm, about 80 nm, about 70 nm, about 60 nm, or less than about 50 nm in external diameter. In preparing such liposomes, any protocol described herein, or as would be known to one of ordinary skill in the art may be used. Additional non-limiting examples of preparing liposomes are described in U.S. Pat. Nos. 4,728,578, 4,728,575, 4,737,323, 4,533,254, 4,162,282, 4,310,505, and 4,921,706; International Applications PCT/US85/01161 and PCT/US89/05040; U.K. Patent Application GB 2193095 A; Mayer et al., 1986; Hope et al., 1985; Mayhew et al. 1987; Mayhew et al., 1984; Cheng et al., 1987; and Liposome Technology, 1984, each incorporated herein by reference.

Liposomes interact with cells to deliver agents via four different mechanisms: Endocytosis by phagocytic cells of the reticuloendothelial system such as macrophages and/or neutrophils; adsorption to the cell surface, either by nonspecific weak hydrophobic and/or electrostatic forces, and/or by specific interactions with cell-surface components; fusion with the plasma cell membrane by insertion of the lipid bilayer of the liposome into the plasma membrane, with simultaneous release of liposomal contents into the cytoplasm; and/or by transfer of liposomal lipids to cellular and/or subcellular membranes, and/or vice versa, without any association of the liposome contents. Varying the liposome formulation can alter which mechanism is operative, although more than one may operate at the same time.

Targeted delivery is achieved by the addition of ligands without compromising the ability of these liposomes deliver large amounts of nano-shells. It is contemplated that this will enable delivery to specific cells, tissues and organs. The targeting specificity of the ligand-based delivery systems are based on the distribution of the ligand receptors on different cell types. The targeting ligand may either be non-covalently or covalently associated with the lipid complex, and can be conjugated to the liposomes by a variety of methods.

The targeting ligand can be either anchored in the hydrophobic portion of the complex or attached to reactive terminal groups of the hydrophilic portion of the complex. The targeting ligand can be attached to the liposome via a linkage to a reactive group, e.g., on the distal end of the hydrophilic polymer. Preferred reactive groups include amino groups, carboxylic groups, hydrazide groups, and thiol groups. The coupling of the targeting ligand to the hydrophilic polymer can be performed by standard methods of organic chemistry that are known to those skilled in the art. In certain embodiments, the total concentration of the targeting ligand can be from about 0.01 to about 10% mol.

Targeting ligands are any ligand specific for a characteristic component of the targeted region. Preferred targeting ligands include proteins such as polyclonal or monoclonal antibodies, antibody fragments, or chimeric antibodies, enzymes, or hormones, or sugars such as mono-, oligo- and poly-saccharides (see, Heath et al 1986) For example, disialoganglioside GD2 is a tumor antigen that has been identified neuroectodermal origin tumors, such as neuroblastoma, melanoma, small-cell lung carcenoma, glioma and certain sarcomas (Mujoo et al., 1986, Schulz et al., 1984). Liposomes containing anti-disialoganglioside GD2 monoclonal antibodies have been used to aid the targeting of the liposomes to cells expressing the tumor antigen (Montaldo et al., 1999; Pagan et al., 1999). In another non-limiting example, breast and gynecological cancer antigen specific antibodies are described in U.S. Pat. No. 5,939,277, incorporated herein by reference. In a further non-limiting example, prostate cancer specific antibodies are disclosed in U.S. Pat. No. 6,107,090, incorporated herein by reference. Thus, it is contemplated that the antibodies described herein or as would be known to one of ordinary skill in the art may be used to target specific tissues and cell types in combination with the compositions and methods of the present invention. In certain embodiments of the invention, contemplated targeting ligands interact with integrins, proteoglycans, glycoproteins, receptors or transporters. Suitable ligands include any that are specific for cells of the target organ, or for structures of the target organ exposed to the circulation as a result of local pathology, such as tumors.

In certain embodiments of the present invention, in order to enhance the transduction of cells, to increase transduction of target cells, or to limit transduction of undesired cells, antibody or cyclic peptide targeting moieties (ligands) are associated with the lipid complex. Such methods are known in the art. For example, liposomes have been described further that specifically target cells of the mammalian central nervous system (U.S. Pat. No. 5,786,214, incorporated herein by reference). The liposomes are composed essentially of N-glutarylphosphatidylethanolamine, cholesterol and oleic acid, wherein a monoclonal antibody specific for neuroglia is conjugated to the liposomes. It is contemplated that a monoclonal antibody or antibody fragment may be used to target delivery to specific cells, tissues, or organs in the animal, such as for example, brain, heart, lung, liver, etc.

Still further, a nano-shell may be delivered to a target cell via receptor-mediated delivery and/or targeting vehicles comprising a lipid or liposome. These take advantage of the selective uptake of macromolecules by receptor-mediated endocytosis that will be occurring in a target cell. In view of the cell type-specific distribution of various receptors, this delivery method adds another degree of specificity to the present invention.

Thus, in certain aspects of the present invention, a ligand will be chosen to correspond to a receptor specifically expressed on the target cell population. A cell-specific nano-shell delivery and/or targeting vehicle may comprise a specific binding ligand in combination with a liposome. The nano-shell to be delivered are housed within a liposome and the specific binding ligand is functionally incorporated into a liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a nucleic acid to cells that exhibit upregulation of the EGF receptor.

In still further embodiments, the specific binding ligand may comprise one or more lipids or glycoproteins that direct cell-specific binding. For example, lactosyl-ceramide, a galactose-terminal asialganglioside, have been incorporated into liposomes and observed an increase in the uptake of the insulin gene by hepatocytes (Nicolau et al., 1987). The asialoglycoprotein, asialofetuin, which contains terminal galactosyl residues, also has been demonstrated to target liposomes to the liver (Spanjer and Scherphof, 1983; Hara et al., 1996). The sugars mannosyl, fucosyl or N-acetyl glucosamine, when coupled to the backbone of a polypeptide, bind the high affinity manose receptor (U.S. Pat. No. 5,432,260, specifically incorporated herein by reference in its entirety). It is contemplated that the cell or tissue-specific transforming constructs of the present invention can be specifically delivered into a target cell or tissue in a similar manner.

In another example, lactosyl ceramide, and peptides that target the LDL receptor related proteins, such as apolipoprotein E3 (“Apo E”) have been useful in targeting liposomes to the liver (Spanjer and Scherphof, 1983; WO 98/0748, incorporated herein by reference).

Folate and the folate receptor have also been described as useful for cellular targeting (U.S. Pat. No. 5,871,727). In this example, the vitamin folate is coupled to the complex. The folate receptor has high affinity for its ligand and is overexpressed on the surface of several malignant cell lines, including lung, breast and brain tumors. Anti-folate such as methotrexate may also be used as targeting ligands. Transferrin mediated delivery systems target a wide range of replicating cells that express the transferrin receptor (Gilliland et al., 1980).

### Binding of Conjugated Nano-particles to Cultured Cells

Nano-particles of the present invention or mixtures or combinations thereof (absorber/scatterers and emitters) can be linked to cell-specific antibodies or peptides in order to cause targeted binding of an injectable nano-particle formulation to a specific tissue or cell type, particularly cancerous prostate epithelial cells. nano-particles of the present invention or mixtures or combinations thereof and nanoemitters can be prepared with surface-bound, cell-specific antibodies, such as antibodies directed against prostate specific membrane antigen. Cultured cells that are either targeted for nano-particles of the present invention or mixtures or combinations thereof conjugate binding or that serve as non-specific controls are exposed to nano-particle suspensions then rinsed thoroughly to remove unbound nano-particles. Nano-particle binding to cell surfaces can be assessed via environmental scanning electron microscopy (ESEM).

### In Vitro and In Vivo Procedures

A skilled artisan realizes that the nano-particles of the present invention or mixtures or combinations thereof can be employed in a variety of types of experimental procedures, for example, but not limited to in vitro or in vivo experimental procedures.

Briefly, in vitro assays are quick, inexpensive and easy assays to run. Such assays generally use isolated molecules, such as cells, and can be run quickly and in large numbers, thereby increasing the amount of information obtainable in a short period of time. A variety of vessels may be used to run the assays, including test tubes, plates, dishes and other surfaces.

Various cell lines can be utilized for these assays, including cells specifically engineered for this purpose. Numerous cell lines and cultures are available for use, and they can be obtained through the American Type Culture Collection (ATCC), which is an organization that serves as an archive for living cultures and genetic materials (www.atcc.org). In certain embodiments, a cell may comprise, but is not limited to, at least one skin, bone, neuron, axon, cartilage, blood vessel, cornea, muscle, facia, brain, prostate, breast, endometrium, lung, pancreas, small intestine, blood, liver, testes, ovaries, cervix, colon, skin, stomach, esophagus, spleen, lymph node, bone marrow, kidney, peripheral blood, embryonic or ascite cell, and all cancers thereof.

Depending on the assay, culture of the cells maybe required. The cell is examined using any of a number of different physiologic assays. Such parameters include measurements of apoptosis, toxicity and cell death. These measurements are preformed using standard technqiues well known and used in the art. Alternatively, molecular analysis may be performed, for example, looking at protein expression, mRNA expression (including differential display of whole cell or polyA RNA) and others.

In further embodiments, a tissue may comprise a cell or cells to be transformed with nano-particles of the present invention or mixtures or combinations thereof. The tissue may be part or separated from an organism. In certain embodiments, a tissue may comprise, but is not limited to, adipocytes, alveolar, ameloblasts, axon, basal cells, blood (e.g., lymphocytes), blood vessel, bone, bone marrow, brain, breast, cartilage, cervix, colon, cornea, embryonic, endometrium, endothelial, epithelial, esophagus, facia, fibroblast, follicular, ganglion cells, glial cells, goblet cells, kidney, liver, lung, lymph node, muscle, neuron, ovaries, pancreas, peripheral blood, prostate, skin, skin, small intestine, spleen, stem cells, stomach, testes, ascite tissue, and all cancers thereof.

Additional in vivo assays involve the use of various animal models, including transgenic animals that have been engineered to have specific defects, or carry markers that can be used to measure the ability of a nano-shell of the present invention to effect different cells or tissues within the organism. Due to their size, ease of handling, and information on their physiology and genetic make-up, mice are a preferred embodiment, especially for transgenics. However, other animals are suitable as well, including rats, rabbits, hamsters, guinea pigs, gerbils, woodchucks, cats, dogs, sheep, goats, pigs, cows, horses and monkeys (including chimps, gibbons and baboons).

In such assays, one or more compositions of nano-particles of the present invention or mixtures or combinations thereof are administered to an animal, and the ability of the nano-particles of the present invention or mixtures or combinations thereof to alter cell proliferation, cell toxicity and/or apoptosis is compared to a similar animal not treated with the nano-particles of the present invention or mixtures or combinations thereof.

Treatment of these animals with nano-particles of the present invention or mixtures or combinations thereof will involve the administration of the nano-particles of the present invention or mixtures or combinations thereof, in an appropriate form, to the animal. Administration will be by any route that could be utilized for clinical or non-clinical purposes, including but not limited to intradermal, subcutaneous, intramuscular, intraperitoneal or intravenous injection. Specifically contemplated routes are systemic intravenous injection, regional administration via blood or lymph supply, or directly to an affected site.

### Therapeutic Methods

Unlike molecular fluorophores, nano-particles of the present invention or mixtures or combinations thereof are not generally subject to photo bleaching or photo induced damage. Since the nano-particles of the present invention or mixtures or combinations thereof resonance decays nonradiatively (with typical quantum efficiencies of a few percent), most of the energy due to optical absorption is converted into heat. Thus resonant illumination of highly absorptive nano-particles of the present invention or mixtures or combinations thereof can provide significant local heating to the microscopic environment of the nano-particles of the present invention or mixtures or combinations thereof. In illustration of this effect can be used to provide significant heat transfer to induce a phase transition in poly-N-isopropylacrylamide (NIPAAm), a polymer which undergoes an abrupt deswelling transition when raised above its lower critical solution temperature (LCST), nominally 45 .degree. C. (Sershen et al., 1999). When the copolymer is doped either homogeneously or heterogeneously with absorptive nano-particles of the present invention or mixtures or combinations thereof, the deswelling transition is induced by irradiation with light at the nano-particles of the present invention or mixtures or combinations thereof resonance wavelengths. This observation was verified against a control sample of copolymer without nano-particles of the present invention or mixtures or combinations thereof, to confirm that the weak residual absorption of the copolymer at the irradiation wavelength was insufficient to induce a temperature rise and the resultant deswelling transition. This local heating effect can be observed at relatively modest power levels using either continuous or pulsed laser sources, at power levels significantly less intense than those used in bioimaging applications. Therefore photo induced local heating of nano-particles of the present invention or mixtures or combinations thereof which are conjugated to antibodies which target cells (such as tumor or non-tumor cells) should lead to local, specific cell death. This type of inhibition can be useful in a variety of clinical conditions, for example but not limited to, tumors (malignant or benign) inflammatory responses or autoimmune diseases.

More generally, the nano-particles of the present invention or mixtures or combinations thereof may be used in an amount effective to kill or inhibit proliferation of a cancer cell. This process may involve contacting the cell(s), tissue or organism with the nano-particles of the present invention or mixtures or combinations thereof to produce a desired therapeutic benefit. This may be achieved by contacting the cell, tissue or organism with a single composition or pharmacological formulation that includes the nano-particles of the present invention or mixtures or combinations thereof and one or more agents, or by contacting the cell with two or more distinct compositions or formulations, wherein one composition includes nano-particles of the present invention or mixtures or combinations thereof and the other includes one or more agents.

The terms contacted and exposed, when applied to a cell, tissue or organism, are used herein to describe the process by which a therapeutic nano-particles of the present invention or mixtures or combinations thereof and/or another agent, such as for example a chemotherapeutic or radio-therapeutic agent, are delivered to a target cell, tissue or organism or are placed in direct juxtaposition with the target cell, tissue or organism. To achieve cell killing or stasis, the nano-particles of the present invention or mixtures or combinations thereof and/or additional agent(s) are delivered to one or more cells in an effective amount to kill the cell(s) or prevent them from dividing.

Various combination regimens of the nano-particles of the present invention or mixtures or combinations thereof and one or more agents may be employed. Non-limiting examples of such combinations are shown below, wherein a composition nano-shells is “A” and an agent is “B”: A/B/A, B/A/B, B/B/A, A/A/B, A/B/B, B/A/A, A/B/B/B, B/A/B/B, B/B/B/A, B/B/A/B, A/A/B/B, A/B/A/B, A/B/B/A, B/B/A/A, B/A/B/A, B/A/A/B, A/A/A/B, B/A/A/A, A/B/A/A, or A/A/B/A.

Administration of the nano-particles of the present invention or mixtures or combinations thereof to a cell, tissue or organism may follow general protocols for the administration of chemotherapeutics, taking into account the toxicity, if any. It is expected that the treatment cycles would be repeated as necessary. In particular embodiments, it is contemplated that various additional agents may be applied in any combination with the present invention.

Chemotherapeutic agents and methods of administration, dosages, etc. are well known to those of skill in the art (see for example, the “Physicians Desk Reference”, Goodman & Gilman's “The Pharmacological Basis of Therapeutics”, “Remington's Pharmaceutical Sciences”, and “The Merck Index, Eleventh Edition”, incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject. Examples of specific chemotherapeutic agents and dose regimes are also described herein. Of course, all of these dosages and agents described herein are exemplary rather than limiting, and other doses or agents may be used by a skilled artisan for a specific patient or application. Any dosage in-between these points, or range derivable therein is also expected to be of use in the invention.

The general method described herein is also useful where the targeted denaturation of proteins is desired. In such an application, the nano-particles of the present invention or mixtures or combinations thereof are directed to the proteins of interest by any of the targeting methods discussed. Local induction of hyperthermia will then effect denaturation. The denaturation primarily proceeds by the break-up of hydrogen bonds and other noncovalent interactions, although other harsher denaturation processes are possible depending upon the extent of heating. The denaturation may be effected either in vivo or in vitro.

Another therapeutic application, amenable to all the aforementioned schemes, is a highly localized, rapid induction of hyperthermia. The heat cycle could be commenced with a burst of exciting radiation, causing intense highly localized heating and very little heating to the surrounding bulk tissue. In this way, collateral damage is minimized. Such an approach could be used to remove non-cellular non-tissue material, such as coronary plaque. The general methodology has additional uses in the area of cosmetic enhancements. Intense localized hyperthermia can be used kill fat cells or to remove unsightly skin formations, among other potential cosmetic applications. Nano-particles of the present invention or mixtures or combinations thereof can be used as a secondary therapy to deliver heat and enable other, primary therapies. For instance, the level of heating in and of itself may be insufficient to cause cell death. However, the elevated temperatures may facilitate or accentuate other therapies such as chemotherapy or gene therapy.

### Diagnostic Methods

A variety of techniques for biomedical imaging with infrared diffusing light have been explored (Hebden, 1997). Time-gated methods, which involve the rejection of all photons except those traversing the sample via ballistic or quasi-ballistic trajectories, are conceptually straightforward; however, they favor the imaging of samples just a few millimeters in thickness. For biological samples of several centimeters in thickness, frequency domain approaches involving the detection of modulated laser light following its transmission through the tissue are particularly amenable. The resulting diffuse photon density waves (DPDW) are detected using demodulation schemes and analyzed and reconstructed using a range of methods (Jiang, et al, 1995; Li, et al, 1997; O'Leary, et al, 1995; Tromberg, et al., 1997). Sample-detector geometries for this type of imaging typically involve multiple source-detector arrays that maintain a constant source-detector distance around a cross section of the sample. Geometrics consisting of a single fixed light source and a scanned detector, which simplify data acquisition and reduce overall cost, are an extremely attractive simplification of this approach (Yang, et al, 1997).

### Nano-Particle-Based Imaging

The sensitivity of current infrared diffuse photon imaging methods is based on the contrast differences between the absorption and scattering coefficients of malignant and normal tissue. Typical differences in absorption and scattering coefficients vary from 33% to 66% and from 6% to 30%, respectively, from patient to patient (Tromberg, supra, 1997). These small differences determine image contrast, and therefore image resolution—typically just under 1 cm, again varying from patient to patient. There is therefore great interest in the use of specific contrast agents that would selectively target one type of tissue and enhance the contrast, and therefore the resolution, of the tomographic image. While this is a customary approach in biomedical imaging methods such as MRI and PET, there are very few contrast agents suitable for near infrared imaging. Only the tricarboxycyanine dyes, of which the best known member is indocyanine green (cardiogreen) have been approved for human use (Chance, 1993).

In contrast to indocyanine green, the nano-particles of the present invention or mixtures or combinations thereof have a million-fold enhancement in optical extinction: 10⁻¹⁵-10⁻⁶ cm² per molecule compared with 10⁻⁹-10⁻¹⁰ cm² per nano-particle (100 nm diameter). In addition, for indocyanine dyes, the optical extinction is almost purely absorptive, whereas nano-particles of the present invention or mixtures or combinations thereof can be fabricated either as scatterers or absorbers, to enhance either coefficient appropriately, as required.

### Nano-Emitter-Based Imaging

There has been considerable interest in the use of fluorescent dyes as contrast agents to differentiate diseased from normal tissue. Although dyes that excite and emit in the near infrared have been developed, which in principle would facilitate fluorescent imaging of diseased tissue deep in the body, issues such as low uptake and rapid photo-bleaching present significant problems regarding their utility. However, considerable interest remains, since the potential for correlating fluorescence lifetimes with tissue properties may provide important local information in the resulting fluorescence-based image (Paithankar, et al, 1997). Virtually all interest in this field has focused on molecular fluorophores, primarily due to their fast fluorescence lifetimes (typically 1-100 nanoseconds) which permit modulation techniques similar to those used in non-fluorescent infrared tomography.

Rare earth doped nano-emitters have several properties that contrast with molecular fluorophores. Due to the encapsulation of the emissive ions in a nano-particle of the present invention or mixtures or combinations thereof matrix, the local environment in which the nano-particle resides does not influence the nano-emitter fluorescence properties, as is the case for free molecular fluorophores. The concentration of rare earth emitters within silica nano-particles (typically a few percent) can be increased until the concentration is sufficient for self-quenching of the fluorescence to occur. Because of the high dopant density, the nano-emitters will exhibit much greater absorption than would be typical for isolated rare earth ionic species, as much brighter fluorescence.

In contrast to molecular fluorophores, rare earth ions have extremely long fluorescent lifetimes, often hundreds of microseconds in duration. This property eliminates the possibility of modulating the fluorescence of the nano-particles of the present invention or mixtures or combinations thereof by modulating the input beam of the excitation laser. However, the recent demonstration of ultrasonic modulation of scattered light in turbid media presents a useful method for modulating the nanoemitter fluorescence (L. V. Wang, 1998). With the addition of ultrasonic modulation, the frequency modulated detection strategy used in the nano-particle of the present invention or mixtures or combinations thereof experiments can be used in fluorescence imaging with rare earth nano-emitters.

Imaging based on the fluorescence of targeted nano-emitters should provide an increase in resolution relative to conventional infrared tomographic imaging methods. This is because the actual light source, that is, the nanoemitters themselves, will reside in or on the heterogeneity to be imaged. Since object resolution in turbid media scales linearly with the optical path length, the optical path length from scattered light originating within the sample is naturally shorter than the optical path length in a conventional transmissive imaging geometry. This could result in an average increase of resolution of a factor of two over transmissive imaging. Further increases in resolution will be obtainable due to the changes in μₐ and μₛ due to the presence of the nanoemitters themselves.

To eliminate shadowing effects, fluorescence imaging requires the excitation of the sample from a variety of directions, and multi-source, multidetector geometry. This type of experimental geometry lends itself to emission/transmission imaging, where reconstructed image quality can be improved by performing both emissive imaging as well as standard transmission imaging on the sample of interest, a strategy commonly applied to positron emission tomography (PET) (Tung, et al, 1992).

### Therapeutic Methods Using Nano-Shell, Nano-Rod, Nano-Rod Nano-Shell Nano-Particles

Under modest laser irradiation, nano-particles of the present invention or mixtures or combinations thereof can induce a significant temperature rise in their local environment. In a polyNIPAAm matrix, the local heating is sufficient to initiate a deswelling transition, corresponding to a temperature increase of approximately 8 degrees. This temperature increase has been measured directly in a solution of nano-particles of the present invention or mixtures or combinations thereof in water. In such experiments, a picomolar solution of nano-particles of the present invention or mixtures or combinations thereof with a resonance at 850 nm are irradiated on resonance with a 500 mW continuous wave Ti:Sapphire laser for a total of 20 minutes. After the first ten minutes of irradiation, a 9 degree temperature increase are generally observed. Heat loss to the surroundings prevented further heating of the sample upon continued irradiation. An aqueous control solution irradiated in the same manner showed no detectable temperature rise.

This local selective heating in the vicinity of nano-particles of the present invention or mixtures or combinations thereof can be applied for the thermal destruction of cancerous cells.

Methods, devices and compositions for the photo thermally modulated release of a chemical from a release medium are provided by the present invention. In a particular embodiment, methods, devices and compositions for the in vivo localized, photo thermally modulated release of a therapeutic agent, such as a drug, from an implanted medium are provided by the present invention. These methods, devices and compositions offer greater ability to localize heating and avoid potential damage to the surrounding tissue than is possible with existing methods and devices. The new composites, and their methods of use, are compatible with many types of therapeutic agents, including chemicals, drugs, proteins and oligonucleotides. The modulation is highly repeatable, allowing use of one device for many dosages.

One advantage of the present method and composite is the ability to locally change the temperature of a thermally responsive material by exposure to light targeted for absorption and conversion to heat by engineered nano-structures (nano-particles of the present invention or mixtures or combinations thereof). This allows implantation of a drug delivery device with many dosages, and provides for external control over the dosage profiles by regulating exposure to an appropriate light source.

In accordance with the present invention, a composition for modulated in vivo drug delivery to a subject in need thereof is provided. In certain embodiments the composition comprises a plurality of heat generating nano-particles of the present invention or mixtures or combinations thereof. Each of these nano-particles of the present invention or mixtures or combinations thereof has a non-conducting, semi-conducting or conducting core with an independently defined radius, a metal nano-shell and/or nano-rod adhering to the core and also having an independently defined thickness or dimension as previously described. The nano-particles of the present invention or mixtures or combinations thereof may be coated with or formed in bio-compatible polymer such as a hydrogel.

Another aspect of the present invention provides optically heatable nano-particles of the present invention or mixtures or combinations thereof suitable for use in the new compositions described above. The particles effectively convert incident electromagnetic radiation into heat energy when they are irradiated. The conversion of incident electromagnetic radiation into heat energy is optimized when the incident radiation is at the defined wavelength at which the particles' absorbance is at its maximum.

### Temperature Sensitive Polymers

Temperature sensitive polymers, such as N-isopropylacrylamide and elastin peptide polymers, were examined as candidates for a modulated drug delivery application, since they are capable of repetitive changes in polymer conformation (and thus permeability and rate of drug delivery) in response to relatively small changes in temperature. Photo thermally modulated drug delivery, wherein a device is implanted that allows the rate of drug delivery to be controlled by the application of electromagnetic energy to the device, is expected to be therapeutically beneficial in many cases, but especially so in insulin therapy. Near infrared light (800-1100 nm) passes through tissue with very little attenuation since there is very little absorption by the tissue. Thus, external access to an implanted device is possible and heating of the tissue surrounding the device is substantially avoided.

As stated above, N-isopropylacrylamide-co-acrylamide (NIPAAm-co-Aam) hydrogels are temperature-sensitive polymers whose lower critical solution temperatures (LCST) are only slightly above body temperature. When the temperature of the polymer is raised above its LCST, it undergoes a reversible phase transition, resulting in collapse of the NIPAAm-co-AAm hydrogel structure (A. S. Hoffinan et al. J. Contr. Rel. 4:213-222 (1986); and L. C. Dong et al. J. Contr. Rel. 4:223-227 (1986). The collapse forces materials held within the hydrogel matrix to be expelled into the surrounding solution (R. Yoshida et al. J. Biomater. Sci. Polymer Edn. 6:585-598 (1994). Pure NIPAAm hydrogels form a thick skin on their surface when they collapse, however, which greatly reduces transport of materials out of the hydrogels after the skin is formed (R. Yoshida et al. J. Biomater. Sci Polymer Edn. 6:585-598 (1994). Additionally, the LCST of unmodified NIPAAm is 32. degree. C., well below body temperature (J. H. Priest et al. Reversible Polymer Gels and Related Systems 350:255-264 (1987); and L. C. Dong et al. Reversible Polymer Gels and Related Systems 350:236-244 (1987)).

Copolymers formed of NIPAAm with the more hydrophilic AAm form a relatively thin surface layer, allowing soluble materials held within the hydrogel matrix to be more easily expelled into the surrounding solution during hydrogel collapse. NIPAAm-co-AAm hydrogels can have a LCST ranging from 32-65° C., depending on the amount of AAm included in the copolymer. A copolymer hydrogel consisting of 95% NIPAAm and 5% AAm has a LCST of approximately 40° C. (J. H. Priest, et al. Reversible Polymer Gels and Related Systems 350:255-264 (1987); and L. C. Dong et al. Reversible Polymer Gels and Related Systems 350:236-244 (1987). Hence, such a copolymer hydrogel is suitable for use in applications where it is desired to cause collapse of the hydrogel at temperatures only slightly above the normal core temperature of the human body.

Since it is not desirable to heat an implanted hydrogel directly, as this could cause thermal damage to the surrounding tissue, it is desirable to transfer energy to the hydrogel by some other means. IR light is one such means. NIPAAm-co-AAm hydrogels do not strongly absorb near IR light however. Thus, in order to achieve heating at the hydrogel with light that can pass harmlessly through surrounding tissue, light-absorbing nano-shells were embedded in the surface of aNIPAAm-co-AAm hydrogel. The extinction spectra of the composite over the near-IR spectrum is dictated by the nano-shells, while the phase transition characteristics of a NIPAAm-co-AAm copolymer with a LCST of approximately 40° C. are maintained in the composite.

In the preferred embodiment, a method of joining tissue comprises delivering nano-particles of the present invention or mixtures or combinations thereof that absorb light at one or more wavelengths to the tissue and, exposing the nano-particles of the present invention or mixtures or combinations thereof to light at one or more wavelengths that are absorbed by the nano-particles of the present invention or mixtures or combinations thereof. In the preferred embodiment, the light is laser light although it may alternatively be non-laser radiation. It is also preferred that the nano-particles used be nano-shell nano-particles, nano-rod nano-particles, and/or nano-rod nano-shell nano-particles. In a specific embodiment, the nano-particles are metal nano-shell nano-particles, nano-rod nano-particles, and/or nano-rod nano-shell nano-particles. Alternatively, the nano-particles are metal colloids, such as gold colloid or silver colloid. In another embodiment, the nano-particles may be fullerenes. In the preferred embodiment, all of the nano-particles are of the same composition; however alternatively, the nano-particles may be of more than one composition. In the preferred embodiment, the light is infrared light; in alternative embodiments, the light maybe visible or ultraviolet or any combination of infrared, visible, or ultraviolet light. In a specific embodiment, the light is red to near-infrared and is in the wavelength range of 600-2000 nm. In a preferred embodiment, the light is near-infrared light and is in the wavelength range of 700-1200 nm. Most preferably, the light is in the wavelength range of 750-1100 nm. The nano-particles of the present invention or mixtures or combinations thereof have dimensions of between 1 and 5000 nanometers. In the preferred embodiment, the nano-particles of the present invention or mixtures or combinations thereof have dimensions of between 1 and 1000 nanometers.

In a specific embodiment, at least a portion of the nano-particles of the present invention or mixtures or combinations thereof are mixed with one or more proteins. Specific embodiments of protein/nano-particle systems include nano-particles of the present invention or mixtures or combinations thereof mixed with albumin, fibrinogen, collagen, elastin, fibronectin, laminin, chitosan, fibroblast growth factor, vascular endothelial cell growth factor, platelet-derived growth factor, epidermal growth factor, or insulin-like growth factor or combinations thereof. Alternatively, at least a portion of the nano-particles of the present invention or mixtures or combinations thereof maybe mixed with one or more polymers. Specific embodiments of polymer/nano-particle systems include nano-particles of the present invention or mixtures or combinations thereof mixed with polyethylene, polyethylene glycol, polystyrene, polyethylene terephthalate, polymethyl methacrylate, or combinations thereof. In another embodiment, at least a portion of the nano-particles of the present invention or mixtures or combinations thereof is mixed with one or more polymers and one or more proteins. In a specific embodiment, at least a portion of the nano-particles of the present invention or mixtures or combinations thereof is bound to a chemical moiety. In a specific embodiment, at least a portion of the nano-particles of the present invention or mixtures or combinations thereof is bound to an antibody.

In another embodiment of the invention, a method of joining tissue to non-tissue material comprises delivering a first set ofiano-particles of the present invention or mixtures or combinations thereof that absorb light at one or more wavelengths to tissue, delivering a second set of nano-particles of the present invention or mixtures or combinations thereofthat absorb light at one or more wavelengths to non-tissue material, and exposing the first set of said nano-particles and the second set of nano-particles of the present invention or mixtures or combinations thereof to light at one or more wavelengths that are absorbed by the first set of nano-particles of the present invention or mixtures or combinations thereof and the second set of nano-particles of the present invention or mixtures or combinations thereof. In the preferred embodiment, the sets of nano-particles of the present invention or mixtures or combinations thereof are of the same composition. Alternatively, the sets of nano-particles of the present invention or mixtures or combinations thereof may be of different composition. In the preferred embodiment, the nano-particles of the present invention or mixtures or combinations thereof in the tissue and non-tissue absorb light at at least one common wavelength. Alternatively, they may absorb at different wavelengths. In the preferred embodiment, both sets of nano-particles of the present invention or mixtures or combinations thereof heat up simultaneously, thereby exhibiting the same heating profile. In alternative embodiments, the heating profiles may be different. In specific embodiments, one or both of the sets of nano-particles of the present invention or mixtures or combinations thereof are mixed with protein, polymer or a combination thereof. In the preferred embodiment, the light used is laser light, however, in an alternative embodiment, the light may be non-laser radiation. In a specific embodiment, the non-tissue is a medical device. In another specific embodiment, the non-tissues comprise engineered tissue.

In a specific embodiment of the present invention, a method for reducing wrinkles or other cosmetic defects such as stretch marks in tissue comprises delivering nano-particles of the present invention or mixtures or combinations thereof that absorb light at one or more wavelengths to the tissue and exposing said nano-particles of the present invention or mixtures or combinations thereof to light at one or more wavelengths that are absorbed by the nano-particles of the present invention or mixtures or combinations thereof. In other specific embodiments, methods for cosmetic or therapeutic laser resurfacing of tissue are used.

In another embodiment of the present invention, a method of heating tissue comprises delivering nano-particles of the present invention or mixtures or combinations thereof that absorb light at one or more wavelengths to the tissue and exposing the nano-particles of the present invention or mixtures or combinations thereof to light at one or more wavelengths that are absorbed by the nano-particles. The nano-particles of the present invention or mixtures or combinations thereof may be delivered to the tissue in a formulation containing a protein or polymer. In a specific embodiment of the invention, tissue is ablated by the method. In another embodiment, coagulation of blood is induced by the method.

In another embodiment of the invention, a method of joining non-tissue materials comprises delivering nano-particles of the present invention or mixtures or combinations thereof that absorb light at one or more wavelengths to one or more of the materials, exposing the nano-particles of the present invention or mixtures or combinations thereof to light at one or more wavelengths that are absorbed by the nano-particles. The nano-particles of the present invention or mixtures or combinations thereof may also be embedded within one or both non-tissue materials. In a specific embodiment, the non-tissue materials are polymers, such as polyethylene, polystyrene, polyethylene terephthalate, or polymethyl methacrylate. In this application, nano-particles are intended to absorb light and convert it to heat in order to raise the temperature of the material to near or above the melting temperature. This increases the mobility of polymer chains, allowing chains from the adjacent materials to become entangled and for the materials to become mechanically inter digitated, thus forming a union between the two materials. Ideally, the nano-particles of the present invention or mixtures or combinations thereof would absorb light at a wavelength where absorption of light by the polymer is low so that heating will be localized to the region where the nano-particles of the present invention or mixtures or combinations thereof are present. This can minimize the appearance of the joint between the two materials. Additionally, such an approach can minimize the size of the joint between two materials, which may be advantageous in microfabrication or other fabrication processes.

In a preferred embodiment, there is a method of joining tissue comprising the steps of delivering nano-particles of the present invention or mixtures or combinations thereof to the tissue, the nano-particles of the present invention or mixtures or combinations thereof having a light wavelength extinction maximum between 750 and 1100 nanometers, and exposing the nano-particles of the present invention or mixtures or combinations thereof to light at wavelengths between 750 and 1100 nanometers.

In a specific embodiment of the method, at least a portion of the nano-particles of the present invention or mixtures or combinations thereof is mixed with one or more proteins. In another specific embodiment, the one or more proteins is selected from the group consisting of albumin, fibrinogen, collagen, elastin, fibronectin, laminin, chitosan, fibroblast growth factor, vascular endothelial cell growth factor, platelet-derived growth factor, epidermal growth factor, or insulin-like growth factor and combinations thereof.

In an important embodiment of the present invention, the nano-particles of the present invention or mixtures or combinations thereof administered to an animal using standard methods. Animals that may be treated using the method of the invention include, but are not limited to humans, cows, horses, pigs, dogs, cats, sheep goats, rabbits, rats, mice, birds, chickens or fish.

A method to repair tissue for therapeutic applications has been developed. Such repair envisions the joining of tissue with other tissue or tissue with non-tissue material. The technique involves the use of nano-particles of the present invention or mixtures or combinations thereof which effect a localized heating when exposed to an excitation source which is typically light and more typically laser light, the localized heating effects tissue repair. The nano-particles of the present invention or mixtures or combinations thereof have dimensions of between 1 and 5000 nanometers. The excitation light used in typically NIR, although other excitation may be used such as the rest of the IR spectrum, UV, and VIS or combinations thereof. Typically, the light is in the wavelength range of 600-2000 nm; ideally, it is in the range of 750-1100 nm. The nano-particles of the present invention or mixtures or combinations thereof are ideally of nanometer-scale dimensions and are preferably up to 1000 nm in dimensions. Alternatively, they may be used which have dimensions of from greater than 1000 nm to 5000 nm. Well-known examples are colloids such as gold colloids and silver colloids. Alternatively, thenano-particles of the present invention or mixtures or combinations thereof may be nano-particles of the present invention or mixtures or combinations thereof such as those taught in U.S. application Ser. Nos. 09/779,677 and 09/038,377, and international application PCT/US00/19268, which are fully incorporated by reference as if expressly disclosed herein. The method typically involves the use of nano-particles of one composition; however, nano-particles of the present invention or mixtures or combinations thereof maybe used. If more than one composition of nano-particles of the present invention or mixtures or combinations thereof is used, it is typical for the different compositions to all absorb at at least one common wavelength; however, this is not absolutely necessary. As a result, the temporal heating profiles of the different nano-particles of the present invention or mixtures or combinations thereof may be the same or different. Typically, the temporal heating profiles are the same. The method may include targeting schemes to direct the nano-particles of the present invention or mixtures or combinations thereof to the desired location involving, for example, specific chemical interactions (e.g., antigen-antibody binding, etc.) or may consist of the simple delivery of the therapeutic reagents to the desired area. The direction or targeting of the therapy is primarily for the surface of the subject tissue; however, it may be targeted to other, interior sites. Treatment of the tissue surfaces may be accomplished by non-targeted delivery. Examples of non-targeted delivery include bathing tissue in nano-particle of the present invention or mixtures or combinations thereof suspensions, using a pipette or micro pipette to apply a nano-particle suspension to tissue, injecting a nano-particle suspension into tissue, painting nano-particles of the present invention or mixtures or combinations thereof onto tissues, or combining nano-particles with other ingredients such as one or more polymers and/or one or more proteins or combinations thereof. Examples include, but are not limited to albumin, fibrinogen, collagen, elastin, fibronectin, laminin, chitosan, basic fibroblast growth factor, or vascular endothelial cell growth factor, platelet-derived growth factor, epidermal growth factor, or insulin-like growth factor and directly placing this mixture on or between tissue surfaces. The invention encompasses the use of one or more other chemical entities or moieties to be used in conjunction with the nano-particles of the present invention or mixtures or combinations thereof. These species may have a complimentary or additional therapeutic or diagnostic utility. The nano-particles of the present invention or mixtures or combinations thereof may be chemically bound to these other components or may be delivered as a simple mixture with them. For example, the nano-particles of the present invention or mixtures or combinations thereof may be bound to antibody. The method of repair may involve only one type of nano-particle of the present invention or may involve more than one type of nano-particle of the present invention. For instance, one type of nano-particles of the present invention may be applied to one of the sites to be joined, while another type of nano-particles of the present invention may be applied to the other. Alternatively, nano-particles of the present invention or mixtures or combinations thereof may be applied to one or more of the sites to be joined. Whether the compositions are mixtures of nano-particles of the present invention or one type of nano-particle of the present invention, they may or may not contain other species, such as one or more types of polymers or one or more types of proteins, or both. It should be noted that the variations outlined above may be used in all the applications of the present invention, from tissue/tissue to tissue/non-tissue to non-tissue/non-tissue application. This is true notwithstanding that some of the specific examples given below may not expressly incorporate some or all of them.

Laser tissue welding refers to techniques by which tissues may be joined in response to exposure to light and the subsequent generation of heat. The goal of these techniques is (i) the rapid joining of tissues with high tensile strength across the union, (ii) union throughout the depth of the targeted tissue, (iii) a minimum of scar tissue formation, and (iv) minimal damage to surrounding tissue. These techniques may also be beneficial in a number of minimally invasive surgical techniques. In the preferred embodiment of the present invention laser excitation sources are used although alternative embodiments utilize non-laser excitation sources. Laser tissue repair is under investigation or in use in many surgical disciplines for procedures such as closure of skin wounds, vascular anastamosis, occular repair, nerve repair, cartilage repair, and liver repair. Currently, laser tissue repair is accomplished either through welding, apposing two tissue surfaces then exposing to laser radiation to heat the tissues sufficiently to join them, or through soldering, wherein an exogenous material such as a protein or synthetic polymer is placed between two tissue surfaces to enhance joining of the tissues upon exposure to laser radiation. The tissue repair and modification techniques described herein are optimally suited to the use of laser light due to the spectral properties (such as the tunability) of nano-particles of the present invention or mixtures or combinations thereof. However, they are also suited for non-laser based excitation.

Ideally, to maximize penetration of light through the depth of the wound and to minimize damage to surrounding tissue, one would prefer to use a laser light source that is not appreciably absorbed by tissues. This can be accomplished using NIR light, specifically in the wavelength region between 600-2000 nm, where penetration of light into tissue is maximal. Exposure to light at these wavelengths will not generate significant heating in tissues, and thus will not induce tissue damage. However, when light at these wavelengths interacts with nano-particles of the present invention or mixtures or combinations thereof designed to strongly absorb NIR light, heat will be generated rapidly and sufficiently to induce tissue welding. Because NIR wavelengths of light are highly transmitted through tissue, it is possible to access and treat tissue surfaces that are otherwise difficult or impossible.

The nano-particles of the present invention or mixtures or combinations thereof can be made to either preferentially absorb or scatter light by varying the size of the particle relative to the wavelength of the light at their optical resonance. Other materials may also be used. Organic conducting materials such as polyacetylene and doped polyanaline can also be used. Additional layers, such as a non-conducting layer, a conducting layer, or a sequence of such layers, such as an alternating sequence of conducting and non-conducting layers, can be bound to the nano-shell layer. The cores can be conducting, semi-conducting and/or non-conducting. The nature of the material affects the properties of the particles.

In the typical embodiment, the nano-particles of the present invention or mixtures or combinations thereof are not biodegradable but will tend to be cleared following administration by the reticuloendothelial system (RES). However, in some embodiments, it may be desirable to link the core, the metal shell or an intervening layer, using biodegradable materials such as a polyhydroxy acid polymer which degrades hydrolyticallyin the body so that removal of the particles after a period of time is facilitated.

A comprehensive investigation of the optical properties of metal nano-shells is reported by Averitt et al., 1997, as well as Averitt, et al., 1999. Quantitative agreement between Mie scattering theory and the experimentally observed optical resonant properties has been achieved. Based on this success, it is now possible to predictively design nano-particles of the present invention or mixtures or combinations thereof with the desired optical resonant properties, and then to fabricate the nano-shell with the dimensions and nanoscale tolerances necessary to achieve these properties (Oldenburg, et al., 1998).

### Nano-Particle Conjugated Antibodies

Because the metal nano-shells and/or nano-rods are grown using the same chemical reaction as gold colloid synthesis, the surfaces of nano-shells and/or nano-rods are virtually chemically identical to the surfaces of the gold nano-particles universally used in bioconjugate applications. The use of gold colloid in biological applications began in 1971, when Faulk and Taylor invented immunogold staining.

These particles may be subsequently aminated via reaction with aminopropyltriethoxysilane, thus allowing several options for antibody conjugation. Antibodies can be covalently immobilized to either hydroxylated or aminated nano-particle surfaces via a variety of chemical schemes, including carbodiimide chemistry, diisocyanate linkers, succinimidyl esters, etc. In addition, antibodies can be immobilized via polymer tethering chains. This can be accomplished with difunctional polyethylene glycol derivatives. This immobilization scheme may increase the biological activity of the immobilized antibodies by enhancing their mobility and thus their ability to interact with their target ligand. Efficiency of antibody immobilization can be determined with horseradish peroxidase (HRP) labeled antibodies. Activity of the nano-particle-conjugated antibodies can be assessed with HRP labeled antigens and by examining nano-particle binding to antigen-coated surfaces. Nano-particle binding to these surfaces can be quantitatively assessed by atomic force microscopy (AFM) and fluorescence. Results can be compared to ELISA measurements of the antigen surface concentration.

### Other Nano-Particle Systems

Other chemical or biochemical species may be used with nano-particles of the present invention or mixtures or combinations thereof in a mixed system to modify or otherwise enhance their properties for the specific application. For instance, they may be in a composition also containing proteins, polymers, or other chemical entities or moieties that aid in the delivery to the desired location. Mixtures of other components may be used with nano-particles. They may be mixed or bound to antigens to take advantage of the specificity of immunochemical binding. In addition to simple mixtures, these other species may be chemically bound as moieties to the nano-particles of the present invention or mixtures or combinations thereof.

The present invention relates to compositions and methods for synthesizing unique composite particles having homogeneous structures and defined wavelength absorbance maxima. The present compositions include a conducting, semi-conducting, magnetic, magnetically susceptible, or nonconducting inner layer or nano-particle core surrounded by a layer made of a conducting material, where in metal-metal nano-constructs the metal maybe the same or different. Also contemplated are unique methods for making the present compositions such that the resulting compositions can be tuned to absorb electromagnetic radiation maximally at wavelengths in the visible or infrared regions of the electromagnetic spectrum.

Particularly, the nano-particles of the present invention or mixtures or combinations thereof are not restricted to a single nano-particle core or single nano-shell or single nano-rod material; permutations of materials are made possible by the novel methodology disclosed herein for making the nano-particles of the present invention or mixtures or combinations thereof, including the bio-polymer coated nano-particles of the present invention or mixtures or combinations thereof. There is no requirement to use the nano-particles of the present invention or mixtures or combinations thereof in any given medium in order for them to exhibit their absorptive qualities; in fact, it is anticipated that such nano-particles of the present invention or mixtures or combinations thereof may find particular utility as surface treatments and coatings totally absent any surrounding medium. Because the core and shell material may be the same or different, any number of such permutations is made possible. The particles of the invention are also relatively uniform in size and shape by virtue of the methods of the invention used to construct them. Most importantly, while the nano-particles of the present invention or mixtures or combinations thereof may be much smaller than a wavelength of light, they are not limited in the thickness or dimensions of their metal nano-shells and/or nano-rods to account for the bulk properties of the nano-particles of the present invention or mixtures or combinations thereof. In fact, due to the one-atom-or-molecule-at-a-time approach to building the metal nano-shells and/or nano-rods disclosed by the present invention, the thickness of the metal nano-shells and/or nano-rods may be controlled from as low as atomic thicknesses.

The spectral location of the maximum of the plasmon resonance peak for this geometry depends sensitively upon the ratio of the nano-particle core radius to the dimension of the nano-shell (thickness) and/or nano-rods (length and circumference). The presence of a nano-particle core shifts the plasmon resonance to longer wavelengths relative to a solid nano-particle made exclusively of a single contiguous material. For a given core radius, a thin nano-shell or small nano-rods will have a plasmon peak that is shifted to longer wavelengths relative to a thicker nano-shell and/or larger nano-rods. It is to be emphasized that nano-particles of the present invention or mixtures or combinations thereof possess all of the same technologically viable optical properties as solid metal nano-particles in addition to this extremely important aspect of resonance tunability.

The present embodiments have wavelength absorbance maxima in the range of approximately 400 nm to 20 μm. The low wavelength end of the range is defined by the natural plasmon resonance of the nano-particles of the present invention or mixtures or combinations thereof. For any given nano-particle, the maximum absorbance depends upon the ratio of the thickness of the core to the nano-shell and/or nano-rod layer.

### Sensors

According to a preferred embodiment of the present invention, a chemical sensor includes of a thin film of resonant nano-particles of the present invention or mixtures or combinations thereof embedded in a semipermeable matrix, where the matrix is preferably semipermeable, more preferably permeable to an analyte of interest. The matrix is preferably transparent to the optical sampling wavelength and not Raman active at the Stokes shifts of interest. The optical sample wavelength may be 820 nm. Alternatively, the optical sampling wavelength may be any suitable laser wavelength. The matrix may be any suitable inorganic or polymeric material. One excellent candidate inorganic material for such a matrix material is mesoporous silica.

The optical sampling geometry can be as a layer deposited onto a reflective substrate exposed to incident light. Alternatively, the optical sampling geometry can be as a cladding layer in a waveguide structure, where the Raman excitation is a result of the evanescent wave of the guided optical mode propagating in that structure.

In either geometry, the analytes of interest are exposed to the semipermeable layer, diffuse through this layer and are adsorbed onto the surfaces of the embedded nano-particles of the present invention or mixtures or combinations thereof. The scattered light is modulated by the Stokes modes of the analyte molecules, and detection consists of spectral analysis of the scattered light using a standard dispersive geometry and lock-in based photodetection.

One direct advantage of Raman-based chemical sensing is its insensitivity to an H₂O solvent. This approach can be used in analytical scenarios such as VOCs (volative organic compounds) in groundwater samples or hydrocarbon mixtures in petroleum refinery or recovery. This geometry should also be amenable to vapor phase sampling of analytes. A further application is a biosensor, such as an immunoassay. The analyte may be any suitable analyte such discloses in the present references.sup.5,11 and/or in commonly assigned co-pending patent application Ser. No. 09/616,154, now U.S. Pat. No. 6,699,724 filed Jul. 14, 2000. The analyte may be a Raman-active chemical to be detected. Alternatively, the analyte may be a complex of a non-Raman active chemical to be detected with a Raman-active moiety.

### All-Optical Temperature Sensor

The active medium of this sensor consists of nano-particles of the present invention or mixtures or combinations thereof whose resonances are tuned to match the pump laser wavelength.

The nano-particles of the present invention or mixtures or combinations thereof can be functionalized with molecules that exhibit a strong Raman response. A variety of candidate molecules may be used, such as para-mercaptoaniline, which can be bound to the surface of the nano-particles of the present invention or mixtures or combinations thereof and which yields three strong Stokes modes. Alternatively the nano-particles of the present invention or mixtures or combinations thereof can be embedded in a medium 32 exhibiting a strong Raman response. Especially suited nano-particles of this invention are the so called sweat gum ball nano-rod nano-shell nano-particles described herein. The substantially asymmetric nature of the constructs should aid in these nano-particles having a strong Raman response.

For high temperature operation, a composite of semiconducting carbon nanotubes and nano-particles of the present invention or mixtures or combinations thereof can be used. Since the peak amplitudes of the corresponding Stokes and anti-stokes modes of the Raman-active molecules are related by the Boltzmann distribution, their ratio provides an optical readout of the ambient temperature of the sensor.

As for the chemical sensor described above, the optical sampling geometry can be as a layer deposited onto a reflective substrate exposed to incident light. Alternatively, the optical sampling geometry can be as a layer in a waveguide structure, where the Raman excitation is a result of the evanescent wave of the guided optical mode propagating in that structure.

This sensor can be designed for operation with a predetermined wavelength of light. According to some embodiments, the wavelength is 820 nm. Alternatively longer wavelengths, such as 1.06 μm maybe selected, to eliminate the resonant Raman response when semiconducting carbon nanotubes are used.

According to some embodiments, the resonant nano-particles are solid metal nano-particles and/or nano-particles of this invention or mixtures or combinations thereof. The shape of the nano-particles of the present invention or mixtures or combinations thereof maybe selected so as to adjust the wavelength of the resonance. Thus, contemplated shapes include spheroids, ellipsoids, needles, and the like. Further the metal nano-particles may be aggregated into multiparticle aggregates so as to adjust the wavelength of the resonance. Still further, the nano-particles of the present invention or mixtures or combinations thereof may be embedded in a matrix material that is capable of adjusting the wavelength of the resonance.

The wavelength of the resonance is preferably selected so as to provide surface enhanced Raman scattering. The wavelength may be controlled by controlling the geometry of the nano-particles of the present invention or mixtures or combinations thereof. According to some embodiments of the present invention, the nano-particles of the present invention or mixtures or combinations thereof are islands, such as may be formed as a stamped surface.

According to some embodiments of the present invention, the nano-particles of the present invention or mixtures or combinations thereof are arranged in a random array. Random as used herein denotes lacking X-ray scattering peaks with the range of length scales up to mesoscopic. According to some embodiments of the present invention, the nano-particles of the present invention or mixtures or combinations thereof are arranged in a regular array. Regular as used herein denotes possessing at least one X-ray scattering peak with the range of length scales up to mesoscopic.

According to some embodiments of the present invention, the nano-particles of the present invention or mixtures or combinations thereof are arranged in a two dimensional array. Alternatively, according to some embodiments of the present invention, the nano-particles of the present invention or mixtures or combinations thereof are arranged in a three dimensional array. Yet alternatively, the thin film may contain an arrangement of nano-particles of the present invention or mixtures or combinations thereof having a fractional dimension between two and three.

### Optical Device

It will be understood that the present optical device, such as a reflective device or a waveguide device, may be a component in an optical apparatus. Optical apparatuses that are contemplated include optical computing elements, holographic devices, optical correlators, optical phase conjugators, bistable memory devices, optical limiters, polarization filters, and infrared and visible light detectors.

When the optical device includes a reflective surface, the reflective surface may be a mirror. Alternatively, a reflective surface may a stack of dielectric thin films of alternating high and low refractive index. Such stacks are known that approach upwards of at least 90% reflectance. A spacer layer may be disposed between the reflective surface and the thin film containing the nano-particles of the present invention or mixtures or combinations thereof. The spacer layer may be formed of a dielectric material.

When the optical device includes a waveguide, the waveguide may include a dielectric layer supported on a metal layer. The thickness of the dielectric layer is preferably selected so as to support optical waves propagating parallel to the interface between the dielectric layer and the metal layer. The thin film layer containing the resonance nano-particles of the present invention or mixtures or combinations thereof may form a cladding layer of the waveguide. Methods of making the present optical devices include conventional microfabrication techniques such as known to one of ordinary skill in the art.

### Optical Coupling

The thin film is preferably optically coupled to the optical device. The optical coupling preferably occurs as a result of the geometry of the thin film with respect to the optical device. It will be understood that the preferred average distance between nano-particles of the present invention or mixtures or combinations thereof and a surface of the optical device may vary according to the wavelength of the maximum resonance of the nano-particles of the present invention or mixtures or combinations thereof, also termed herein resonant wavelength.

The average nano-particle distance to the nearest surface of the optical device is preferably up to a value on the order of the resonant wavelength. The average distance to the nearest surface is preferably determined as the average length of a vector oriented perpendicular to the outer surface of the optical device and extending from that outer surface to the center of mass of the nano-particles of the present invention or mixtures or combinations thereof.

The average nano-particle distance to a light directing surface as disclosed herein is likewise preferably up to a value on the order of the resonant wavelength. The average distance to the light directing surface is preferably determined as the average length of a vector oriented perpendicular to the light directing surface and extending from that light directing surface to the center of mass of the nano-particles of the present invention or mixtures or combinations thereof.

The light directing surface may be a metal surface in a waveguide. Alternatively, the light directing surface may be a reflective surface. Exemplary light scattering experiments described in U.S. Provisional Application 60/339,415 that were performed on gold nano-shells randomly deposited on a dielectric layer supported on a gold layer show a change in the scattering spectrum of the nano-shells due to coupling of light with the waveguide modes. Thus, these experiments demonstrated optically coupling of nano-particles of the present invention or mixtures or combinations thereof deposited on a waveguide structure with the waveguide. It is believed that these results extend to the nano-particles of the present invention or mixtures or combinations thereof embedded in the present matrix supported on the present optical device.

### Thin Film Formation

Forming the thin film preferably includes depositing a matrix material onto the optical device. The exposed surface of the optical device may be a metallic material. Alternatively, the exposed surface of the optical device may be a non-metallic material such as a dielectric material. The deposition may include spin-coating the matrix material. The matrix material maybe in the form of a fluid precursor during the deposition. The formation of the thin film then includes drying the fluid precursor so as to form the matrix as a solid that is preferably still gas or liquid permeable. Suitable inorganic materials include silica or other oxides that may be formed by a sol-gel process. Suitable polymeric materials include polyvinyl acetate (PVA).

The nano-particles of the present invention or mixtures or combinations thereof may be mixed into the fluid precursor prior to deposition. The nano-particles of the present invention or mixtures or combinations thereof can be successfully mixed by the present inventors into various polymers including PVA, polyvinylpropylene (PVP), polymethylmethacrylate (PMMA), and polydimethylsiloxane (PDMS). Further, methods for incorporating gold nano-particles in a silica sol-gel matrix are known to one of ordinary skill in the art. These methods are contemplated for incorporating the nano-particles of the present invention or mixtures or combinations thereof into inorganic oxide matrices. Alternatively, nano-particles of the present invention or mixtures or combinations thereof or other nanostructure may be formed on the optical device so as to form a composite structure, followed by depositing the fluid precursor to the composite structure.

According to some embodiments, forming the composite structure includes evaporating a solution a concentrated solution of the nano-particles of the present invention or mixtures or combinations thereof. A suitable exemplary method in which the optical device is a waveguide and the nano-particles of the present invention or mixtures or combinations thereof is described in the paper entitled “Light Interaction Between Gold Nano-shell Plasmon Resonance and Planar Optical Waveguides” contained in Provisional Application No. 60/339,415, which is incorporated herein by reference.

In an exemplary method, an approximately 200 nm thick layer of gold was sputter coated onto an indium tin oxide (ITO) coated glass slide. Self-Assembled Monolayers (SAM's) of a cationic polyelectrolyte PDDA (poly(diallyldimethylammonium chloride) and anionic sheets of an exfoliated synthetic clay (Laponite RD, a synthetic form of hectorite) were deposited on the gold surface to control the spacings to nominally nm precision between the gold surface and the nano-particles of the present invention or mixtures or combinations thereof. A sub monolayer of nano-particles of the present invention or mixtures or combinations thereof, with an average spacing of 200 nm and approximately 27% coverage (as determined by scanning electron microscopy) was deposited on the SAM's by evaporating 10-20 Ad of concentrated aqueous solution containing nano-particles of the present invention or mixtures or combinations thereof.

According to other embodiments, forming the composite structure includes mask-free lithographic formation of metal structures, such as metallic arrays. In an exemplary method, PDMS stamps were prepared in a standard way using an elastomer kit (Sylgard 184, Dow Corning). Diffraction gratings were purchased from Edmund Optics. Glass microscope slides were cleaned in piranha etch (7:3 v/v 98% H₂SO₄ :30% H₂O₂) for 1 hour, rinsed in ultrapure water (Milli-Q system, Millipore) and dried with a stream of filtered N₂. n-Propyltrimethoxysilane (PTMS), HAuCl₄, and K₂CO₃ were purchased from Sigrna-Aldrich Corp. and used as received. Silver plating was accomplished using a commercially available silver plating kit (HE-300, Peacock Laboratories Inc.) Scanning electron microscopy (SEM) was performed on a Phillips XL-30 ESEM. Atomic force microscopy (AFM) was performed on a Digital Instruments Nanoscope III.

Glass microscope slides were patterned with PTMS using stamps made from diffraction gratings and standard microcontact printing procedures. After the siloxane molecules had condensed on the surface (12 hours) the slides were exposed to a solution of SnCl₂ (Peacock Laboratories Inc.) for 5-10 seconds which activates the unstamped regions for metal reduction. Once activated the slides were washed with Milli-Q water and immediately exposed to silver or gold electroless plating solutions for a period of seconds or minutes until the metal had reduced onto the activated regions of the slides. Typical plating times ranged from 15 seconds to 1 minute. The silvering solution was used according to the provided instructions, while the gold solution was prepared by diluting 1 mL of a 1% HAuCl₄ solution in 100 mL H₂O and adding 25 mg K₂CO₃. After plating samples were rinsed well with water and dried with filtered nitrogen.

The present invention relates to adding nano-particles of the present invention or mixtures or combinations thereof to conducting polymers or other molecular systems that are vulnerable to photo-oxidation. In one embodiment, a triplet quencher (nano-particles of the present invention or mixtures or combinations thereof) is added to a polymer film. The nano-particles of the present invention or mixtures or combinations thereofpreferentially interact with the polymer triplet exciton, forming a relaxation pathway. By providing an additional de-excitation channel for the triplet exciton, it is possible to compete with singlet oxygen formation. Due to the central role of polymer triplet exciton T₁ in the photo-oxidation process, control over the triplet exciton dynamics leads to control over the photo-oxidation process. By providing an additional de-excitation channel for the triplet exciton, the rate of singlet oxygen formation and resultant photo-oxidation of the polymer can be reduced.

In order for the nano-particles of the present invention or mixtures or combinations thereof to interact with the polymer triplet exciton, the metal nano-shells are fabricated such that their plasmon resonance overlaps the conjugated polymer triplet exciton-ground state transition energy. In a preferred embodiment, the nano-particles of the present invention or mixtures or combinations thereof are fabricated such that their plasmon resonance wavelength corresponds to a wavelength for which the photon energy is equal to approximately 0.75-1.25 times, and more preferably 0.95 to 1.05 times, the conjugated polymer triplet exciton-ground state transition energy. The nano-particles of the present invention or mixtures or combinations thereof that are fabricated with a pre-selected plasmon resonance are sometimes referred to herein as “tuned” nano-particles of the present invention or mixtures or combinations thereof.

Preferred fabrication processes for nano-particles of the present invention or mixtures or combinations thereof are described herein or for dielectric nano-particle cores are described in U.S. Utility patent application Ser. No. 09/038,377, incorporated herein by reference.

Metal nano-shells suitable for use in the present invention include complete shells, hollow shells, partial shells (cups), and, in particular any nano-particle of the present invention or mixtures or combinations thereof. Additionally, it is contemplated that a reduction in photo-oxidation can be achieved in accordance with the present invention by including the “tuned” nano-particles of the present invention or mixtures or combinations thereof in proximity to the photo-oxidizable structure, i.e., without actually mixing the nano-particles of the present invention or mixtures or combinations thereof into the photo-oxidizable molecular system.

Once the nano-particles of the present invention or mixtures or combinations thereof are produced, they are then concentrated and transferred to an organic solvent that is compatible with conjugated polymer solution processing. Next, solutions of the conjugated polymer are prepared using appropriate solvents (e.g., chloroform or chlorobenzene). Small amounts of the nano-particle of the present invention or mixtures or combinations thereof solution are added to the conjugated polymer solution to reach the desired nano-particle of the present invention or mixtures or combinations thereof concentration. The resulting conjugated polymer/nano-particle of the present invention or mixtures or combinations thereof solution can then be used in standard device processing steps such as spin coating, drawing, extrusion, evaporative deposition, molding and the like. In one embodiment, it is preferred that the nano-particles of the present invention or mixtures or combinations thereof comprise between 10 and 50 percent of the volume fraction of the overall molecular system.

Because the typical conjugated polymer film thicknesses used in devices such as LEDs (100-200 nm) is similar to the diameter of some nano-particles of the present invention or mixtures or combinations thereof, the use of nano-shells in LEDs and other thin film applications will likely require the selection of smaller diameter nano-particles of the present invention or mixtures or combinations thereof. Because the metal-on-metal nano-shell and/or nano-rod nano-particles of this invention can be prepared having smaller and more uniform sizes, the thin films incorporating these metal-on-metal nano-shell and/or nano-rod nano-particles can be made thinner improving the films LED characteristics and utility. Additionally, because alternative LED fabrication techniques are currently being developed to improve device efficiency, such as employing additional, somewhat thicker, organic layers around the active conjugated polymer layer, next generation devices may not suffer from nano-particle of the present invention or mixtures or combinations thereof size limitations. For example, it may be possible to disperse nano-particles of the present invention or mixtures or combinations thereof into a thicker secondary layer in these LEDs. As will be noted, other conjugated polymer-based device structures such as conjugated polymer-based lasers use significantly thicker active regions and thus should be less sensitive to the size of the nano-particles of the present invention or mixtures or combinations thereof.

A possible variation of the present invention is in the field of “small organic molecule”-based electroluminescent devices such as hydroxy quinoline aluminum (AlQ3) devices, or organic light emitting devices (OLEDs). This technology has developed parallel to, and in many ways in competition with, conjugated polymer technology. It consists ofusing luminescent organic molecules as the active layer in optoelectronic devices. The organic molecules employed in these devices suffer a similar propensity to photo-oxidative degradation.

It should be understood that the present invention does not turn the photo-oxidation process off, rather it impedes its progress. Encapsulation techniques are currently being employed in conjugated polymer device fabrication that greatly reduce the rate of photo-oxidation by keeping oxygen out of the device. The addition of nano-particles of the present invention or mixtures or combinations thereof to conjugated polymer devices should be an excellent complement to encapsulation, yielding even longer device lifetimes.

When nano-particles of the present invention or mixtures or combinations thereof with resonances tuned to the polymer's triplet exciton energy are added to the conducting polymer, the resultant nano-particle-polymer composite exhibits dramatically reduced photo-oxidation rates with essentiallyno change in the luminescent properties, materials properties, or processing characteristics of the conducting polymer.

### Suitable Reagents for Use in the Invention

Suitable dielectrics for use in making the nano-particles, nano-shell nano-particles, nano-rod nano-particles, and/or nano-rod nano-shell nano-particles of this invention include, without limitation, any dielectric metal, semi-metal (or metalloid) and/or non-metal oxide and/or polymeric core. Non-limiting exemplary examples of metal oxide cores include alumina, silica, aluminosilicate, silicaaluminate, zirconia, titania, magnesia, or other similar oxides and mixtures or combinations thereof. Non-limiting exemplary examples of polymeric cores include polyesters, polyethers, polyimides, polyamides, polycarbonates, polyether-ketones (PEEK), polyphenyleneoxides (PPO), polyphenylenesulfide (PPS), polyvinylchloride (PVC), polychlorotrifluoroethylene, poly(p-phenyleneethylene, polystyrene polyethylene, polypropylene, polytetrafluoroethylene, or the like or mixtures or combinations thereof.

Suitable metasl for use in making the nano-particles, nano-shell nano-particles, nano-rod nano-particles, and/or nano-rod nano-shell nano-particles of this invention include, without limitation, any metal capable of forming nano-particles. Non-limiting exemplary examples include non-transition metals, transition metals, lanthanide metals, actinide metals, alloys thereofor mixtures or combinations thereof. Non-limiting exemplary examples of non-transition metals include aluminum (Al), silicon (Si), magnesium (Mg), calcium (Ca), strontium (Sr), barium (Ba), gallium (Ga), germanium (Ge), arsenic (As), selenium (Se), indium (In), tin (Sn), antimony (Sb), tellurium (Te), thallium (Tl), lead (Pb), bismuth (Bi), alloys thereof or mixture or combinations thereof. Non-limiting exemplary examples of transition metals include scandium (Sc), titanium (Ti), vanadium (V), chromium (Cr), manganese (Mn), iron (Fe), cobalt (Co), nickel (Ni), copper (Cu), zinc (Zn), yttrium (Y), zirconium (Zr), niobium (Nb), molybdenum (Mo), technetium (Tc), ruthenium (Ru), rhodium (Rh), palladium (Pd), silver (Ag), cadmium (Cd), hafnium (Hf), tantalum (Ta), tungsten (W), rhenium (Re), osmium (Os), iridium (Ir), platinum (Pt), gold (Au), mercury (Hg), alloys thereof, or mixtures or combinations thereof. Preferred metals include iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), alloys thereof or mixture or combinations thereof. More preferred metals include the noble metals ruthenium (Ru), rhodium (Rh), palladium (Pd), silver (Ag), osmium (Os), iridium (Ir), platinum (Pt), gold (Au), their alloys or mixtures and combinations thereof.

Suitable polymers for use in this invention include, without limitation, any polymeric material (homopolymers, copolymers, terpolymers or higher order multi-monomer polymers) or mixtures or combinations thereof into which one or more pore-forming agents can be introduced, dispersed, optionally force developed and later leached out of the composition. Non-limiting examples of such polymers include polymers of any polymerizable monomer such as polyolefins including polyalk-1-enes(polyethylene, polypropylene, copolymers of ethylene and propylene), vinyl aromatic polymers including polystyrenes, polysubstituted styrenes, polyvinyl pyridine, or the like, polyacrylates including polyacrylic acid, polymethacrylic acid, polymethacrylates, polyacrylated, polyesters such as PET, polylactides, and polyglycolides, polyurethanes, polymers containing one or more diene monomers including butadiene, isoprene, substituted butadienes or isoprenes, polyamides including polypeptides, polyimdes, polyacids, polyarylsulfides, polyarylsulfones, polycarbonates, EVAs, polyvinylacetates, polyvinylalcohols, polycaprolactones, polyanhydrides, polyesteramides, polyorthoesters, polydioxanones, polyacetals, polyketals, polycarbonates, polyorthocarbonates, polyphosphazenes, polyhydroxybutyrates, polyhydroxyvalerates, polyalkylene oxalates, polyalkylene succinates, poly(malic acid), poly(amino acids), poly(methyl vinyl ether), poly(maleic anhydride), chitin, chitosan, or non-carbon containing polymers such as polyphospoamides, polyalkyleneglycols such as polyethyleneglycol, polypropyleneglycol or mixtures thereof, or any other dissolvable or meltable polymer or copolymers, terpolymers, or higher poly-monomer polymers thereof or combinations or mixtures thereof.

Bio erodible polymers such as polyanhydrides or bulk erodible polymers such as polyorthoesters, including, without limitation, poly(1-lactic acid) (PILA), poly(d1-lactic acid) (PLA), poly(glycolic acid) (PGA), polycaprolactones, copolymers, terpolymer, higher poly-monomer polymers thereof, or combinations or mixtures thereof are preferred biocompatible, biodegradable polymers. The preferred biodegradable copolymers are lactic acid and glycolic acid copolymers sometimes referred to as poly(d1-lactic-co-glycolic acid) (PLG). The co-monomer (lactide:glycolide) ratios of the PLG polymers are preferably between about 100:0 to about 50:50 lactic acid to glycolic acid. Most preferably, the co-monomer ratios are between about 85:15 and about 50:50 lactic acid to glycolic acid. Blends of PLA with PLG, preferably about 85:15 to about 50:50 PLG to PLA, are also used to prepare polymer materials. PLA, PILA, PGA, PLG and combinations or mixtures or blends thereof are among the synthetic polymers approved forhuman clinical use. These copolymers offer the advantage of a large spectrum of degradation rates from a few days to years by simply varying the copolymer ratio of lactic acid to glycolic acid.

Hydrogel polymers for use in this invention include, without limitation, polyacrylamide polymers, polyacrylic acid polymers, polyethylene glycol (PEG) polymers, silicone polymers, protein polymers, other similar hydrogel polymers and mixtures or combinations thereof.

Suitable pharmaceutically active agents include, without limitation, any pharmaceutically active agent that can be either absorbed on the surface of any nano-particle of this invention or impregnated into a polymer-coating surrounding any nano-particle of this invention. Preferred agents includes anti-cancer agents, pathogenic agents for parasites, bacteria, virus, etc. Chemotherapeutic agents that may be used in combination with the present invention include, but are not limited to, 5-fluorouracil, bleomycin, busulfan, camptothecin, carboplatin, chlorambucil, cisplatin (CDDP), cyclophosphamide, dactinomycin, daunorubicin, doxorubicin, estrogen receptor binding agents, etoposide (VP16), farnesyl-protein transferase inhibitors, gemcitabine, ifosfamide, mechlorethamine, melphalan, mitomycin, navelbine, nitrosurea, plicomycin, procarbazine, raloxifene, tamoxifen, taxol, temazolomide (an aqueous form of DTIC), transplatinum, vinblastine and methotrexate, vincristine, or any analog or derivative variant of the foregoing. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, corticosteroid hormones, mitotic inhibitors, and nitrosoureas, hormone agents, miscellaneous agents, and any analog or derivative variant thereof.

### Experimental Section of the Invention

The following examples are offered by way of illustration and are not intended to limit the invention in any manner.

### EXAMPLE 1

### Silica Nano-Particle Core—Alloy Seed—Gold Nano-Shell

The present invention relates to improved metal oxide nano-particle cores having a nano-shell deposited or formed thereon, where the nano-shell comprises an noble metal alloy and where the resulting nano-shell nano-particles have improved optical characteristics. The method for making the nano-shell nano-particles improves structure, size, and optical properties of the nano-particles.

Materials

All chemicals were purchased from companies indicated in parenthesis. Formaldehyde (EMD Chemicals Inc. formally EM Science or Gibbstown, N.J.), sodium hydroxide (EMD Chemicals Inc. formally EM Science or Gibbstown, N.J.), ammonium hydroxide (30% NH₃ in water)(EMD Chemicals Inc. formally EM Science or Gibbstown, N.J.), sodium borohydride (EMD Chemicals Inc. formally EM Science or Gibbstown, N.J.), hydrochloric acid, and nitric acid (EMD Chemicals Inc. formally EM Science or Gibbstown, N.J.), potassium carbonate (J. T. Baker of Phillipsburg N.J.), hydrogen teterachloroaurate-(III) hydrate (Au 99.9%, Strem Chemicals, Inc. of Newburyport, Mass.), tetraethylorthosilicate (TEOS) (Sigma-Aldrich, Inc. of St. Louis, Mo.), terakis(hydroxoymethyl)phosphonium chloride (THPC) (Sigma-Aldrich, Inc. of St. Louis, Mo.), 3-aminopropyltrimethoxysilane (Sigma-Aldrich, Inc. of St. Louis, Mo.), ethanol (McCormick Distilling Co.), silver nitrate (Mallinckrodt of Hazelwood, Mo.). All the chemicals were used as received without further purification. Highly pure water was purified to a resistance of 10 MΩ (Milli-Q Reagent Water System; Millipore Corporation) and filtered through 0.22 μm filter to remove any aggregated impurities. All glassware were cleaned in an aquaregia (3:1, HCl:HNO₃) solution first then cleaned in base bath (saturated KOH in isopropyl alcohol) and rinsed in Milli-Q water prior to use.

Characterization Methods

All products were examined by ultraviolet-visible (UV-vis) spectroscopy, field emission scanning electron microscopy (FE-SEM), transmission electron microscopy (TEM), dynamic light scattering (DLS) and energy dispersive X-ray (EDX).

For the optical properties, UV-vis spectra were obtained using a Carry 50 Scan UV-visible spectrophotometer over awavelength range from 300 to 1100 nm. All samples were centrifuiged and re-dispersed and diluted in Milli-Q water and transferred into a quartz cell with optical glass windows.

For the morphology and distribution of silica core gold nano-shell nano-particles, FE-SEMs were performed using a JSM 6330F (JEOL) instrument operating at 15 kV and TEMs were carried out using a JEM-2000 FX electron microscope (JEOL) at accelerating voltage 200 kV equipped with EDX (Link analytical EXL, Oxford) analyzer. In order to get better FE-SEM images, the nano-shell nano-particles were deposited on a gold-coated silicon wafer and completely dried prior to coating with carbon. The samples were then coated with a carbon film to improve electrical conductivity. For TEM analyses, the samples were deposited on 300 mesh Holey carbon-coated copper grid and dried before examination.

In order to measure the particle diameters, DLS analyses were performed using an ALV-5000 Multiple Tau Digital Correlation instrument operating using a light source at 514.5 nm wavelength and a fixed scattering angle of 90°. The sample diameters were compared to the data from TEM and FE-SEM analyses for the consistency.

Preparation of Amine-Functionalized Silica Nano-Particles

The preparation scheme used in this invention is a modification of the well-known Stöber method. (See Stöber, W.; Fink, A.; Bohn, E. J. Colloid Interface Sci. 1968, 26, 62.) Ammonium hydroxide (13.4 mL) was mixed with 100 mL of absolute ethanol in a 500 mL two-necked round bottom flask. The mixture was stirred for 15 minutes and tetraethylsilicate or tetraethoxysilane (TEOS) was quickly added. The particle formation was observed in 30 minutes due to the color change of the mixture from colorless to milky white. From the FE-SEM and TEM images as well as DLS data, the particle diameters were ˜350 nm spherical shape overall (data not shown). 3-aminopropyltrimethoxysilane (APTMS) (0.16 mL) was then added to the previously prepared 100 mL of silica nano-particles in a 250 mL two-necked round bottom flask with stirring. The mixture was vigorously stirred for 24 hours at room temperature and 10 mL of ethanol was added drop-wise during the reflux step to enhance covalent bonding of APTMS onto the silica nano-particles, while it was heated to 85° C. for 1 hour. (See, e.g., Waddell, T. G.; Leyden, D. E.; DeBello, M. T. J. Am. Chem. Soc. 1981, 103, 5303. van Blaaderen, A.; Vrij, A. J. J. Colloid Interface Sci. 1993, 156, 1.) The solution was centrifuged at 3000 rpm (revolution per minute) for 1 hour and redispersed in 100 mL of ethanol twice. There were no considerable differences between the unfunctionalized silica nano-particles and ftnctionalized silica nano-particles from the FE-SEM, TEM, or DLS results.

THPC Gold-Silver Alloy Seed Preparation

Gold-silver alloy seeded nano-particles were prepared using terakis (hydroxoymethyl) phosphonium chloride (THPC). This THPC gold-silver alloyprocedure is amodification of the Duff et al. method for forming nano-shells on silica nano-particles. (See, e.g., Duff, D. G.; Baiker, A. Langmuir 1993, 9, 2301. Duff, D. G.; Baiker, A. Langmuir 1993, 9, 2310.) 1 mL of sodium hydroxide (1 mol) and 2 mL of a THPC solution (12 μL of 80% THPC in 1 mL of Milli-Q water) were mixed with 100 mL of Milli-Q water in a 250 mL flask. The reaction mixture was vigorously stirred for 5 minutes, after that 2 mL of 1 wt. % aqueous AgNO₃ and 1 wt. % HAuCl₄.3H₂O were added quickly to the mixture. The mixture was stirred for about 30 more minutes. The color of the solution changed very quickly from colorless to dark reddish yellow indicating the formation of gold-silver alloy seeded nano-particles having a diameter of between about 4 and about 6 nm. This solution was stored in the refrigerator for three days prior to use.

Gold Nano-Shell Growth

To grow the gold layer on the THPC alloy seed-attached silica nano-particles, the prepared K-gold solution 8 ml was placed in a 25 ml beaker with stir bar and added varying amount of THPC gold attached silica nano-particles from 0.2 to 2 mL to produce different thickness of gold shells. The mixture was stirred at least 10 minutes and added 0.02 mL of formaldehyde to reduce K-gold solution. The color change of solution occurred from colorless to blue, green, and yellowish green dependent on the shell thickness. The gold nano-shells were centrifuged and re-dispersed in Milli-Q water to remove un-reacted chemicals until use.

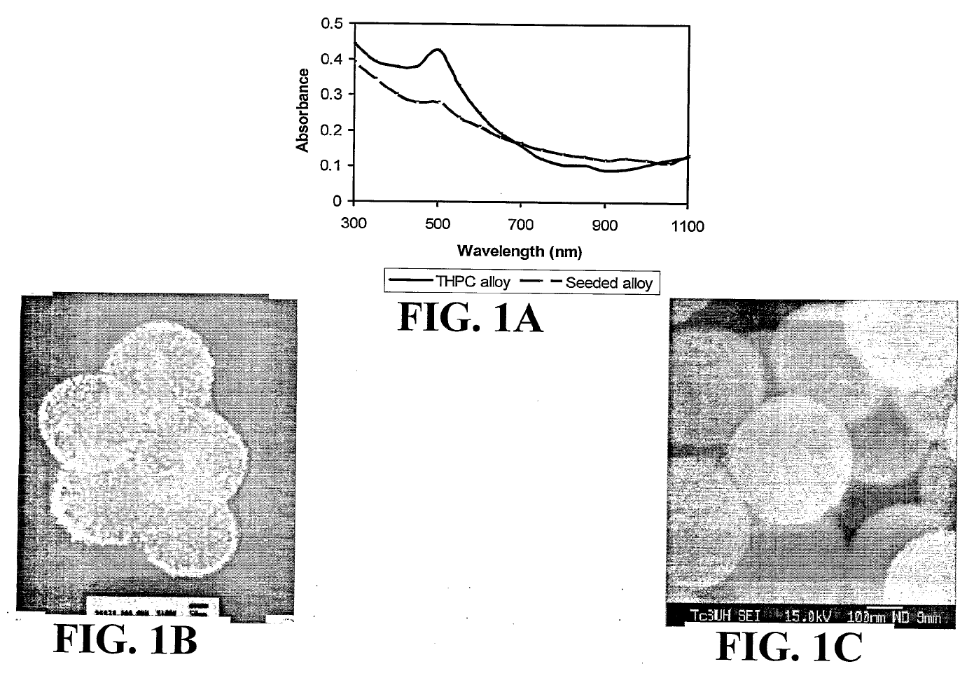

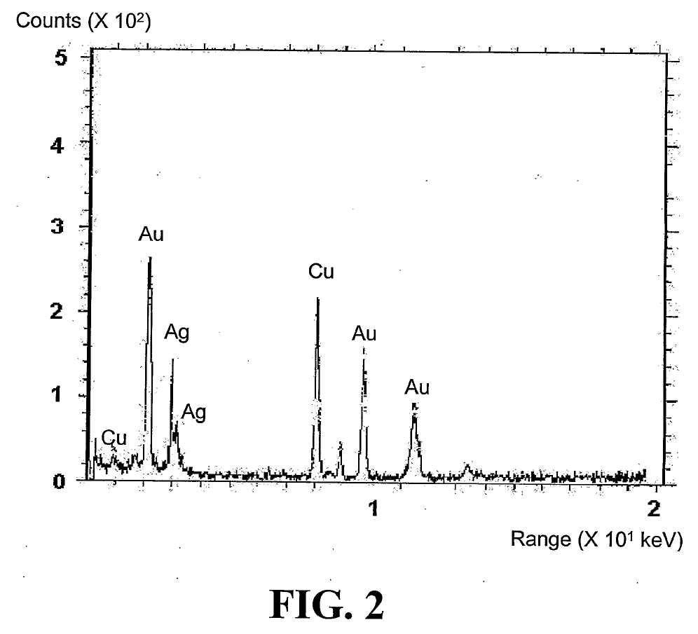

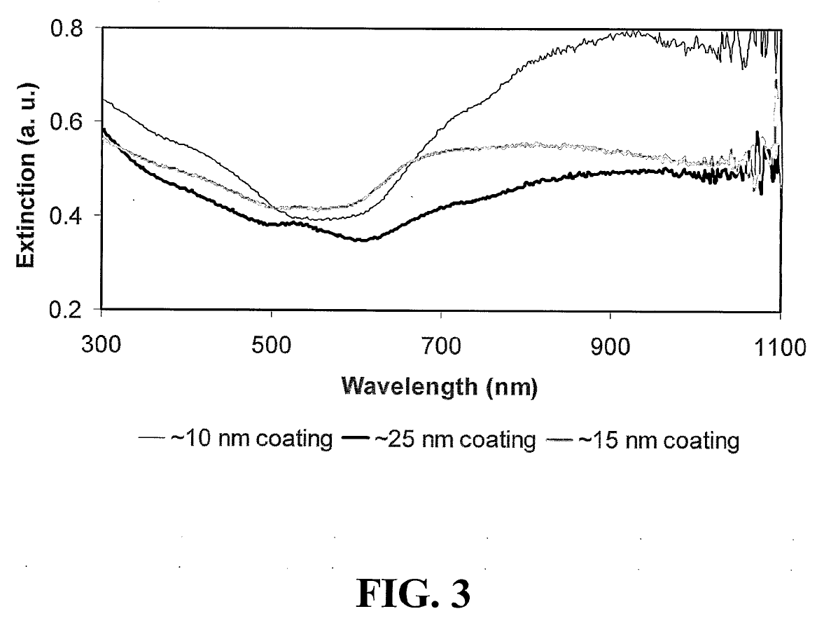

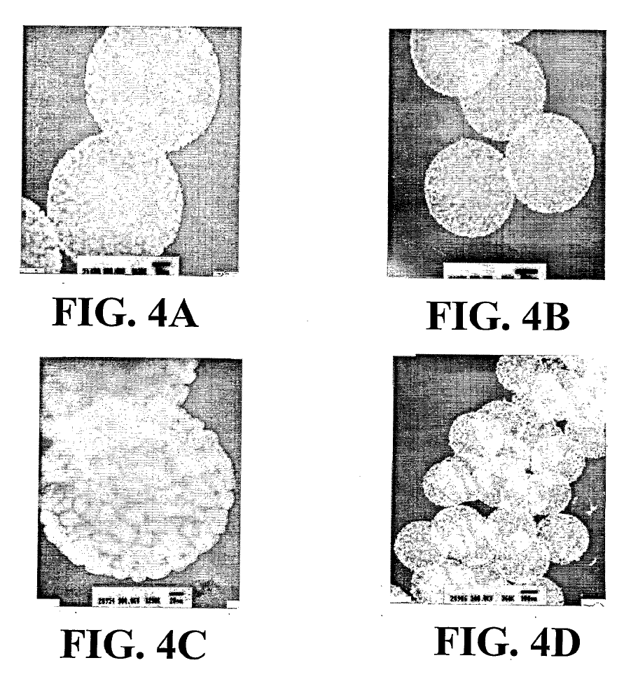

Referring now to FIGS. 1A-C, THPC gold-silver alloy seeded on silica nano-particles is shown. FIG. 1A shows UV-vis spectra of pure THPC alloy seeds and deposited alloy seeds on silica nano-particles. FIG. 1B shows a TEM image of alloy seeds on silica nano-particles, and FIG. 1C shows an FE-SEM image of alloy seeds on silica nano-particles. Referring now to FIG. 2, an EDX spectrum of alloy seeds deposited on silica nano-particles is shown. Referring now to FIG. 3, UV-vis spectra of alloy seed-gold nano-shell nano-particles is shown. Referring now to FIG. 4, TEM images of THPC alloy seed-gold nano-shell nano-particles are shown. From the Figures it is clear that the alloy seeded nano-shell nano-particles have a more uniform and thinner gold coating and the UV-vis spectra show plasmon resonance in the near infrared.

### EXAMPLE 2

### Gold, Silver, and Gold-Silver Alloy Nano-Shell Growth

Materials

All chemicals were purchased from the following companies below; Sodium hydroxide, formaldehyde, ammonium hydroxide (30% NH₃), sodium citrate dihydrate, nitric acid, hydrochloric acid (EM Science), potassium carbonate (J. T. Baker), tetraethylorthosilicate, terakis(hydroxoymethyl)phosphonium chloride, 3-aminopropyltrimethoxysilane (all from Aldrich) hydrogen teterachloroaurate-(III) hydrate (Strem), ethanol (Mckormick Distilling Co.), silver nitrate (Mallinckrodt). All the chemicals were used as received without further purification. Water was purified to a resistance of 10 MΩ (Milli-Q Reagent Water System; Millipore Corporation) and filtered using 0.2 μm filter to remove any impurities. All glassware and equipment used in the experiment were cleaned in an aqua regia solution (3:1, HCl:HNO₃) first then cleaned in base bath (saturated KOH in isopropyl alcohol) and rinsed in Mill-Q water prior to use.

Attachment of Colloidal THPC Gold Nanoparticles to Amine-Functionalized Silica Particles

This is the modification of Westscott et. al. method to make gold seed attached onto silica core particles. (Westscott, S. L.; Oldenburg, S. J.; Lee, T. R.; Halas, N. J. Langmuir 1998, 14, 5396.) The TBPC gold nanoparticles were deposited onto the silica particles by mixing THPC gold nanoparticles and amine functionalized silica nanoparticles for overnight. An lml of amine-functionalized silica particles dispersed in ethanol was placed into a 50 ml centrifuge tube with an excess ofthree-day aged THPC gold nanoparticles. The mixture was shaken for a couple of minutes and left for overnight to attach gold seeds to silica particles by self-assembly. The mixture was then centrifuiged for 1 h at 3000 rpm and the dark red-colored precipitate were redispersed in 50 ml of water. We briefly sonicated it for 5 min and centrifuged it again for 60 min. The solution showed very light red color after the precipitate was redispersed in 50 ml water.

K-Gold, Silver, and Gold-Silver Alloy Preparation

K-gold solution was prepared using 0.025 g potassium carbonate (K₂CO₃) in 100 ml water. The mixture was stirred for at least 15 min to dissolve K₂CO₃ completely and added 2 ml of 1% HAuCl₄.3H₂O. The color of solution was changing yellow into colorless within 40 min. K-silver and alloy solution were prepared at the same manner.

Gold, Silver, and Alloy Nano-shell Growth

To grow the gold layer on the THPC gold attached silica nanopartciles, the prepared K-gold solution 8 ml was placed in a 25 ml beaker with stir bar and added varying amount of THPC gold attached silica nanoparticles from 0.2 to 2 ml to produce different thickness of gold shells. The mixture was stirred at least 10 min and added 0.02 ml of formaldehyde to reduce K-gold solution. The color change of solution occurred from colorless to blue, green, and yellowish green depending on the shell thickness. The gold nanoshells were centrifuiged and redispersed in Milli-Q water to remove unreacted chemicals until use. Other shells such as silver and alloy shells were prepared at the same manner, but the color changes were a little different from each core shell particles.

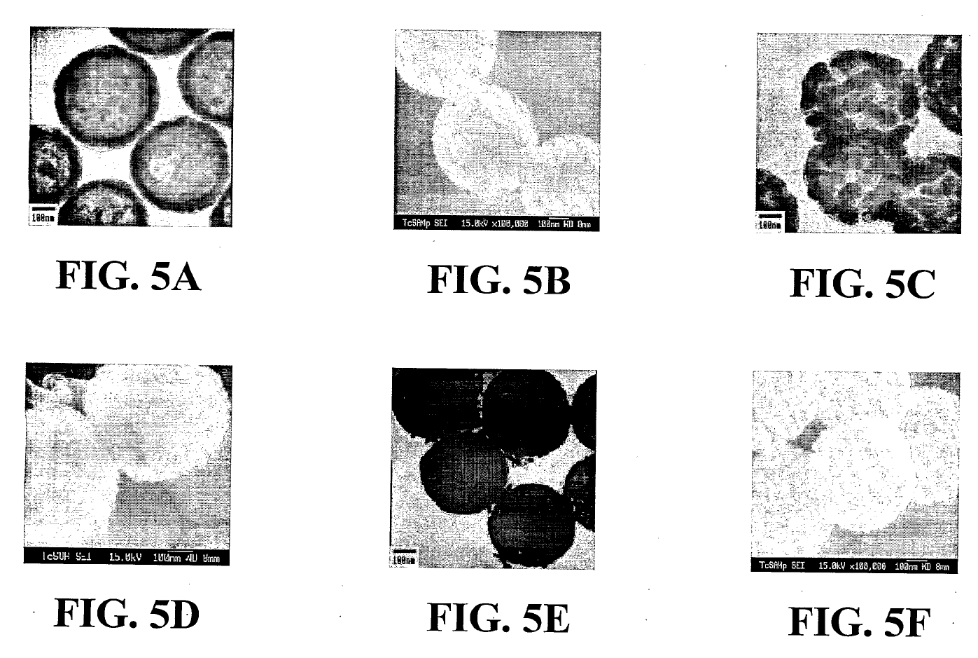

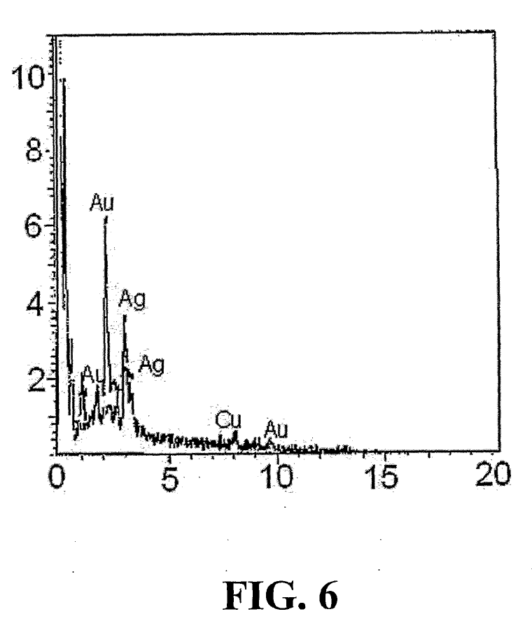

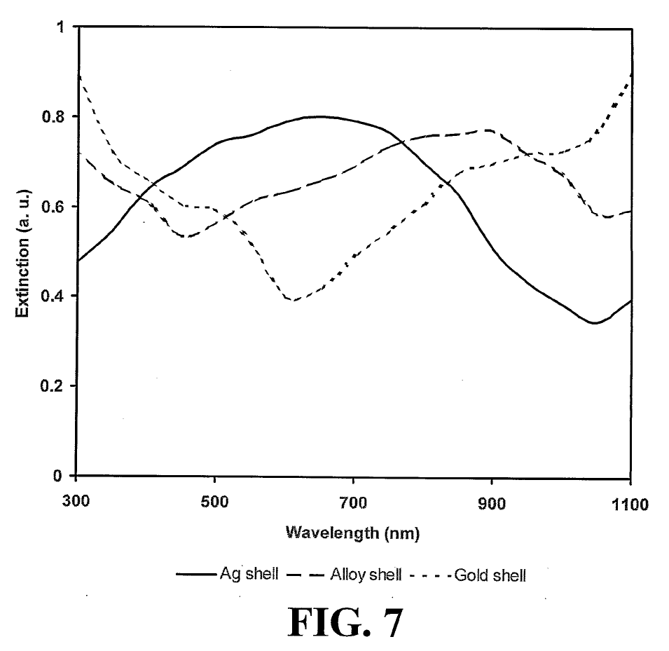

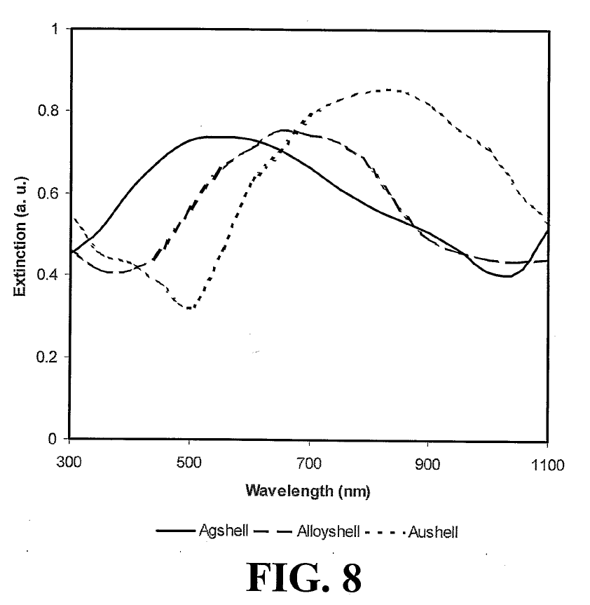

Referring now to FIGS. 5A-F, TEM and SEM images of gold, silver, gold-silver alloy nano-shells having a diameter of ˜350 nm silica core nano-particles and a nano-shell thickness of ˜30 nm are shown. Looking at FIG. 5A, a TEM image of gold nano-shell nano-particles is shown. Looking at FIG. 5C, a TEM image of silver nano-shell nano-particles is shown. Looking at FIG. 5E, a TEM image of alloy nano-shell nano-particles is shown. Looking at FIG. 5B, a FE-SEM image of gold nano-shell nano-particles is shown. Looking at FIG. 5D, a FE-SEM image of silver nano-shell nano-particles is shown. Looking at FIG. 5F, a FE-SEM image of alloy nano-shell nano-particles is shown. FIG. 6 shows an EDX spectrum showing ˜15 nm gold-silver alloy shells with ˜350 nm silica cores. FIG. 7 shows UV-vis spectra of gold, silver, and gold-silver alloy shells having a diameter of ˜350 nm silica cores and a shell thickness of ˜15 nm. FIG. 8 shows UV-vis spectra of gold, silver, and gold-silver alloy shells having a diameter of ˜350 nm silica cores and a shell thickness of ˜30 nm.

### EXAMPLE 3

### Silver Core Nano-Particles Having a Gold Nano-Shell

The present invention relates to metallic nano-particles such as silver nano-particles having deposited thereon a shell of a noble metal such as gold. These metal-core-noble-metal-nano-shell nano-particles have improved optical properties for use in optical electronics such as OLED displays and improved drug-delivery systems for the site specific delivery of drugs for cancer treatments or other diseases where the nano-particles can be directed to a body site and irradiated resulting in thermal death of cells in the body site or delivery of drugs to treat symptoms or ameliorate symptoms of diseases. For non in vivo use the nano-particles can be used for light induced release or absorption of a desired material.

Materials

Sodium citrate dihydrate, nitric acid, hydrochloric acid (EM Science), potassium carbonate (J. T. Baker), hydrogen teterachloroaurate-(III) hydrate (Strem), silver nitrate (Mallinckrodt) were purchased from indicated companies. All the chemicals were used as received without purification. Water was purified to a resistance of 18 MΩ (Academic Milli-Q Water System; Millipore Corporation) and filtered using 0.22 μm filter. All glassware used in the experiment were cleaned in an aquaregia solution (3:1, HCl:HNO₃) first and cleaned in base bath (saturated KOH in isopropyl alcohol) prior to use.

Characterization Methods

All the nano-particles were characterized by ultraviolet-visible (UV-vis) spectroscopy for the optical properties, by field emission scanning electron microscopy (FE-SEM) and transmission electron microscopy (TEM) for the morphology, by dynamic light scattering (DLS) for the diameters of nano-particles, by energy dispersive X-ray (EDX) for elemental compositions.

First, JSM 6330F (JEOL) FE-SEM and JEM-2000 FX electron microscope (JEOL) TEM was used to observe the morphology and particle distribution of the nano-particles. FE-SEM was operated at accelerating voltage 15 kV and equipped with a setup for elemental analysis by EDX (Link ISIS software series 300, Oxford Instruments) and TEM was accomplished at accelerating voltage 200 kV. The samples were placed on Formvar-coated copper grid and dried at room temperature overnight before the FE-SEM and TEM analysis. The sample for FE-SEM was then coated with carbon sputtering machine in order to get high-resolution images. The samples were examined by FE-SEM images (magnification 20,000-150,000X) and TEM images (100,000-500,000X) to show the morphology and overall uniformity of nano-particles on the surface.

ALV-5000 Multiple Tau Digital Correlation instrument operating at a light source 514.5 nm wavelength and a fixed scattering angle of 90° C. was used to measure nanoparticle sizes for the DLS measurements. The sample sizes were compared to the data from TEM and FE-SEM for the consistency.

A Cary 50 Scan UV-visible spectrometer was used over the range from 300-1100 nm wavelength to observe optical properties of nano-particles. All samples were centrifuged and redispersed in Milli-Q water to adjust concentration of each samples and transferred into a UV cell to measure the optical properties.

Preparation of Silver Nano-Particles

This is a modification of the well known Lee and Meisel method to make variable sizes of silver nano-particles. (Langmuir 2001, 17.574-577. Journal of Colloid and Interface and Science, 1983, 93, 545-555; J. Phys. Chem. 1982, 86, 3991.) 200 mL of a 10⁻³M AgNO₃solution was heated to boiling, and added 4 mL of a 1% trisodium citrate as soon as it reaches boiling. The mixture was kept stirring and boiling for 45 minutes to get homogeneous silver nano-particles. Other sizes of silver nano-particles were prepared from different concentrations of silver nitrate with constant amount of sodium citrate.

K₂CO₃-Gold (K-Gold) Preparation

To make K-gold solution, 0.05 g of potassium carbonate (K₂CO₃) in 200 mL Milli-Q water were stirred for at least 15 min to dissolve K₂CO₃ completely and added 4 mL of 1 wt % HAuCl₄.3H₂O. The color of solution changes from yellow to almost colorless within 40 min.

Gold-Coated Silver Nano-Particles by Self-Assembly

In order to grow the gold layer on the silver nanopartcle cores, the prepared K-gold solution 8 mL was placed in a 25 mL beaker with stir bar and added prepared silver nanoparticle cores (0.5 to 6 mL) to produce different thickness of gold layers. The mixture was kept stirring at least 10 minutes and changed colors from light yellow to greenish blue. The mixture was left for at least one day to get complete coating and centrifuged at 2500 rpm (revolution per minute) for 1 hour using RC-3B Refrigerated Centrifuge (Sorvall Instruments) and redispersed in Milli-Q water for the analysis.

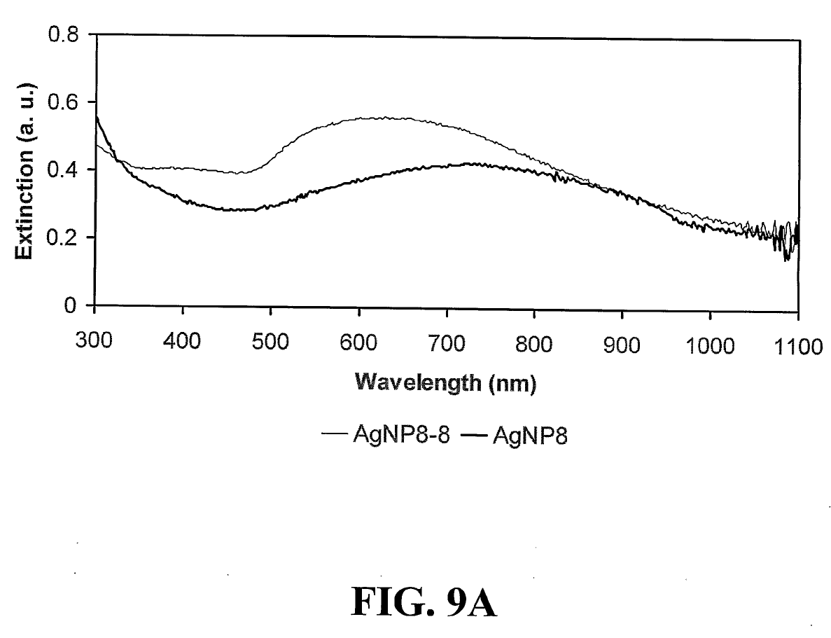

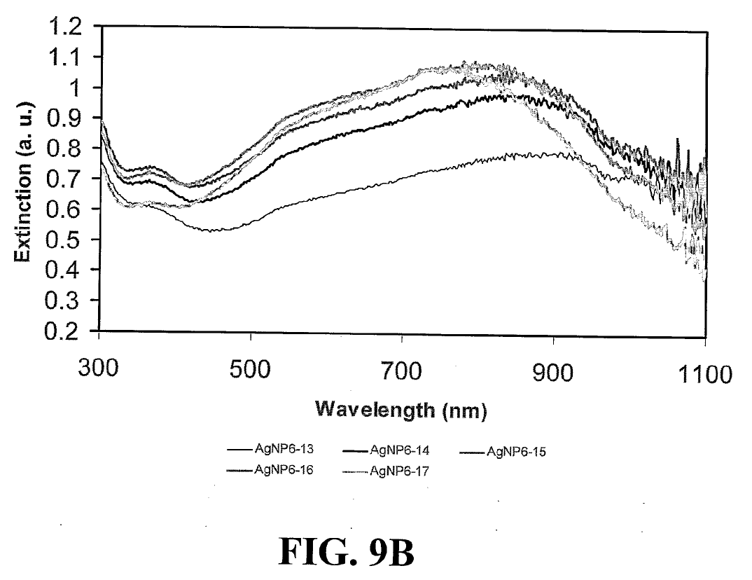

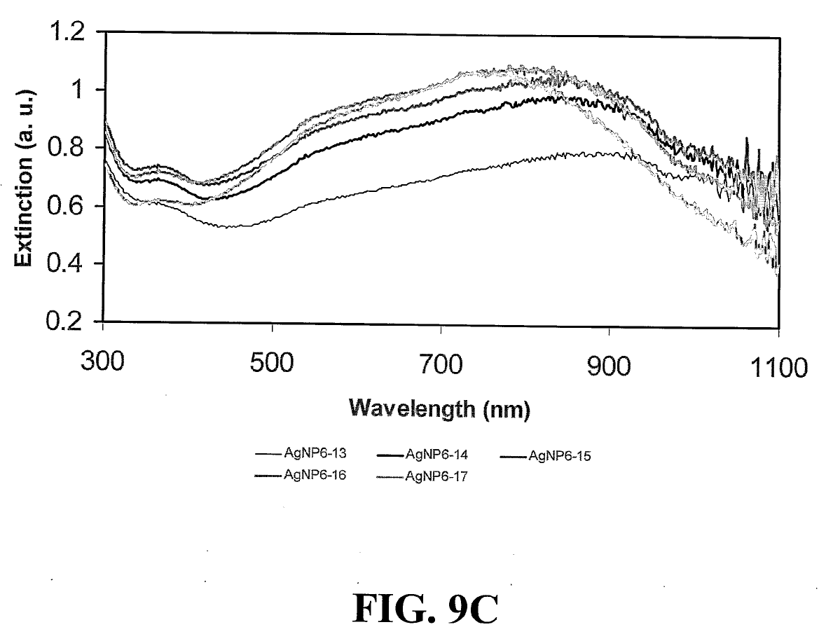

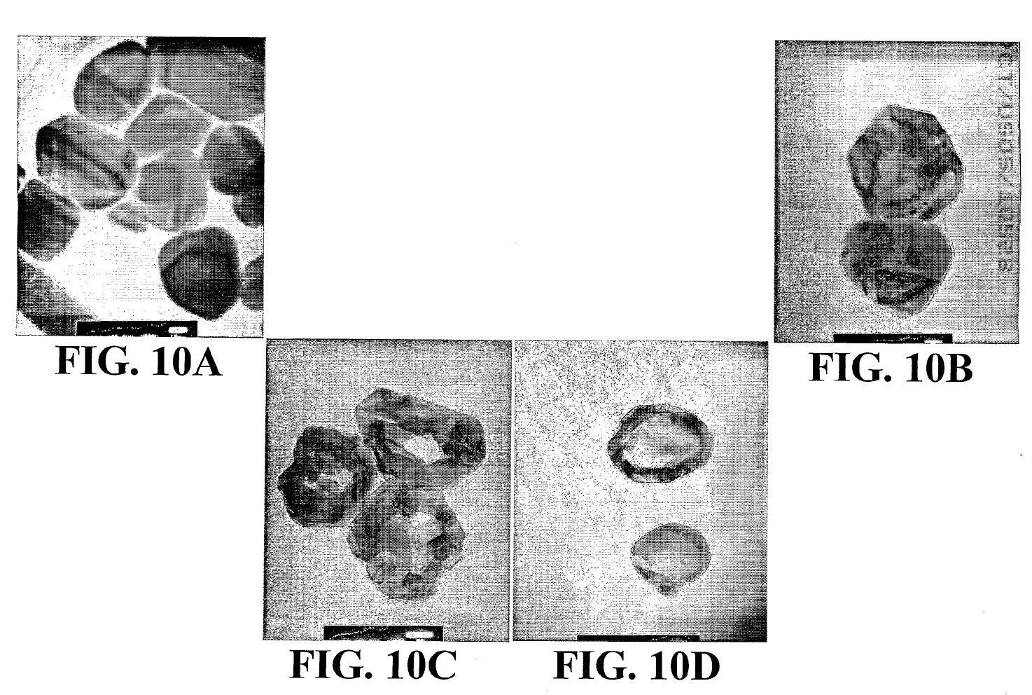

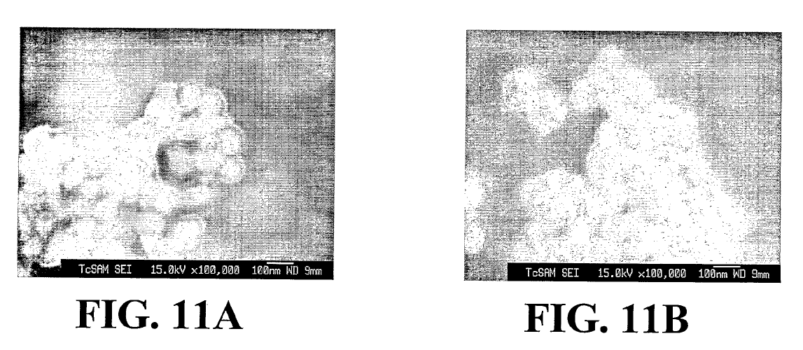

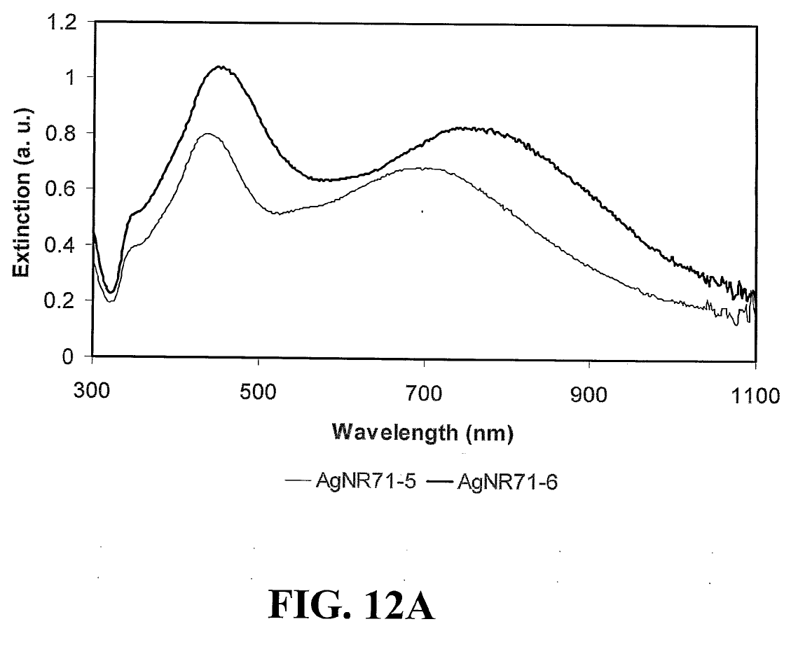

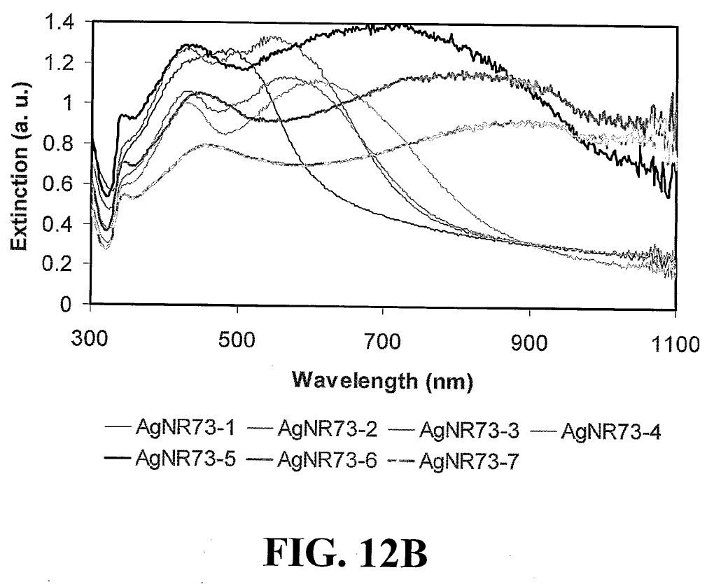

Referring now to FIGS. 9A-C, UV-vis spectra of silver core-gold nano-shell nano-particles of various sizes and core and shell thicknesses are show. FIG. 9A shows 45 nm silver core nano-particles having formed thereon different thicknesses of a gold nano-shell. FIG. 9B shows 55 nm silver core nano-particles having different thicknesses of a gold nano-shell. FIG. 9C shows 75 rm silver core nano-particles having different thicknesses of a gold nano-shell. The spectra clearly show that the metal-on-metal nano-shell nano-particles have considerable plasmon resonances in the infrared. Referring now to FIG. 10, TEM images of silver core-gold nano-shell nano-particles are shown. Referring now to FIG. 11, FE-SEM images of silver core-gold nano-shell nano-particles are shown. Referring now to FIGS. 12A&B, UV-vis spectra of silica core-silver nano-rod nano-particles are shown. These metal-on-metal nano-shell nano-particles can be prepared with considerable plasmon resonances in several different regions of the electro-magnetic spectra including the near infrared region.

### EXAMPLE 4

### Silica Core Nano-Particles Having Silver Nano-Rods

The present invention relates to metal oxide core nano-particles having formed on the surface of nano-rods of noble metals, where the nano-rods are grown from the surface assuming a variety of different direction and orientations on the surface. The nano-ceramic-core-noble-metal-nano-rod particles have optical properties ideally suited for electrooptical devices, drug-delivery, and cell targeting to thermally kill cells at targeted body sites.

Materials

The sodium hydroxide, ammonium hydroxide (30% NH₃), trisodium citrate dihydrate, nitric acid, borohydride, hydrochloric acid from EM Science, 3-aminopropyltrimethoxysilane (APTMS), tetraethylorthosilicate (TEOS) from Aldrich, ethanol from McKormick Distilling Co., silver nitrate from Mallinckrodt, cetyltrimethyl ammonium bromide (CTAB, 99+%) from Acros, and ascorbic acid from Chemalog were purchased from indicated companies. All the chemicals were used as received without purification. Water used in all reaction was purified to a resistance of 18 MΩ (Academic Milli-Q Water System; Millipore Corporation) and filtered using 0.22 μm filter membrane. All glassware used in the experiment were cleaned in an aquaregia solution first and cleaned in base bath prior to use.

Preparation of Amine-Functionalized Silica Nano-Particles

This is a modification of the well-known Stober method for making large silica nano-particles. (Stöber, W.; Fink, A.; Bohn, E. J. Colloid Interface Sci. 1968, 26, 62) 26.8 mL of ammonium hydroxide was added to 200 mL of absolute ethanol in a 500 mL two-necked round bottom flask and was stirred for 30 min at 30° C. Tetraethylorthosilicate (TEOS) 6 mL was quickly added into the mixture at 30° C. The color change of the mixture from colorless to milky white was observed in about 30 minutes and kept stirring it for overnight. 0.5 mL of excess APTMS was then added to the solution. The mixture was vigorously stirred for another 6-8 hours and heated to 85° C. for 1 hour to enhance covalent bonding of APTMS onto the silica nano-particles. (Waddell, T. G.; Leyden, D. E.; DeBello, M. T. J. Am. Chem. Soc. 1981, 103, 5303. van Blaaderen, A.; Vrij, A. J. J. Colloid Interface Sci. 1993, 156, 1) The amine-functionalized silica nano-particles were centrifuged RC-3B Refrigerated Centrifuige (Sorvall Instruments) at 2500 rpm (revolution per minute) for 1 hour and redispersed in 200 mL of ethanol twice. FE-SEM and TEM results showed no major differences between the unfunctionalized silica nano-particles and functionalized silica nano-particles from our experiment.

Preparation of Silver Seeds Attached to Silica Nano-Particles

Silver seed (˜3-4 nm in diameter) solution was prepared by an adaptation of the Nikhil et al. method to attach onto silica nano-particles. [Nikhil R. Jana, Latha Gearheart and Catherine J. Murphy Chem. Commun. 2001, 617. Nikhil R. Jana, Latha Gearheart, Catherine J. Murphy Adv. Mater. 2001, 13, 1389.] A 100 mL aqueous solution containing each 0.25 mM AgNO₃ and trisodium citrate was stirred for 5 minutes and 2.4 mL of a 0.01 M borohydride solution was quickly added into the mixture. The color of solution changed from colorless to bright yellow in few seconds which indicates the formation of ˜4 nm silver nano-particles. After 1 hour later, the seed solution was mixed with 2 mL of amine-functionalized silica nano-particles and stood for 2 hr at room temperature to make silver seed attached silica nano-particles by self-assembly. The mixture was then centrifuged at 3000 rpm for 1 hour, and the dark black-colored precipitate was re-dispersed in 100 mL of water. The mixture was sonicated for 5 minutes then centrifuged again for 30 minutes. The solution showed very light yellow color after the precipitate was re-dispersed in 100 mL of water.

Preparation of Silver Nano-Rods Grown on Silica Nano-Particles

First, 0.5 mL of 10 mM AgNO₃ solution was mixed with 20 mL of 80 mM CTAB and mix them carefully. The 1 mL of 100 mM ascorbic acid and varying amount of silver seed attached silica nano-particles (0.125˜2 mL) were added to the mixture and gently stirred it for 5 minutes. 0.2 mL of 1 M NaOH was added at the last step and gently shaken for another 5 minutes. The solution showed color change in 10 minutes dependant on the amount of silver seed attached silica nano-particles which that can control the size of silver nano rod onto silica surfaces. The final solution was centrifuge at 3000 rpm for 30 min to separate unreacted silver seed or free silver nano rod from the mixture. The precipitate was re-dispersed in 10 mL of water and sonicated for 5 minutes and centrifuged again for 30 minutes. The solution showed yellow, red, brown, blue, or green colors dependant on the size of silver rods after the precipitate was re-dispersed in 100 mL of water.

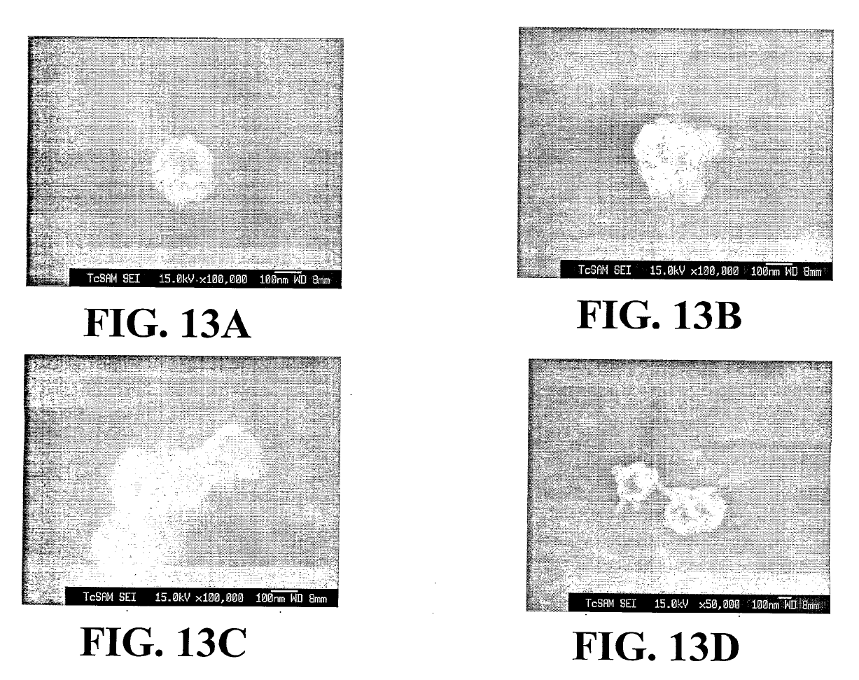

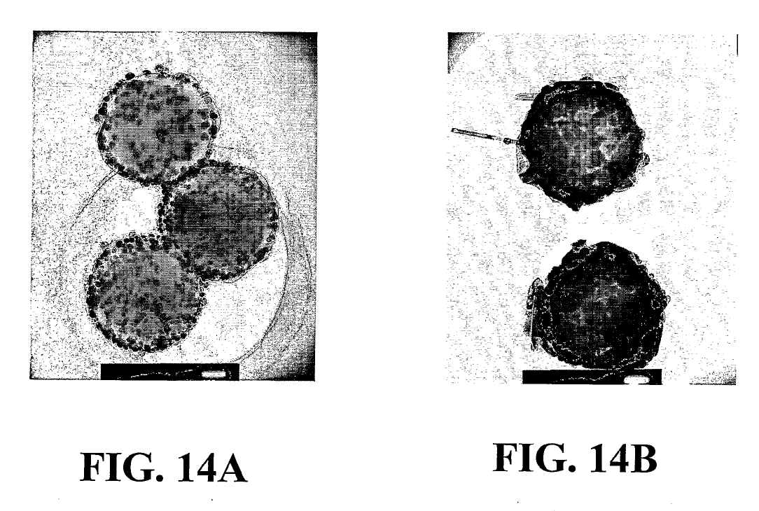

Referring now to FIGS. 13A&B, FE-SEM images of silica core-silver nano-rod nano-particles are shown from to different silica core solution concentrations, while FIGS. 14A-B depict TEM images of silica core-silver nano-rod nano-particles from two other preparations.

### EXAMPLE 5

### Synthesis of Hydrogel-Coated Gold Nano-Particles

The present invention relates to a targeted drug-delivery or absorbing system including metal or alloy nano-particles having deposited or grown thereon a hydrogel coating. The present invention also relates to hydrogel-coated nano-particles impregnated with one or more pharmaceuticals or bioactive agents. The present invention also relates to a method for treating body sites by locating the impregnated hydrogel-coated nano-shell nano-particles and irradiating the nano-particles to release the pharmaceuticals or bioactive agents.

Materials

The monomer N-isopropylacrylamide (NIPAM) was obtained from Acros (99%), recrystallized in hexane, and dried under vacuum before use. N,N′-methylenebisacrylamide (BIS, Acros), Acrylic acid (AAc, Acros, 99.5%), potassium hydroxide (KOH, EM, 85%), nitric acid (HNO₃, EM, 70%), ammonium persulfate (APS, EM, 98%), and oleic acid (OA, J. T. Baker) were all used as received from the indicated suppliers. Water used in all reactions, solution preparations, and polymer isolations was purified to a resistance of 10 MΩ (Milli-Q Reagent Water System, 3 Millipore Corporation) and filtered through a 0.2 μm filter to remove any particulate matter. In the preparation of gold nano-particles, trisodium citrate (EM, 99%) and hydrogen tetrachloroaurate (Strem, Au 99.9%) were used without purification.

Preparation of Gold Nano-Particles

Gold nano-particles were prepared via the common technique ofcitrate reduction, which has been described in detail. (Frens, G. Nature Phy. Sci. 1973, 241, 20. Turkevich, J.; Stevenson, P. C.; Hillier, J. Discussions Fara. Soc. 1951, 58, 55. Goodman, S. L.; Hodges, G. H.; Trejdosiewicz, L. K.; Linvinton, D. C. J. of Microscopy 1981, 123, 201.) The sizes of our gold nano-particles were always between 55 and 65 nm as judged by dynamic light scattering (DLS) and field emission scanning electron microscopy (FE-SEM). The glassware was cleaned first with strong acid (3/1 HC1/HNO₃) and then with strong base (saturated KOH in isopropyl alcohol) before use.

Synthesis of Hydrogel-Coated Gold Nano-Particles

(Quanroni, L.; Chumanov, G. J. Am. Chem. Soc. 1999, 121, 10642. Clark, H. A.; Campagnok, P. J.; Wuskell, J. P.; Lewis, A.; Loew, L. M. J. Am. Chem. Soc. 2000, 122, 10234.) The hydrogel-coated gold nano-particles were prepared by surfactant-free emulsion polymerization (SFEP) in aqueous solution. In a three-necked round-bottomed flask equipped with a reflux condenser and an inlet for argon gas, gold colloidal solutions were diluted with purified Milli-Q water to give a maximum of ˜0.25 a.u. at 530 nm. The solution was purged with argon for 1 h and was bubbled through the solution for the duration of the reaction to remove any oxygen, which can intercept radicals and disrupt the polymerization. The solution was agitated using a football-shaped Teflon-coated magnetic stirring bar. Degassed oleic acid 1.6 mL (0.001 M), which has a low affinity toward gold, was then added to the stock solution under argon. The mixture was stirred for 1 h and placed in an ultra-sonicator for 15 minutes. An approximately 94:6 wt % ratio of NIPAM 26.1 mL (0.01 M):AAc 1.6 mL (0.01 M) and 2 mL of BIS (0.01 M) was then added and stirred for 15 minutes to give homogeneity. The solution was then heated to 71° C. in an oil bath, and then APS 0.8 mL (0.01 M) was added to initiate the polymerization. The reaction time, which depended on the amount of starting materials, was varied between 6 and 8 h. At the end of this period, the solution was cooled and filtered through a 1 μm membrane to remove any micron-sized impurities and/or any aggregated particles. The filtered solution was centrifuged at 20° C. for 2 h at 3500 rpm with RC-3B Refrigerated Centrifuge (Sorvall Instruments), and the supernatant was separated to remove unreacted materials, soluble side products, and seeds of pure polymer. The purified nano-particles were then diluted with pure Milli-Q water and stored at room temperature for later use. The size of the hydrogel-coated gold particles was varied between 100 and 230 nm by controlling the amount of monomer and initiator as well as the reaction time.

Characterization of Gold and Hydrogel-Coated Gold Nano-Particles

To characterize the pure gold nano-particles and hydrogel-coated gold nano-particles, we used field emission scanning electron microscopy (FE-SEM), energy dispersive X-ray (EDX) analysis, ultraviolet-visible (UV-vis) spectroscopy, and dynamic light scattering (DLS). Due to our interest in thick hydrogel coatings, our most thorough analyses were focused on the hydrogel-coated gold nano-particles having ˜230 nm diameters.

We employed a Cary 50 Scan UV-vis optical spectrometer (Varian) with Cary Win UV software to characterize the optical properties of the bare gold nano-particles and the hydrogel-coated gold nanoparticles. UV-vis spectra of the prepared gold nano-particles were collected by diluting the particles with Milli-Q water, transferring them to an optical glass cell, and scanning over a range ofwavelengths (400-1100 nm). The hydrogel-coated gold nano-particles were analyzed as prepared (i.e., without dilution). For consistency, UV-vis spectra of the distinct batches of nano-particles were collected both before and after coating with the hydrogel.

Analysis by FE-SEM was performed using a JSM 6330F (JEOL) instrument operating at 15 kV and equipped with a setup for elemental analysis by EDX (Link ISIS software series 300, Oxford Instruments). To collect both FE-SEM images and EDX data, the gold nano-particles and hydrogel-coated nano-particles were deposited on Formvar-coated copper grids and completely dried at room temperature overnight prior to analysis. The samples were then coated with a carbon film (2.5 nm thick) using a vacuum sputterer. The gold and hydrogel-coated gold nano-particles were examined by FE-SEM (magnification 20,000-100,000X) to demonstrate the overall morphological uniformity of the nano-particles and by EDX to confirm the presence of the gold nano-particle core.

For the DLS measurements, an ALV-5000 Multiple Tau Digital Correlation instrument operating at a light source wavelength of 514.5 nm and a fixed scattering angle of 90° C. was used to measure particle size as a function of temperature and pH for gold and hydrogel-coated gold nano-particles. The samples were measured at dilute concentrations with precise control over the temperature (especially at higher temperatures to reduce artifacts resulting from convection currents in the samples). For all samples, data were collected from 20-60° C. All data showed good Gaussian distribution curves, and the standard deviation of the distribution was 5 to 20% of the mean for all samples.

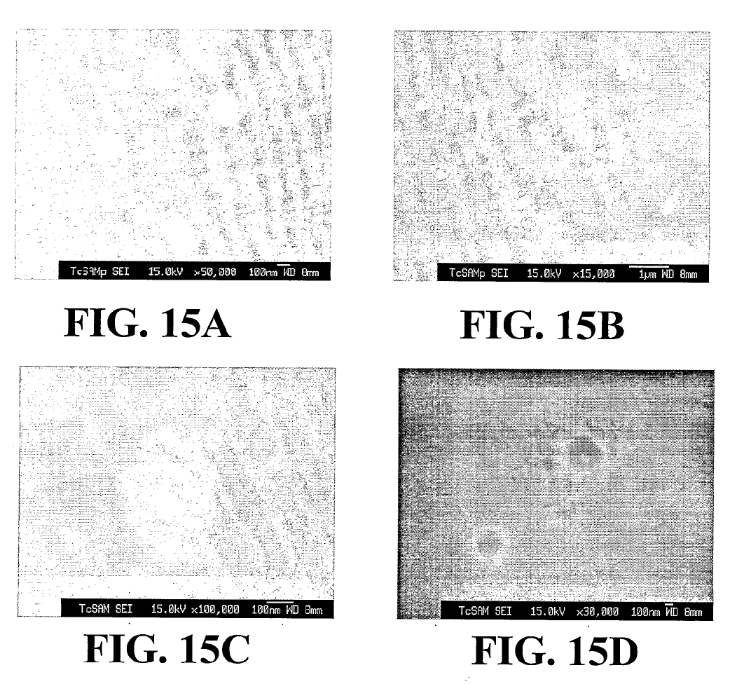

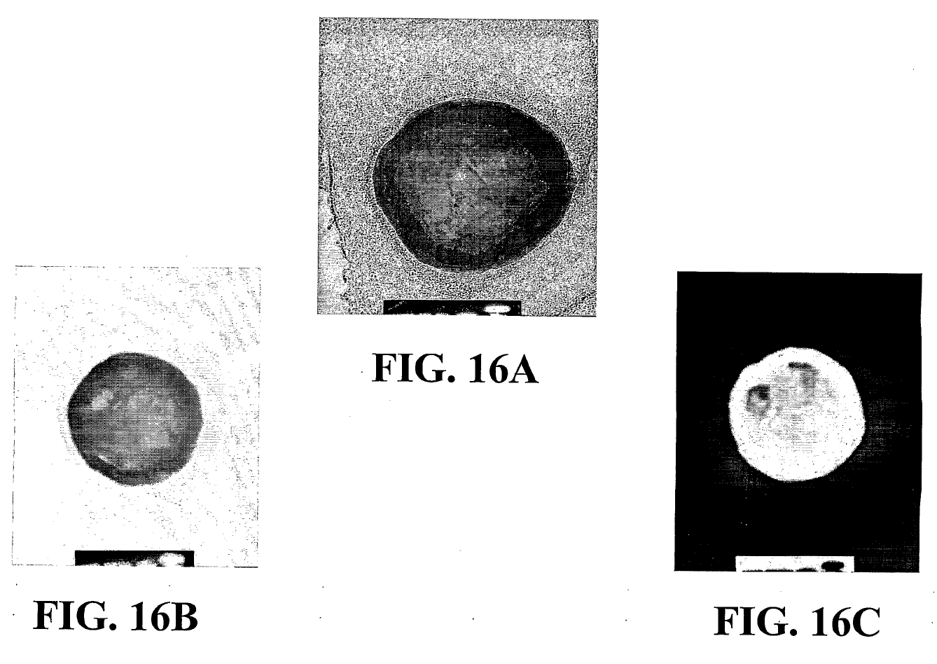

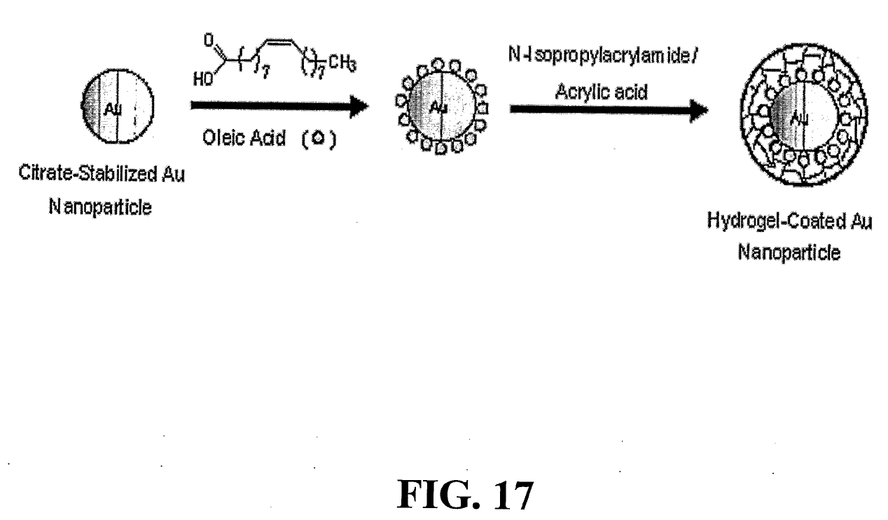

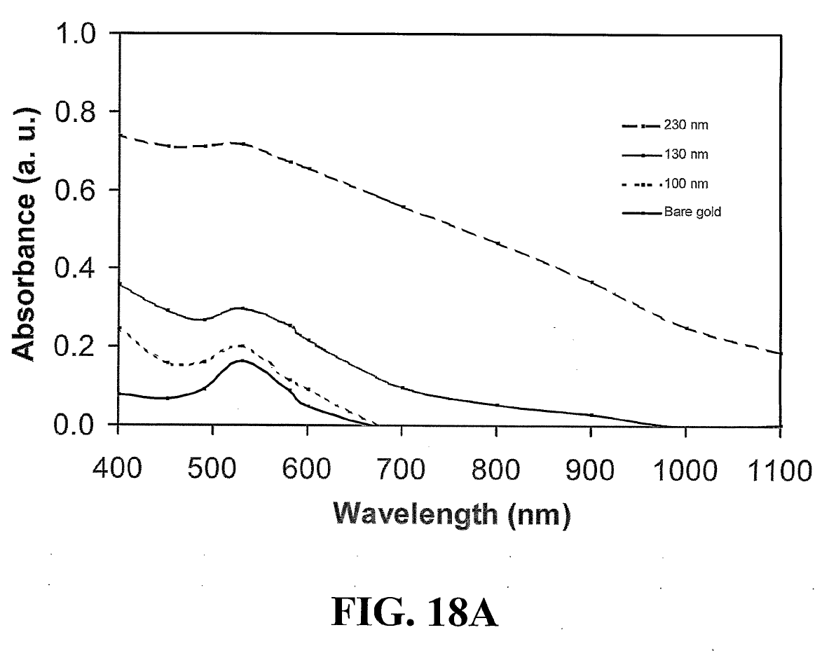

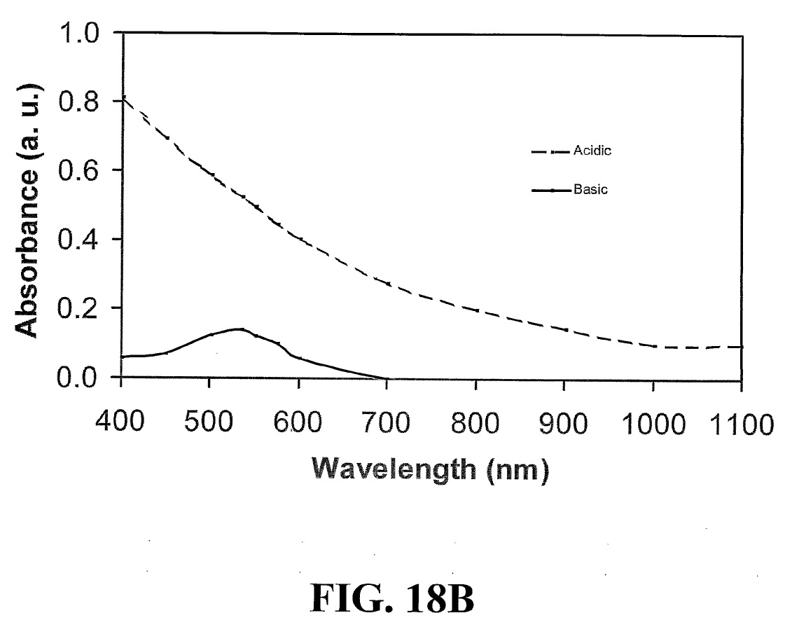

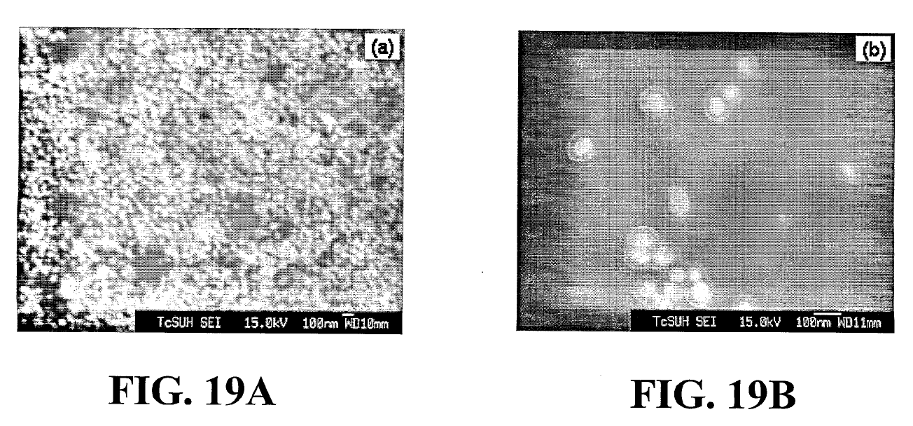

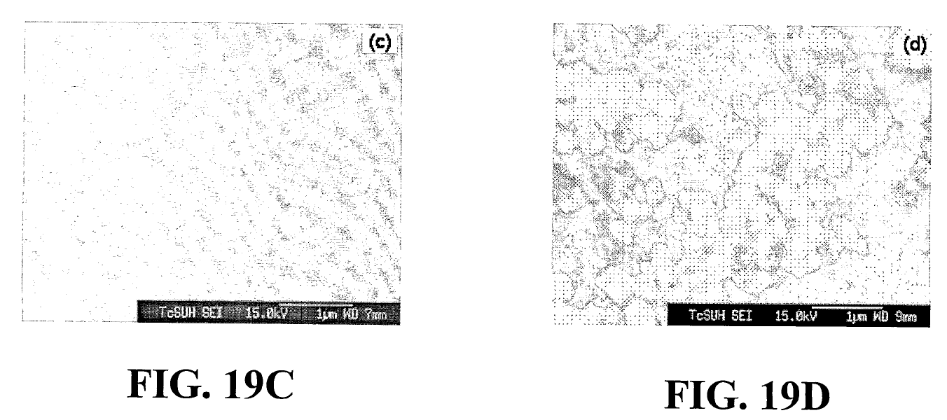

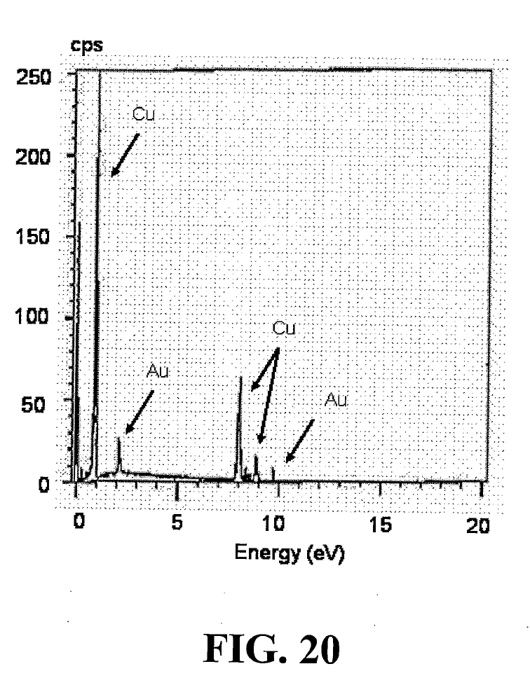

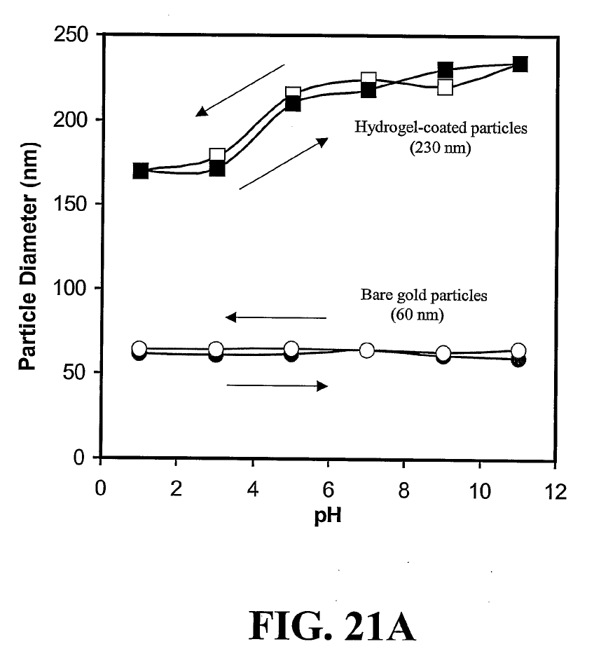

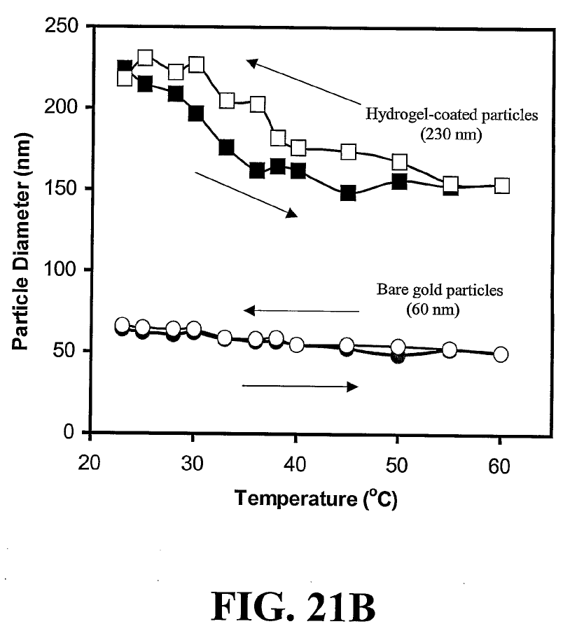

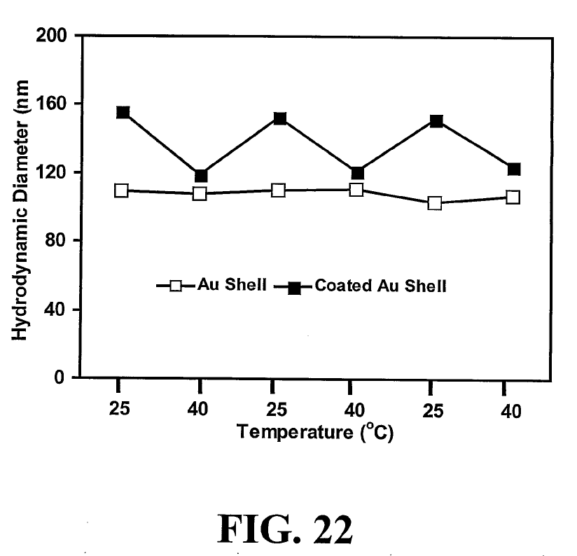

Referring now to FIGS. 15A-B, FE-SEM images of discrete hydrogel-coated gold nano-particles are shown. In FIG. 15A, discrete hydrogel-coated gold nano-particles having a 120 nm core diameter are shown, while in FIG. 15B discrete hydrogel-coated gold nano-shell nano-particles having a 100 nm core diameter are shown. Referring now to FIG. 16A-B, TEM images of discrete hydrogel-coated gold particles are shown. In FIG. 16A discrete hydrogel-coated gold nano-particles having a 120 nm core diameter are shown, while in FIG. 16B discrete hydrogel-coated gold nano-particles a 100 nm core diameter are shown. Referring now to FIG. 17, a schematic of a preferred discrete hydrogel coating process is shown where a gold nano-particles is first seeded with polymers nodes and then the hydrogel is grown from the nodes. Referring now to FIGS. 18A-B, absorbance spectra of hydrogel-coated gold nano-particles in neutral (FIG. 18A) and acidic or basic media (FIG. 18B) are shown. Referring now to FIGS. 19A-D, FE-SEM images of ˜60 nm bare gold nano-particles (FIG. 19A), ˜100 nm hydrogel-coated gold nano-particles (FIG. 19B), ˜130 nm hydrogel-coated gold nano-particles (FIG. 19C), and ˜230 hydrogel-coated gold nm nano-particles (FIG. 19D). Referring now to FIG. 20, an EDX spectrum of hydrogel-coated gold nano-particles is shown clearly evidencing the gold lines. Referring now to FIG. 21A, a plot of particle size verse pH for bare gold nano-particles and hydrogel-coated gold nano-particles is shown, where the gold nano-particles do not undergo a change in size; while the hydrogel-coated gold nano-particles undergo an increase in size between pH 2 and 4. Referring now to FIG. 21B, a plot of particle size verses temperature for bare gold nano-particles and hydrogel-coated gold nano-particles is shown, where the gold nano-particles do not undergo a change in size; while the hydrogel-coated gold nano-particles undergo a decrease in size starting at about 30° C. Referring now to FIG. 22, a plot of hydrodynamic diameter (nm) verses temperature, where the temperature is cycled between about 25° C. and about 40° C. due to periodic irradiation of light within the plasmon resonance spectral band.

### EXAMPLE 6

### Synthesis of Discrete Hydrogel-Coated Gold Shell Nano-Particles

The present invention relates to a targeted drug-delivery or absorbing system including nano-shell nano-particles having deposited or grown thereon a hydrogel coating. The present invention also relates to hydrogel-coated nano-shell nano-particles impregnated with one or more pharmaceuticals or bioactive agents. The present invention also relates to a method for treating body sites by locating the impregnated hydrogel-coated nano-shell nano-particles and irradiating the nanoparticles to release the pharmaceuticals or bioactive agents.

Materials

The N-isopropylacrylamide (NIPAM) monomer was obtained from Acros (99%), recrystallized in hexane, and dried at room temperature before use. Other chemicals were used: acrylic acid (AAc), N,N′-methylenebisacrylamide (BIS) from Acros and nitric acid, hydrochloric acid, ammonium persulfate (APS), sodium hydroxide, ammonium hydroxide (30% NH₃), formaldehyde, potassium carbonate, trisodium citrate from EM Science, terakis(hydroxoymethyl)phosphonium chloride (THPC), 3-aminopropyltrimethoxysilane tetraethylorthosilicate (APTMS) from Aldrich, hydrogen tetrachloroaurate (Au 99.9%) from Strem, and ethanol from McKormick Distilling Co., oleic acid from J. T. Baker. Water used in all reactions, solution purifications, and polymer isolations was used as a resistance of 18 MΩ (Academic Milli-Q Water System, Millipore Corporation) and filtered through a 0.22 μm filter. All glassware used in the experiment were cleaned in an aquaregia solution first then cleaned in base bath and rinsed in Mill-Q water prior to use.

Characterization Methods

All the particles were analyzed by ultraviolet-visible (UV-vis) spectroscopy, field emission scanning electron microscopy (FE-SEM), dynamic light scattering (DLS), transmission electron microscopy (TEM) and energy dispersive X-ray (EDX).

JSM 6330F (JEOL) FE-SEM instrument was used to observe the morphologyofthe particles operating at 15 kV. In order to get high resolution images, all samples were placed on the carbon-coated copper grid and completely dried at room temperature overnight prior to the carbon coating. The samples were then coated with carbon films using sputtering equipment to improve electrical conductivity. The samples were examined by FE-SEM images (magnification 20,000-150,000X) to show the overall uniformity and morphology of nano-particles on the surface.

A JEM-2000 FX electron microscope (JEOL) TEM analysis was accomplished at accelerating voltage 200 kV. All the TEM samples were deposited on 300 mesh Holey carbon coated copper grids and dried before they were examined.

The UV-vis spectra were obtained by using a Cary 50 Scan UV-visible spectroscopy over the range from 300-1100 nm wavelength. All samples were centrifuged and re-dispersed in Milli-Q water and transferred into a UV cell with optical glass windows.

Preparation of Amine-Functionalized Silica Nano-Particles

This is a modification of well-known Stober method for making large silica nano-particles. (Stöber, W.; Fink, A.; Bohn, E. J. Colloid Interface Sci. 1968, 26, 62.) 26.8 mL of ammonium hydroxide was added to 200 mL of absolute ethanol in a 500 mL two-necked round bottom flask and stirred for 30 minutes at 30° C. Six (6) mL of TEOS was quickly added into the mixture, and the silica particle formation was observed by the color changes of the solution from colorless to milky white within 30 minutes. The mixture was kept stirring for 24 hour and 0.5 mL of excess APTMS was then added to the solution. The mixture was vigorously stirred for another 6-8 hours and heated to 85° C. for 1 hour to enhance covalent bonding of APTMS onto the silica particles. (Waddell, T. G.; Leyden, D. E.; DeBello, M. T. J. Am. Chem. Soc. 1981, 103, 5303. van Blaaderen, A.; Vrij, A. J. J. Colloid Interface Sci. 1993, 156, 1.) The solution was centrifuged at 2500 rpm (revolution per minute) for 1 h using RC-3B Refrigerated Centrifuge (Sorvall Instruments) and re-dispersed in 200 mL of ethanol twice. FE-SEM and TEM results showed no major differences between the unfunctionalized silica particles and fuictionalized silica particles from our experiments (data now shown).

Preparation THPC Gold Seed and K-Gold Solution

The THPC gold seed solution was made using a modification of the Duff et al method. (Duff, D. G.; Baiker, A. Langmuir 1993, 9, 2301. Duff, D. G.; Baiker, A. Langmuir 1993, 9, 2310. Teo, B. K.; Keating, K.; Kao, Y-H. J. Am. Chem. Soc. 1987, 109, 3494.) To make ˜2-4 nm THPC gold seed nanoparticles, 1 mL of 1 M NaOH, 2 mL of THPC (12 μL of 80% THPC in 1 mL of water), and 200 mL of Milli-Q water were mixed in a 250 mL flask and vigorously stirred at least 15 minutes. 4 mL of 1% aqueous HAuCl₄3H₂O were added quickly to the solution and stirred about 30 more minutes. The color of the solution changed very quickly from colorless to dark red. The size of gold nano-particles could be varied, but our nano-particles possessed diameters of in a range between about 2 and about 3 nm. This solution was then stored in the refrigerator for at least three days.

For the K-gold solution, 0.05 g potassium carbonate in 200 mL water was stirred for at least 15 minutes to ensure dissolving of K₂CO₃ and then 2 mL of 1% HAuCl₄3H₂O was added. The color of the solution changed from yellow to colorless within 40 minutes.

Formation of THPC Gold Nano-Particles Attached to Silica Nano-Particles

This is the modification of Westscott et. al. method to make gold seed attached onto silica core nano-particles. (Westscott, S. L.; Oldenburg, S. J.; Lee, T. R.; Halas, N. J. Langmuir 1998, 14, 5396.) The THPC gold nano-particles were deposited onto the silica particles by mixing THPC gold nano-particles and amine-functionalized silica nano-particles for overnight. About 1 mL of amine-functionalized silica particles dispersed in ethanol was placed into a 50 mL ofthree-day aged THPC gold nano-particles in a centrifuge tube. The mixture was shaken for a couple of minutes and left overnight to attach gold seeds to silica particles by self-assembly. The mixture was then centrifuged at 3000 rpm for 1 hour and the dark red-colored precipitate were re-dispersed in 50 mL of water. The mixture was briefly sonicated for 5 minutes and centrifuged again for additional 60 minutes. The solution showed very light red color after the precipitate was re-dispersed in 50 mL of water.

Gold Nano-Shell Growth

(T. Pham; J. B. Jackson; N. J. Halas; T. R. Lee Langmuir 2002, 18, 4915.) In order to make complete gold layer onto THPC gold seed attached silica nano-partciles, the prepared K-gold solution (4 mL) was placed in a 25 mL beaker and added different amount of THPC gold seed attached silica nano-particles (0.1 to 2 mL) to produce different thickness of gold shells. The mixture was stirred at least 5 minutes and added 0.01 mL of reducing agents such as formaldehyde and borohydride to reduce K-gold solution. The color change of solution took place from colorless to blue, green, and yellowish green dependent on the shell thickness. The gold nano-shells were centrifuiged and re-dispersed in Milli-Q water to remove unreacted free gold seed particles.

Preparation of Hydrogel-Coated Gold Nano-Shell Nano-Particles

The hydrogel-coated gold nano-shell nano-particles were prepared by modification of the method from Quanroni et. al. (Quanroni, L.; Chumanov, G. J. Am. Chem. Soc. 1999, 121, 10642.) The gold shell solution was diluted with 0.001M potassium carbonate solution to slow down the aggregation phenomenon and to adjust the concentration of solution, which has a absorption maximum of ˜0.6 a.u. at 800 nm using UV-vis spectroscopy.

The hydrogel-coated gold nano-shell nano-particles were prepared in a 500 mL three-necked round-bottomed flask equipped with a reflux condenser and filled with argon gas. Oleic acid (0.00174 mL; 3×10⁻⁵ mol) was added to the gold shell solution and stirred for 45 minutes and then placed in an ultrasonic bath for 15 minutes. An approximately 94:6 wt % ratio of NIPAM (0.125 g; 4.4∴10⁻³ mol): AAc (0.0075 g; 5×10⁻⁴ mol) and cross-linker BIS (0.003 g; 1×10⁻⁴ mol) were then added, and the mixture was stirred for 15 minutes. The solution was heated to 70° C. in an oil bath, and then air-free APS (0.0048 g; 1×10⁻⁴ mol) was quickly added to initiate the polymerization. The reaction was allowed to proceed for ˜16 hours, after that the solution was filtered through a 1 μm membraneto remove anymicron-sized impurities. The filtered solutionwas centrifuiged at 2500 rpm for 1 hour at 30° C. with an RC-3B Refrigerated Centrifuge (Sorvall Instruments). Afterward, the top layer containing unreacted materials or water-soluble side products was removed by decantation. The purified hydrogel-coated gold nano-shell particles were then diluted with Milli-Q water and stored at room temperature for subsequent analyses.

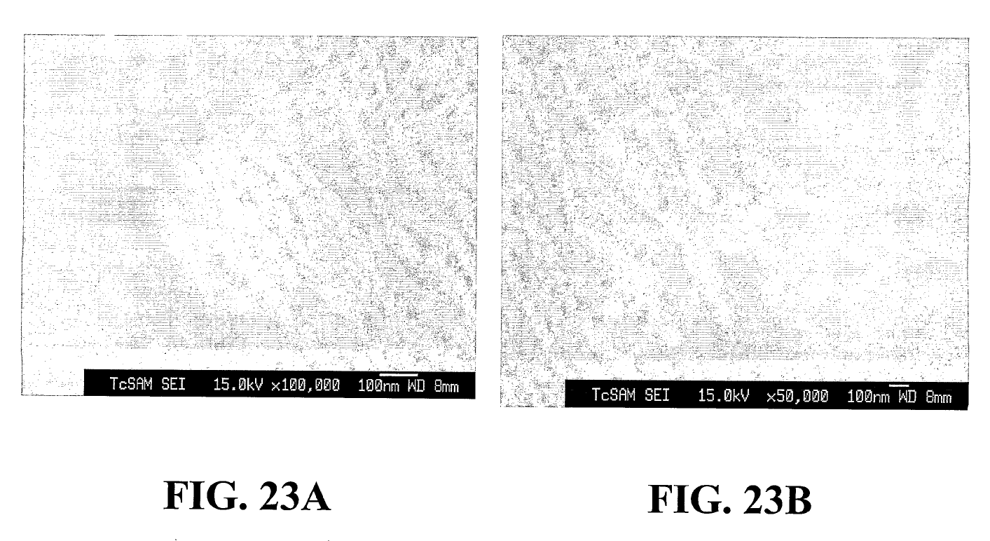

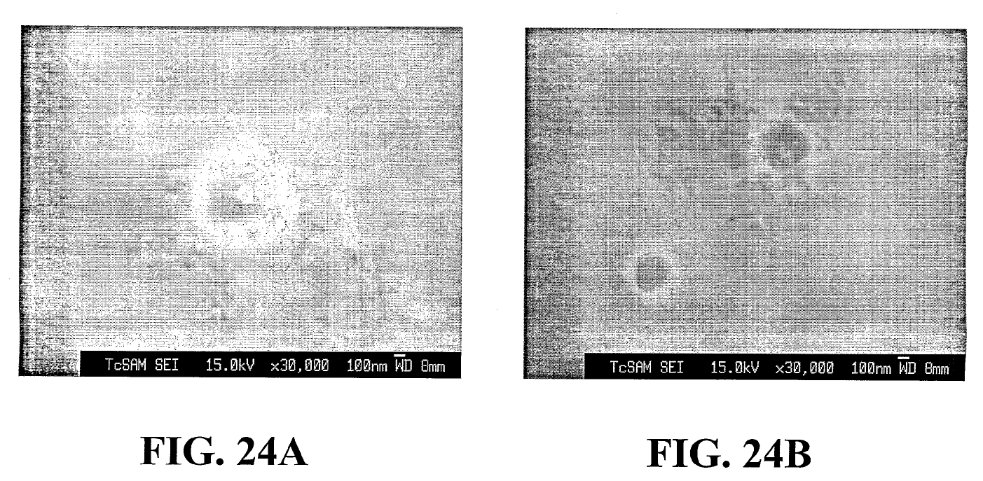

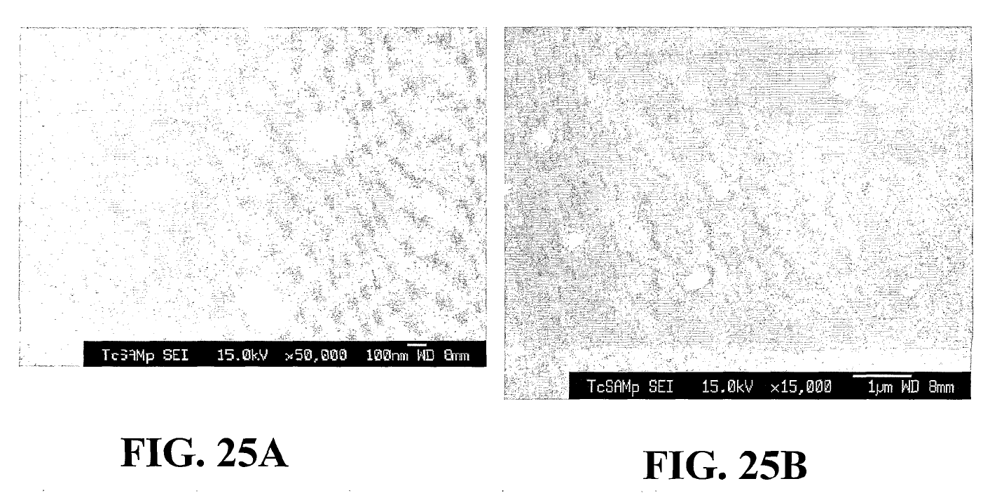

Referring now to FIGS. 23A&B, FE-SEM images of hydrogel-coated gold nano-shell nano-particles having a nano-shell nano-particles are ˜100 nm with a thin hydrogel coating are shown. Referring now to FIG. 24A&B, FE-SEM images of hydrogel-coated gold nano-shell nano-particles having a nano-shell nano-particles are ˜100 nm with a thick hydrogel coating are shown. Referring now to FIGS. 25A&B, FE-SEM images of hydrogel-coated gold nano-shell nano-particles having a nano-shell nano-particles are ˜120 nm with a thin hydrogel coating are shown.

### EXAMPLE 7

### Preparation of Metal Nano-particles Having Metal Nano-Shells Materials

Sodium citrate dihydrate, nitric acid, hydrochloric acid (EM Science), potassium carbonate (J. T. Baker), hydrogen teterachloroaurate-(III) hydrate (Strem), terakis(hydroxoymethyl)phosphonium chloride (THPC, Aldrich), silver nitrate (Mallinckrodt), and L-ascorbic acid from Chemalog were purchased from indicated companies. All the chemicals were used as received without purification. Water was purified to a resistance of 18 MΩ (Academic Milli-Q Water System; Millipore Corporation) and filtered using 0.22 μm membrane filter. All glassware used in the experiment were cleaned in a strong acid and base prior to use.

Preparation of Large Silver Nano-Particle Cores

This is a slight modification of well known Lee and Meisel method to make variable sizes of silver nanoparticles (over 40 nm in diameter). (Langmuir 2001, 17.574-577. Journal of Colloid and Interface and Science, 1983, 93, 545-555. J. Phys. Chem. 1982, 86,3991.) 200 mL of a 10⁻³ M AgNO₃ solution was heated to boiling, and added 4 ml of a 1% trisodium citrate as soon as it reaches boiling. The mixture was kept stirring and boiling for 45 min to get homogeneous silver nano-particles (˜60 nm). Other sizes of silver nano-particles (˜45 and ˜75 nm) were prepared from different concentrations of silver nitrate with constant amount of sodium citrate.

Preparation of Small Silver Nano-Particle Cores

Chen et al. method was adopted to make small silver nanoparticles with 10-15 nm in diameter. (J. Phys. Chem. 2002, 106,10777.) 0.6 mL of 10 mM NaBH4 was added into the stirring mixture containing 0.5 mL of 10 mM silver nitrate and 20 mL of 1.25 mM sodium citrate. The solution was stirred for 5 more min and aged for 2 hr before use.

Preparation of Gold Nano-Particle Cores

The variable sizes of gold nanoparticles were prepared via the common technique of citrate reduction, which has been described in detail elsewhere. (Frens, G. Nature Phy. Sci. 1973, 241, 20. Turkevich, J.; Stevenson, P. C.; Hillier, J. Discussions Fara. Soc. 1951, 58, 55. Goodman, S. L.; Hodges, G. H.; Trejdosiewicz, L. K.; Linvinton, D. C. J. of Microscopy 1981, 123, 201.)

K₂CO₃-Gold (K-Gold) Preparation

To make K-gold solution, 0.05 g ofpotassium carbonate (K₂CO₃) in 200 ml of Milli-Q water was stirred for at least 15 min to dissolve K₂CO₃ completely and added 4 mL of 1 wt % HAuCl₄.3H₂O. The color of solution changes from yellow to almost colorless within 30 min.

Gold-Coated Silver and Gold Nano-Particles

To grow the gold layers on the silver nano-partcle cores, 10 mL of one day old K-gold solution was placed in a 25 mL beaker with stir bar and added prepared silver nano-particle cores (1 to 9 mL) to produce different thickness of gold layers. The mixture was kept stirring at least 10 min and 0.6 ml of 100 mM L-ascorbic acid was added quickly. The color change occurred from light yellow to red, violet, and blue dependent on the thickness of the shells. The mixture was centrifuiged at 2500 rpm for 1 h using RC-3B Refrigerated Centrifuige (Sorvall Instruments) and redispersed in Milli-Q water for the analysis. The gold-coated gold nano-particles also achieved the same way as gold-coated silver nano-particles.

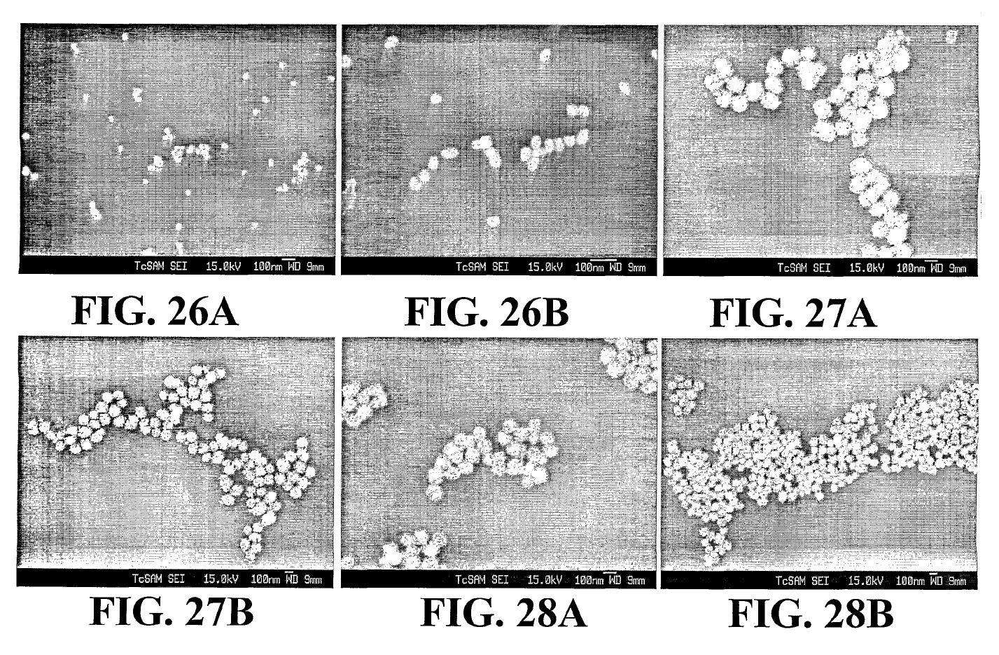

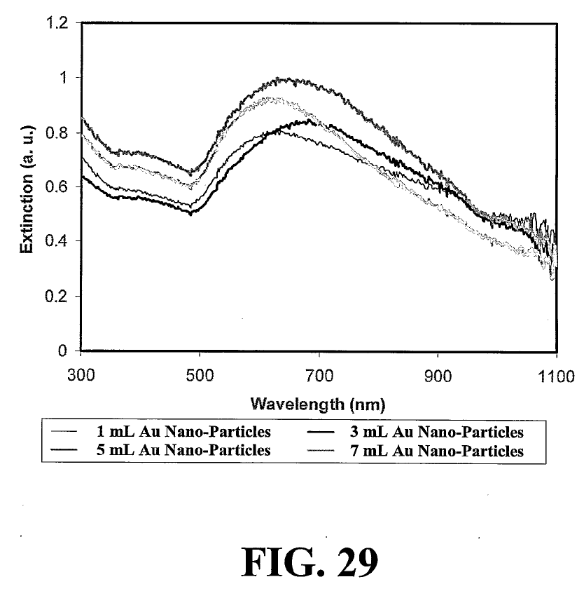

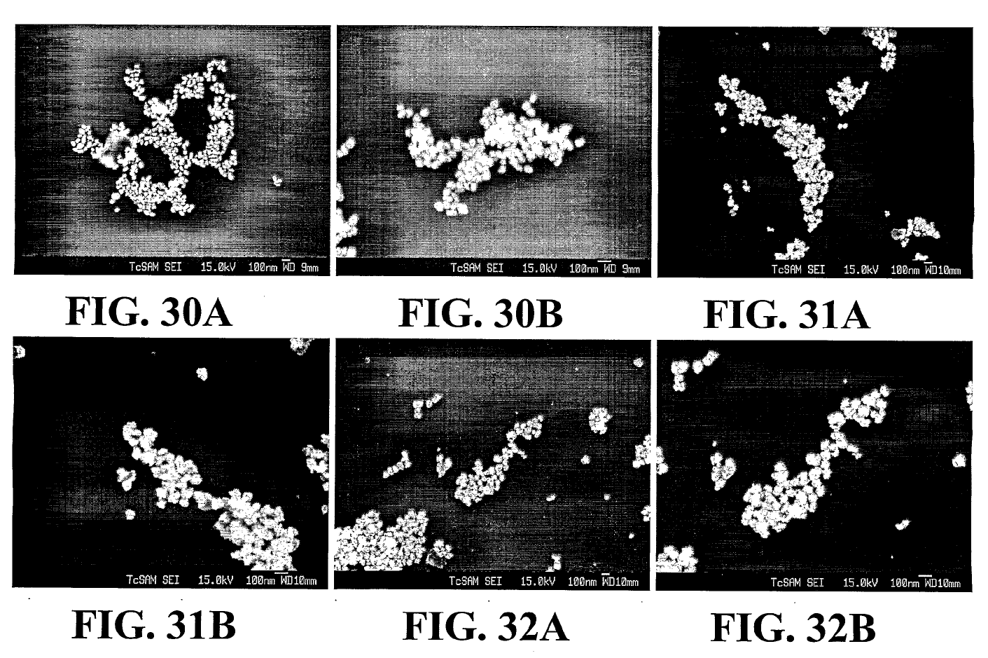

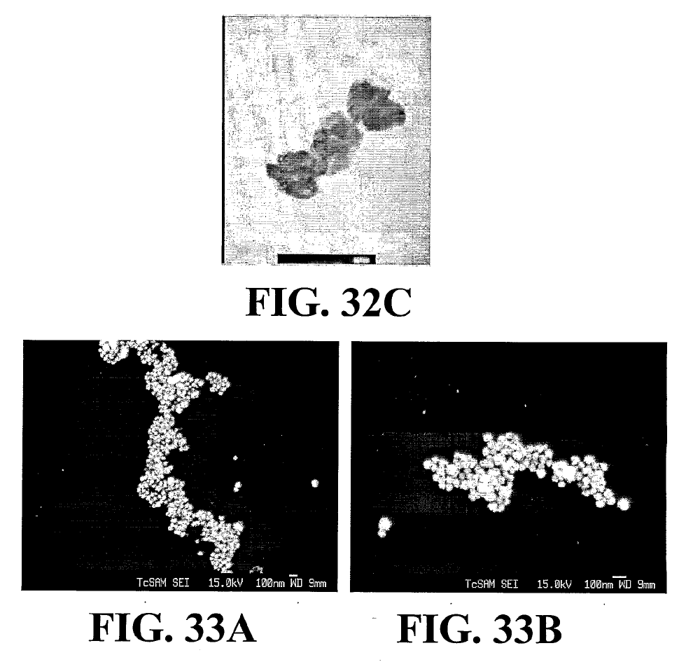

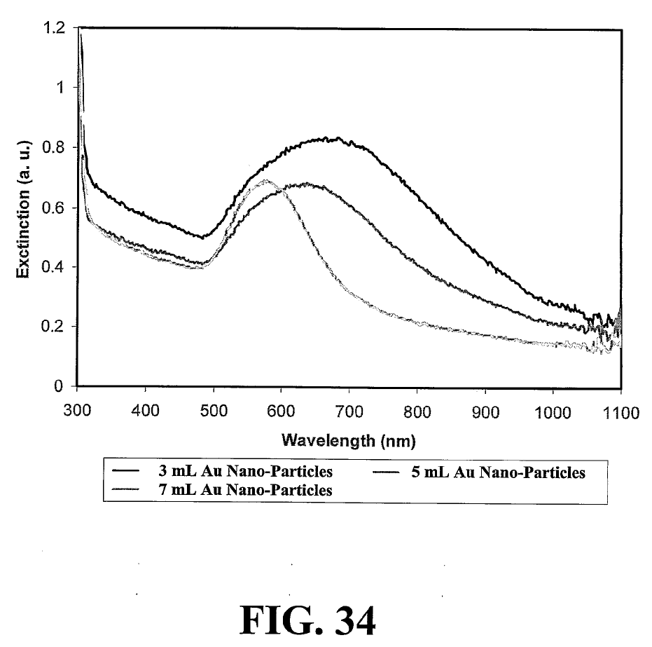

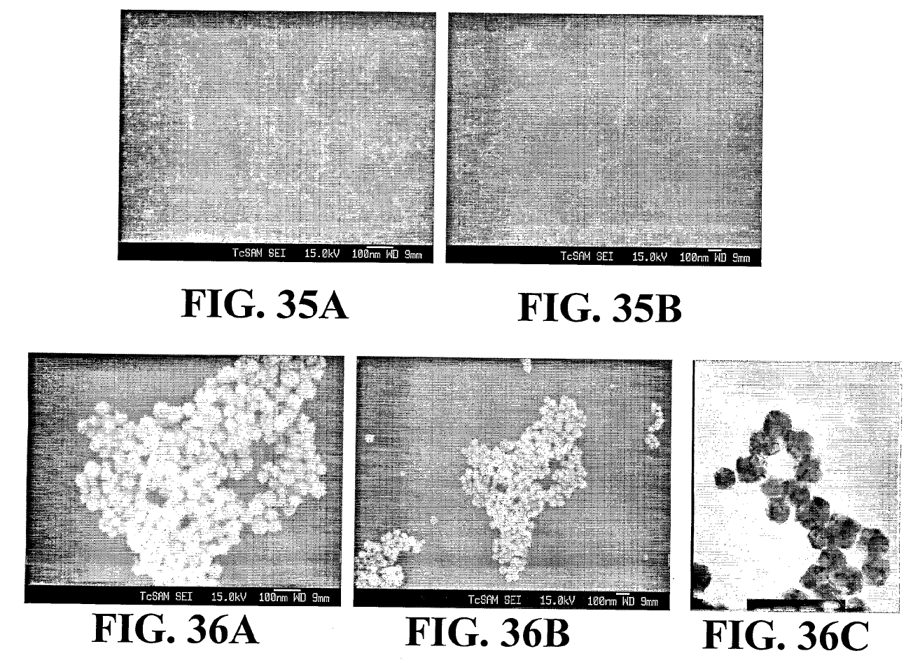

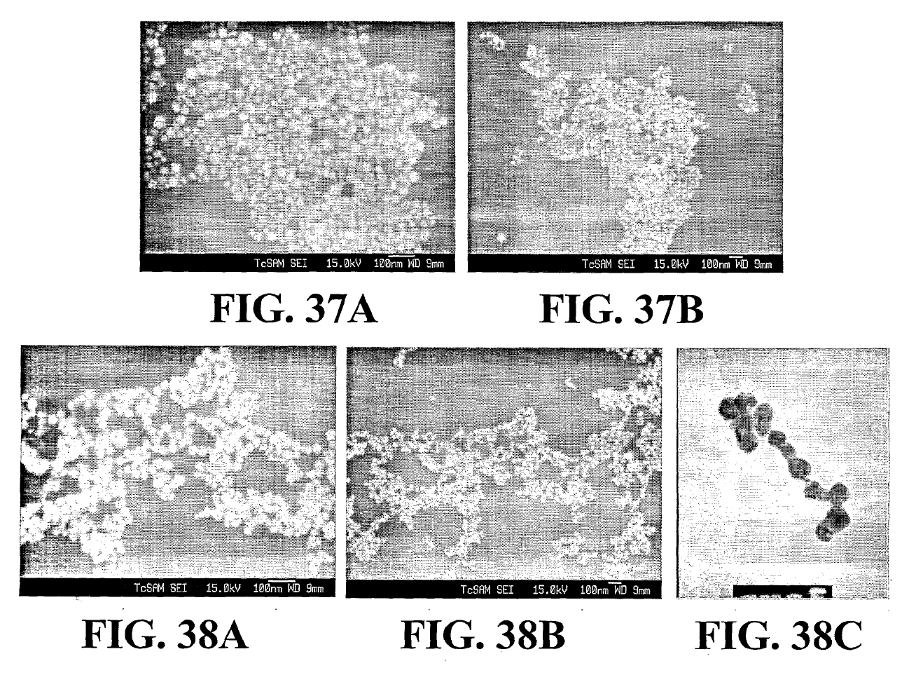

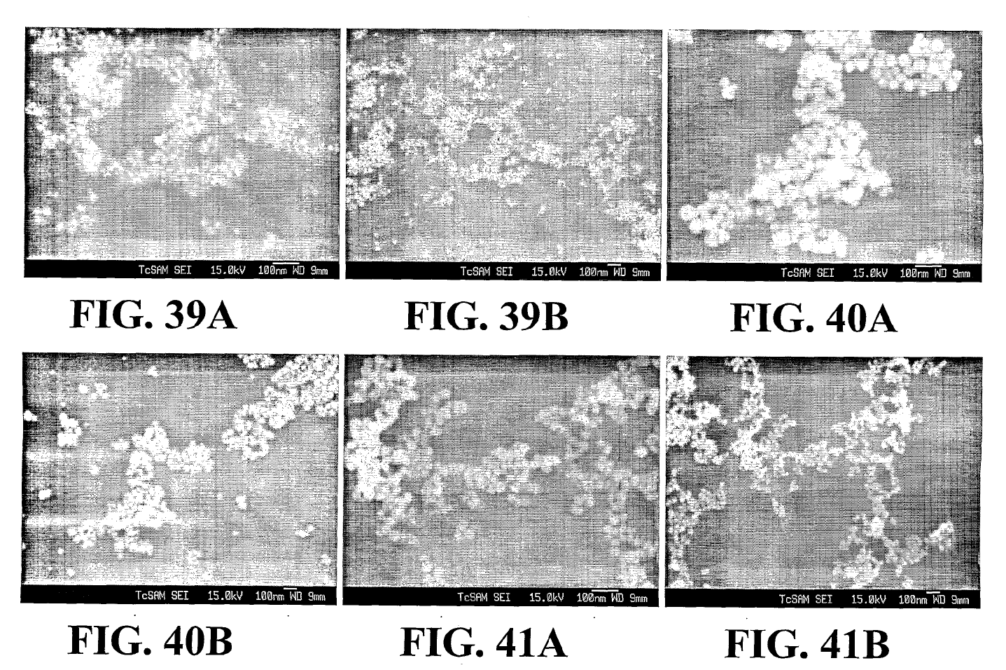

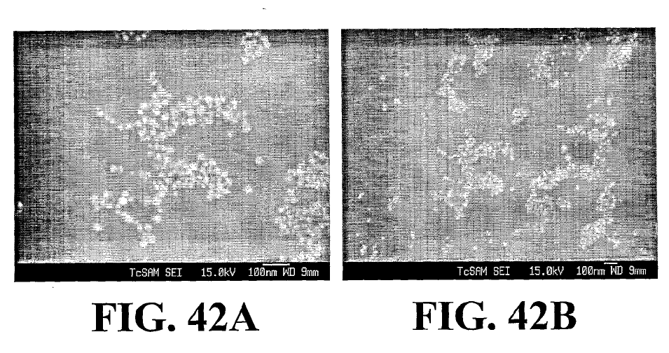

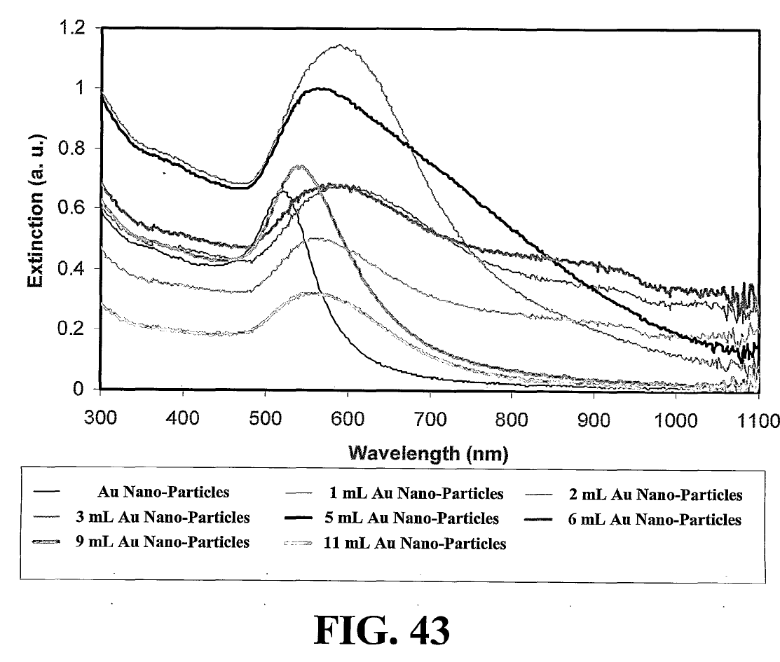

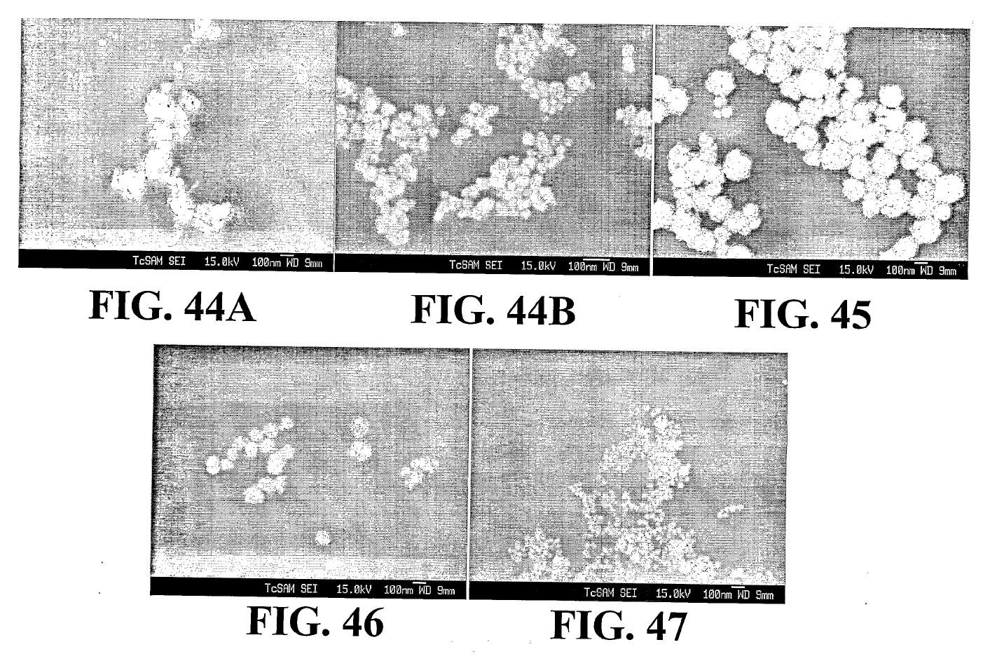

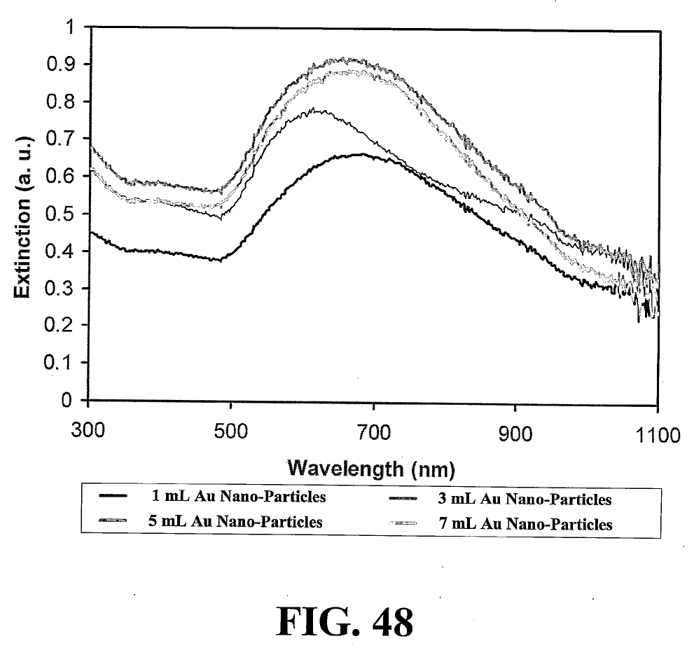

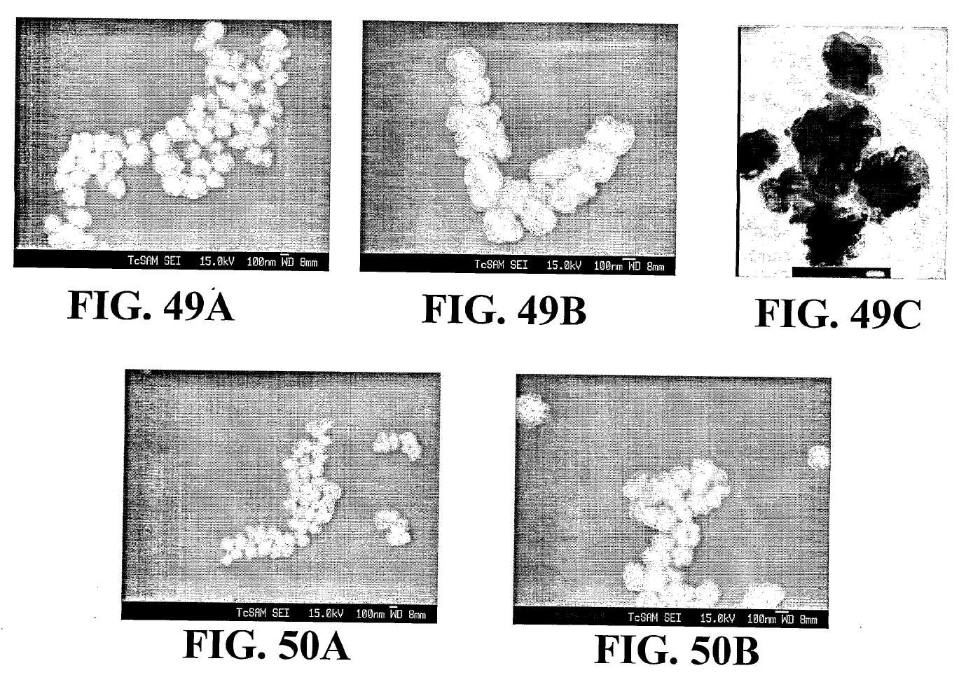

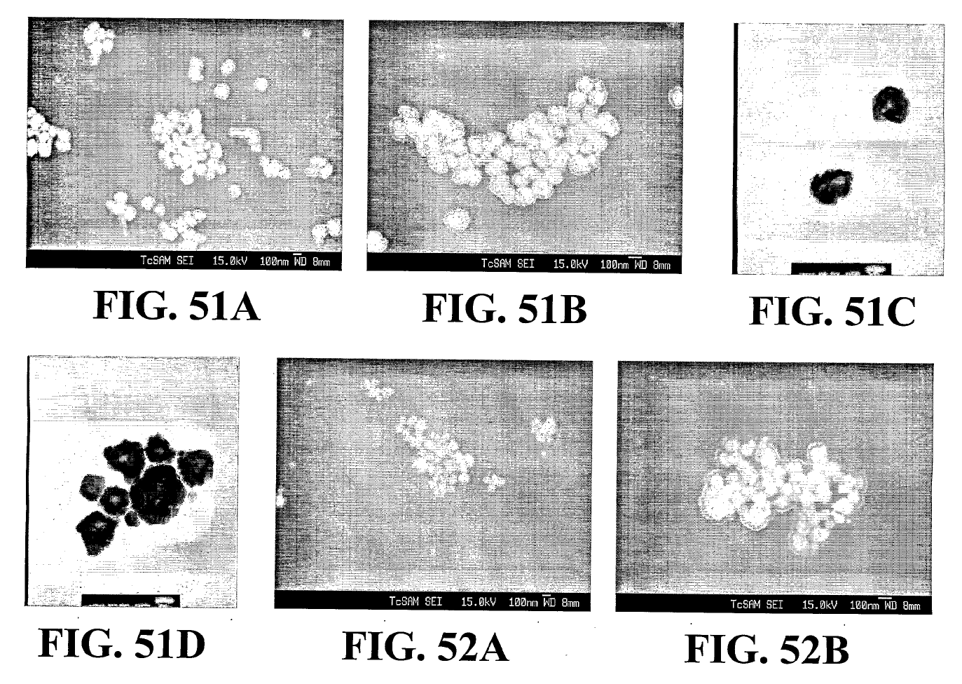

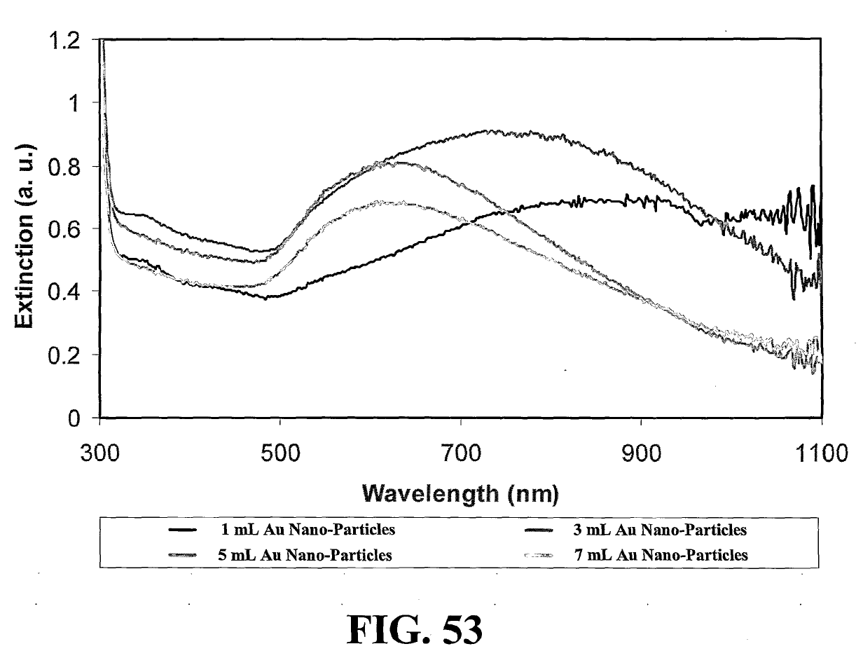

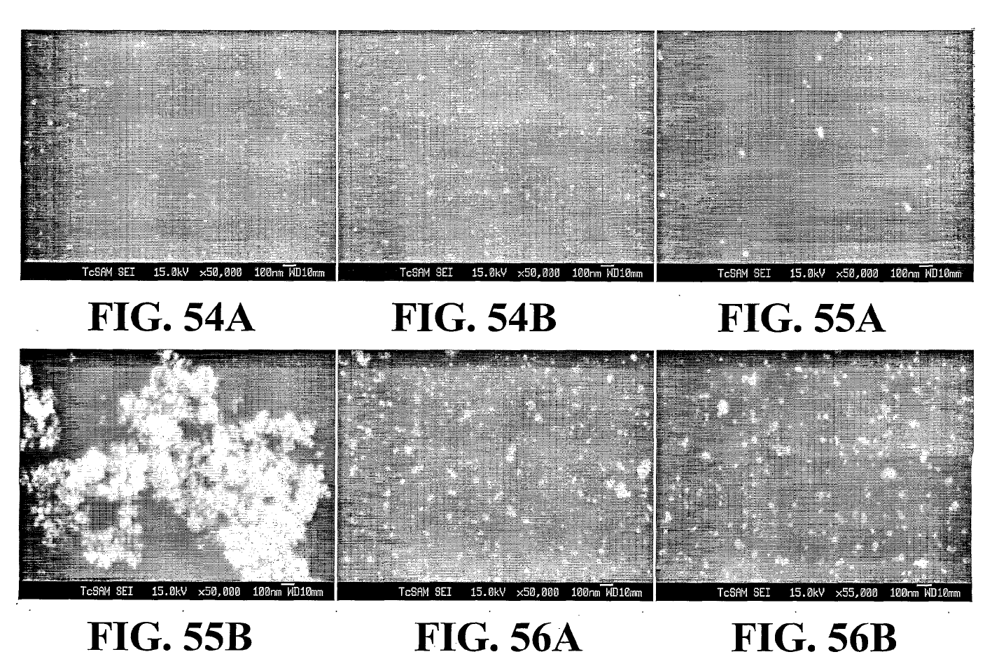

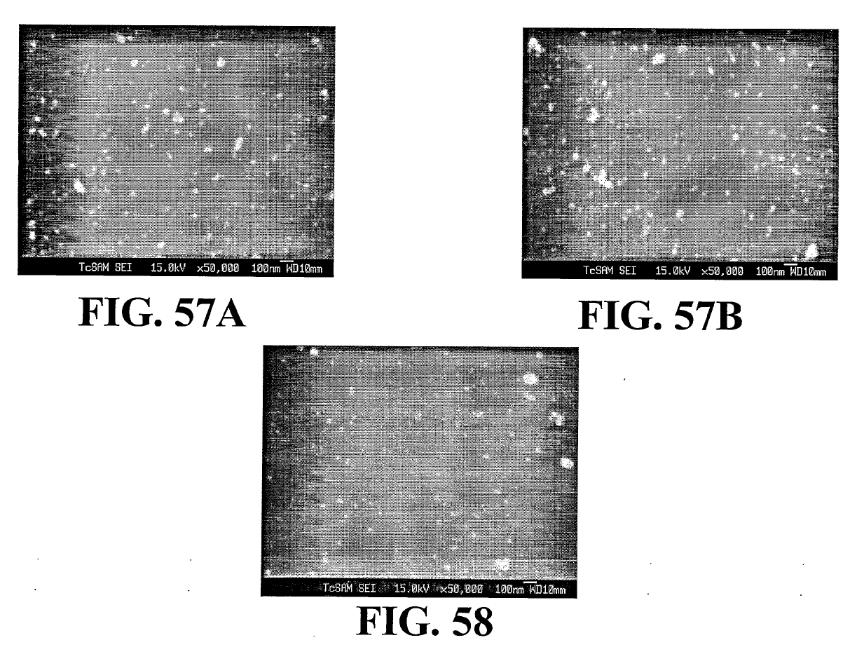

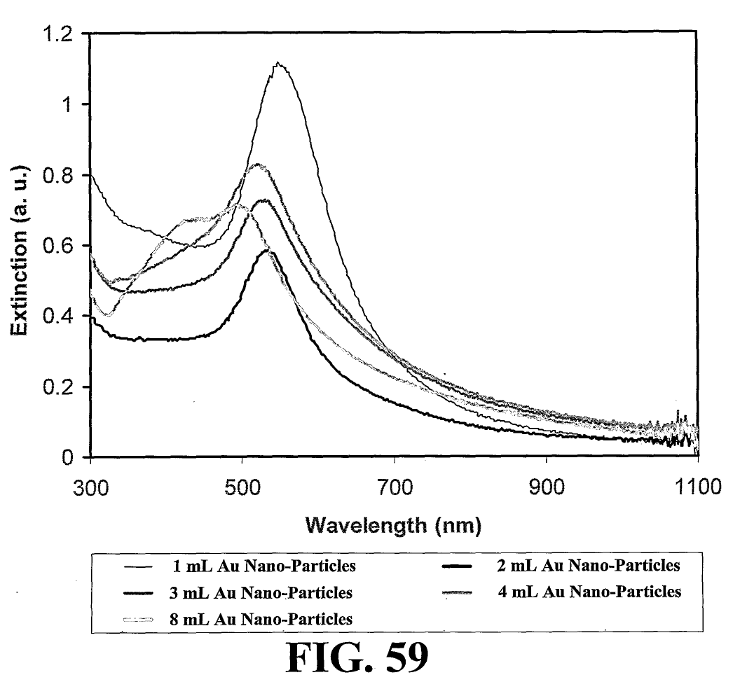

Referring now to FIGS. 26A&B, SEM images of 50-60 nm gold nano-particles. FIGS. 27A&B show FE-SEM images of 50-60 nm gold nano-particles coated with a gold nano-shell where 7 mL of gold nano-particle solution were used in the above preparation. FIGS. 28A&B show FE-SEM images of 50-60 nm gold nano-particles coated with a gold nano-shell where 3 mL of gold nano-particle solution were used in the above preparation. FIGS. 29 shows WV-vis spectra of 50-60 nm gold nano-particles with nano-shells prepared with 1 mL, 3 mL, 5 mL, and 7 mL of the 50-60 nm gold nano-particle solution according to the above preparation. FIGS. 30A&B show FE-SEM images of 50-60 nm gold nano-particles. FIGS. 31A&B show FE-SEM images of 50-60 nm gold nano-particles coated with a gold nano-shell where 3 mL of gold nano-particle solution and a low concentration of reducing agent were used in the above preparation. FIGS. 32A-C show FE-SEM images of 50-60 nm gold nano-particles coated with a gold nano-shell where 5 mL of gold nano-particle solution and a low concentration of reducing agent were used in the above preparation. FIGS. 33A&B show FE-SEM images of 50-60 nm gold nano-particles coated with a gold nano-shell where 7 mL of gold nano-particle solution and a low concentration of reducing agent were used in the above preparation. FIG. 34 shows UV-vis spectra of 50-60 nm gold nano-particles with nano-shells prepared with 3 mL, 5 mL, and 7 mL of the 50-60 nm gold nano-particle solution in the above preparation. FIGS. 35A&B show FE-SEM images of 10-15 nm gold nano-particles. FIGS. 36A-C show FE-SEM images of 10-15 nm gold nano-particles coated with a gold nano-shell where 1 mL of gold nano-particle solution were used in the above preparation. FIGS. 37A&B show FE-SEM images of 10-15 nm gold nano-particles coated with a gold nano-shell where 3 mL of gold nano-particle solution were used in the above preparation. FIGS. 38A-C show FE-SEM images of 10-15 nm gold nano-particles coated with a gold nano-shell where 5 mL of gold nano-particles were used in the above preparation. FIGS. 39A&B show FE-SEM images of 10-15 nm gold nano-particles coated with a gold nano-shell where 9 mL of gold nano-particle solution were used in the above preparation. FIGS. 40A&B show FE-SEM images of 10-15 nm gold nano-particles coated with a gold nano-shell where 2 mL of gold nano-particle solution were used in the above preparation. FIGS. 41A&B show FE-SEM images of 10-15 nm gold nano-particles coated with a gold nano-shell where 6 mL of gold nano-particle solution were used in the above preparation. FIGS. 42A&B show FE-SEM images of 10-15 nm gold nano-particles coated with a gold nano-shell where 11 mL of gold nano-particle solution were used in the above preparation. FIG. 43 shows UV-vis spectra of 10-15 nm gold nano-particles and nano-shells nano-particles prepared with 1 mL, 2 mL, 3 mL, 5 mL, 6 mL, 7 mL, 9 mL and 11 mL of the 10-15 nm gold nano-particle solution in the above preparation. FIGS. 44A&B show FE-SEM images of 50-60 nm silver nano-particles. FIG. 45 shows an FE-SEM images of 50-60 nm silver nano-particles coated with a gold nano-shell where 1 mL of silver nano-particle solution were used in the above preparation. FIG. 46 shows an FE-SEM images of 50-60 nm silver nano-particles coated with a gold nano-shell where 3 mL of silver nano-particle solution were used in the above preparation. FIG. 47 shows an FE-SEM images of 50-60 nm silver nano-particles coated with a gold nano-shell where 7 mL of silver nano-particle solution were used in the above preparation. FIG. 48 shows UV-vis spectra of 50-60 nm silver nano-particles and nano-shells nano-particles prepared with 1 mL, 3 mL, 5 mL, and 7 mL of the 50-60 nm silver nano-particle solution in the above preparation. FIGS. 49A-C show FE-SEM images of 50-60 nm silver nano-particles coated with a gold nano-shell where 1 mL of silver nano-particle solution and a low concentration of reducing agent were used in the above preparation. FIGS. 50A&B show FE-SEM images of 50-60 nm silver nano-particles coated with a gold nano-shell where 3 mL of silver nano-particle solution and a low concentration of reducing agent were used in the above preparation. FIGS. 51A-D show FE-SEM images of 50-60 nm silver nano-particles coated with a gold nano-shell where 5 mL of silver nano-particle solution and a low concentration of reducing agent were used in the above preparation. FIGS. 52A&B show FE-SEM images of 50-60 nm silver nano-particles coated with a gold nano-shell where 7 mL of silver nano-particle solution were used in the above preparation. FIG. 53 shows UV-vis spectra of 50-60 nm nano-shells nano-particles prepared with 1 mL, 3 mL, 5 mL, and 7 mL of the 50-60 nm silver nano-particle solution in the above preparation. FIGS. 54A&B show FE-SEM images of 10-15 nm silver nano-particles. FIGS. 55A-C show FE-SEM images of 10-15 nm silver nano-particles coated with a gold nano-shell where 1 mL of gold nano-particle solution were used in the above preparation. FIGS. 56A&B show FE-SEM images of 10-15 nm silver nano-particles coated with a gold nano-shell where 2 mL of gold nano-particle solution were used in the above preparation. FIGS. 57A-C show FE-SEM images of 10-15 nm silver nano-particles coated with a gold nano-shell where 3 mL of gold nano-particle solution were used in the preparation. FIG. 58 shows an FE-SEM image of 10-15 nm silver nano-particles coated with a gold nano-shell where 4 mL of the gold nano-particle solution were used in the above preparation. FIG. 59 shows UV-vis spectra of 10-15 nm nano-shells nano-particles prepared with 1 mL, 2 mL, 3 mL, 4 mL, and 8 mL of the 10-15 nm silver nano-particle solution in the above preparation.

### EXAMPLE 8

### Gold Nano-Rods on Silver Nano-Particles

Materials

Sodium citrate dihydrate, nitric acid, hydrochloric acid (EM Science), hydrogen teterachloroaurate-(III) hydrate (Strem), terakis(hydroxoymethyl)phosphonium chloride (THPC, Aldrich), silver nitrate (Mallinckrodt), and L-ascorbic acid from Chemalog were purchased from indicated companies. All the chemicals were used as received without purification. Water was purified to a resistance of 18 MΩ (Academic Milli-Q Water System; Millipore Corporation) and filtered using 0.22 μm membrane filter. All glassware used in the experiment was cleaned in a strong acid and base prior to use.

Preparation of Silver Nano-particle Cores

is a slight modification of well known Lee and Meisel method to make variable sizes of silver nanoparticles (over 40 nm in diameter). (Langmuir 2001, 17. 574-577. Journal of Colloid and Interface and Science, 1983, 93, 545-555. J. Phys. Chem. 1982, 86, 3991.) 200 ml of a 10⁻³ M AgNO₃ solution was heated to boiling, and added 4 ml of a 1% trisodium citrate as soon as it reaches boiling. The mixture was kept stirring and boiling for 45 min to get homogeneous silver nanoparticles (˜60 nm). The solution was stirred for 5 more min and aged for 2 hr before use.

Gold Nano-rods Grown on Silver Nano-particle Cores

To grow the gold nanorods on the silver nanopartcle cores, 5 ml of HAuCl₄.H₂O (1 mM) was mixed with 0.2 mL of AgNO₃ (4 mM and 1 ml of prepared silver nanoparticles. 0.07 ML of L-ascorbic acid (78.8 mM) was added to the mixture and the final solution was shaken for a couple of minutes to react. The color change occurred from light yellow to green and blue within 5 minutes dependent on the amount of the silver nanoparticle solution. The mixture was centrifuged at 2500 rpm for 1 h using RC-3B Refrigerated Centrifuge (Sorvall Instruments) and redispersed in Milli-Q water for the analysis.

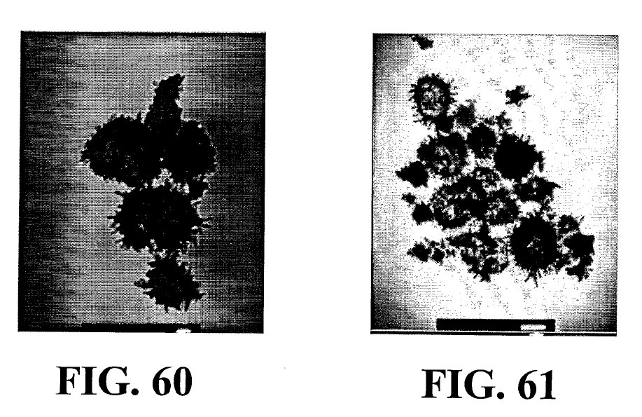

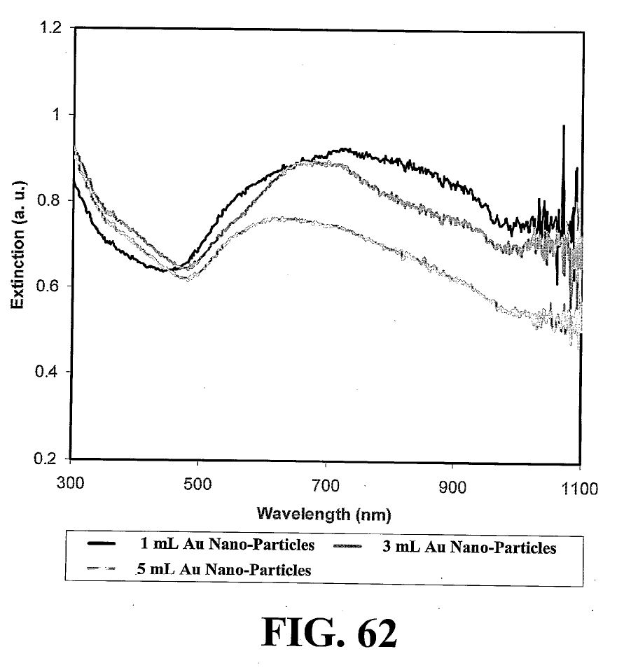

Referring now to FIG. 60, an FE-SEM image of a 50-60 nm silver nano-particles having gold nano-rods formed thereon to form a sweet gum ball type structure where 1 mL of the silver nano-particle solution. FIG. 61 shows an FE-SEM image of a 50-60 nm silver nano-particles having gold nano-rods formed thereon to form a sweet gum ball type structure where 3 mL of the silver nano-particle solution. FIG. 62 shows UV-vis spectra of 50-60 nm nano-shells nano-particles prepared with 1 mL, 3 mL, and 5 mL the 50-60 nm silver nano-particle solution. Although the synthesis is described using silver nano-particles, the same synthesis will work for gold nano-particles, metal nano-shell dielectric nano-particles, metal nano-shell metal nano-particles and mixtures or combinations thereof.

### EXAMPLE 9

### Gold Nano-Particle Growth in Hydrogel Polymer Nano-Particles

Materials

N-isopropylacrylamide (NIPAM) (99% purity from Acros), recrystallized in hexane, and dried under vacuum before use. Sodium dodecyl sulfate (SDS) (from Promega), N,N′-methylenebisacrylamide (BIS), Acrylic acid (AAc) (from Acros), terakis(hydroxoymethyl)phosphonium chloride (THPC) (from Aldrich), hydrogen tetrachloroaurate (Au 99.9%) (from Strem), potassium hydroxide, nitric acid, and ammonium persulfate (APS) (from EM Science) were all used as received from the indicated suppliers. Water used in all reactions, solution preparations, and polymer isolations was purified to a resistance of 18 MΩ using an Academic Milli-Q Water System from Millipore Corporation and filtered through 0.22 μm filter membrane to remove any impurities (Milli-Q water).

Synthesis of Hydrogel Nano-Particles

Different sizes of hydrogel nanoparticles were prepared by emulsion polymerzation in aqueous solution. (Clinton et al. Macromolecules 2000, 33, 8301. Martin et. al. J. Chem. Soc. Faraday Transaction 1996, 92, 5013.)

In a three-necked round-bottomed flask equipped with a reflux condenser and an inlet for argon gas, NIPAM (1 g), AAc (0.05 g), and BIS (0.1 g) were dissolved in 196 mL of purified Milli-Q water. The solution was purged with argon for 1 h and argon was bubbled through the solution for the duration of the reaction to remove any oxygen, which can intercept radicals and disrupt the polymerization. The solution was agitated using a football-shaped Teflon-coated magnetic stirring bar. The solution was then heated to 71° C. in an oil bath, and then APS (0.4 g/4 mL Milli-Q water) was added to initiate the polymerization. The reaction time, which depended on the amount of starting materials, was varied between 5 and 6 h. At the end of this period, the solution was cooled and filtered through a 1 μm membrane to remove any micron-sized impurities and/or any aggregated particles. The size of the hydrogel nano-particles was controlled by the amount of monomer and initiator as well as the reaction time.

Gold Nano-Particle Growth to Form Hydrogel-Coated Gold Nano-Particles

To produce gold nano-particles coated with hydrogel polymer, tetrakis(hydroxymethyl)phosphonium chloride (THPC) was used as the gold reducing agent as a modification of the Duff et al method. (See, e.g., Duff, D. G.; Baiker, A. Langinuir 1993, 9, 2301. Duff, D. G.; Baiker, A. Langmuir 1993, 9, 2310.) 50 mL of prepared hydrogel nano-particles were mixed with 1.88 mL of an aqueous hydrogen teterachloroaurate(III) hydrate solution (1 wt. %, HAuCl₄.3H₂O) and stirred for at least 30 min. 0.33 mL of 1 M sodium hydroxide (NaOH), 1.11 mL of a THPC solution comprising 12 μL of a 80 wt. % solution of THPC in 1 mL of Milli-Q water were added to the mixture at the same time with stirring. A solution color change occurred quickly from colorless to pink, red, and brown in a few minutes, and the solution was stirred for another 30 min. The final solution was centrifuged at 30° C. for 2 h at 3500 rpm using a RC-3B Refrigerated Centrifuge (Sorvall Instruments), and the supernatant was separated to remove unreacted materials, soluble side products, small gold seed particles, and seeds of pure polymer. The purified nano-particles were then diluted with pure Milli-Q water and stored at room temperature for later use. The size of the hydrogel-coated gold particles was same as bare hydrogel nano-particles and the size of the gold core nano-particles was controlled by the amount of gold salt, sodium hydroxide and the THPC in the preparation solution.

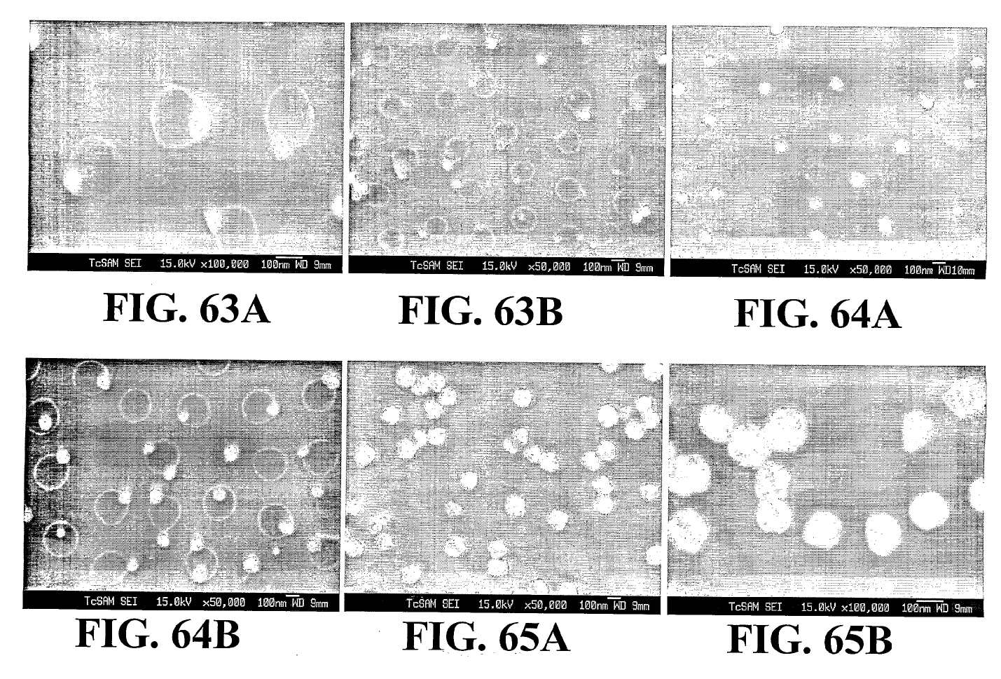

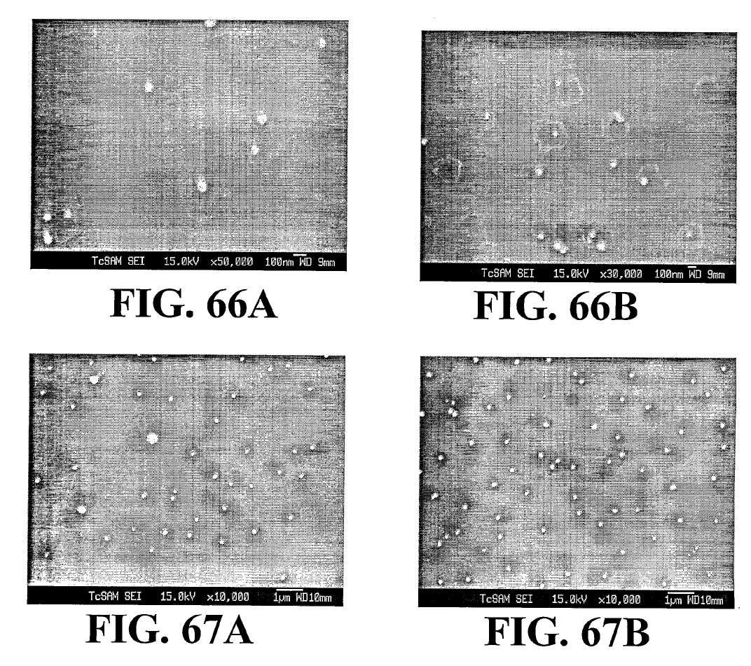

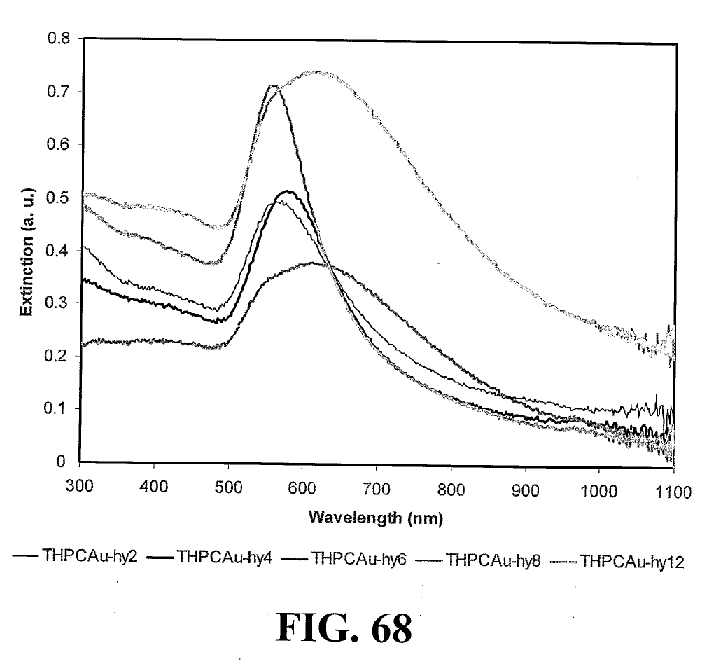

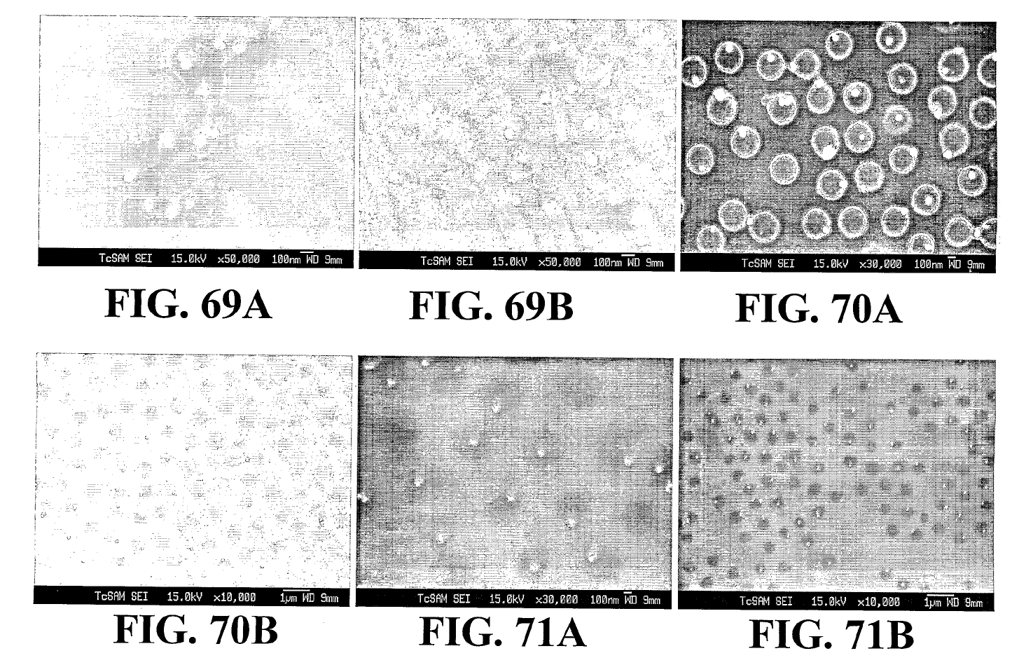

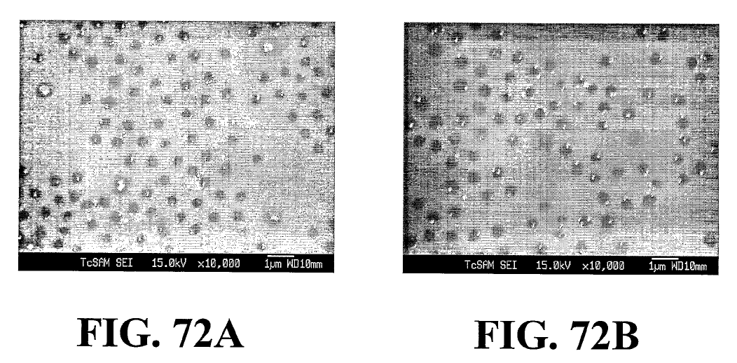

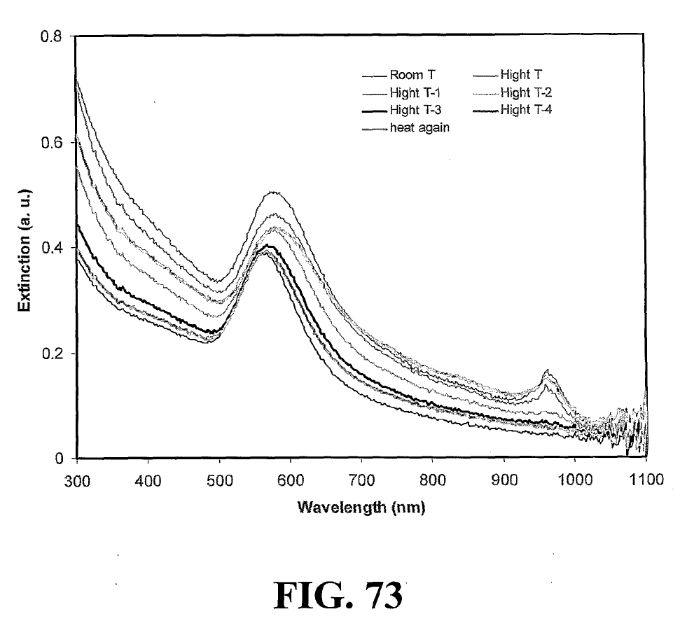

Referring now to FIGS. 63A&B, FE-SEM images of 200 nm hydrogel nano-particles of a homopolymerNIPAM with 50 nm gold nano-particles grown therein are shown. FIGS. 64A&B show FE-SEM images of a 500 nm hydrogel nano-particles of a homopolymer NIPAM with 80 nm gold nano-particles grown therein. FIGS. 65A&B show FE-SEM images of large ˜100 nm gold nano-particles. FIGS. 66A&B show FE-SEM images of a 500 nm hydrogel nano-particles of a co-polymer of acrylic acid and NIPAM having 40 nm gold nano-particles grown therein. FIGS. 67A&B show FE-SEM images of a 500 nm hydrogel nano-particles of a co-polymer of acrylic acid and NIPAM having 100 nm gold nano-particles grown therein. FIG. 68 depicts UV-vis spectra of nano-particles of FIGS. 59-63. FIGS. 69A&B show FE-SEM images of 500 nm hydrogel nano-particles of a homopolymer NIPAM with 80 nm gold nano-particles grown therein treated at high temperature before imaging. FIGS. 70A&B show FE-SEM images of a 500 nm hydrogel nano-particles of a homopolymer NIPAM with 80 nm gold nano-particles grown therein dried at room temperature with regular imaging. FIGS. 71A&B show FE-SEM images of a 500 nm hydrogel nano-particles of a homopolymer NIPAM with 80 nm gold nano-particles grown therein dried at 30° C. under vacuum at 24 hours showing that the hydrogel collapsed to a diameter of 400 nm. FIGS. 72A&B show FE-SEM images of a 500 nm hydrogel nano-particles of a homopolymer NIPAM with 80 nm gold nano-particles grown therein dried at 80° C. for 4 hours showing that the hydrogel collapsed to a diameter of 400 nm. FIG. 73 shows UV-vis spectra of nano-particles of FIGS. 65-68.

### REFERENCES CITED IN THE INVENTION

The following references are have been cited in the specification above:

1. A. S. Hoffinan et al. J. Contr. Rel. 4:213-222, 1986.

2. L. C. Dong et al. J. Contr. Rel. 4:223-227, 1986.

3. R. Yoshida et al. J. Biomater. Sci. Polymer Edn. 6:585-598, 1994.

4. Stober, W.; Fink, A.; Bohn, E. J. Colloid Interface Sci. 1968, 26, 62.

5. Waddell, T. G.; Leyden, D. E.; DeBello, M. T. J. Am. Chem. Soc. 1981, 103, 5303.

6. van Blaaderen, A.; Vrij, A. J. J. Colloid Interface Sci. 1993, 156, 1.

7. Duff, D. G.; Baiker, A. Langmuir 1993, 9, 2301.

8. Duff, D. G.; Baiker, A. Langmuir 1993, 9, 2310.

9. Lee and Meisel, Langmuir 2001, 17, 574.

10. Lee and Meisel, Journal of Colloid and Interface and Science 1983, 93, 545.

11. Lee and Meisel, J. Phys. Chem. 1982, 86, 3991.

12. Stober, W.; Fink, A.; Bohn, E. J. Colloid Interface Sci. 1968, 26, 62.

13. Nikhil R. Jana, Latha Gearheart and Catherine J. Murphy Chem. Commun. 2001, 617.

14. Nikhil R. Jana, Latha Gearheart, Catherine J. Murphy Adv. Mater. 2001, 13, 1389.

15. Stober, W.; Fink, A.; Bohn, E. J. Colloid Interface Sci. 1968, 26, 62.

16. Teo, B. K.; Keating, K.; Kao, Y-H. J. Am. Chem. Soc. 1987, 109, 3494.

17. Westscott, S. L.; Oldenburg, S. J.; Lee, T. R.; Halas, N. J. Langmuir 1998, 14, 5396.

18. T. Pham; J. B. Jackson; N. J. Halas; T. R. Lee Langmuir 2002, 18, 4915.

19. Quanroni, L.; Chumanov, G. J. Am. Chem. Soc. 1999, 121, 10642.

20. Clark, H. A.; Campagnok, P. J.; Wuskell, J. P.; Lewis, A.; Loew, L. M. J. Am. Chem. Soc. 2000, 122, 10234.

All references cited herein are incorporated by reference. While this invention has been described fully and completely, it should be understood that, within the scope of the appended claims, the invention may be practiced otherwise than as specifically described. Although the invention has been disclosed with reference to its preferred embodiments, from reading this description those of skill in the art may appreciate changes and modification that maybe made which do not depart from the scope and spirit of the invention as described above and claimed hereafter.

1. A composition comprising a nano-particle core and a nano-structure formed an outer surface of the core, where the nano-particle core comprises a first conductive material and the structure comprises a second conductive material, where the first and second conductive materials are the same or different.

2. (canceled)

3. (canceled)

4. (canceled)

5. (canceled)

6. (canceled)

7. (canceled)

8. (canceled)

9. (canceled)

10. (canceled)

11. A nano-structure compositioncomprising anano-particle coreand aplurality of nano-rods, where the nano-particle core comprises a first material and the nano-rods comprises a first conductive material.

12. The composition of claim 11, further comprising a nano-shell interposed between the core and the nano-rods where the nano-shell comprises a second conductive material, where the first and second conductive materials are the same or different.

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. The composition of claim 1, wherein the nano-structure is selected from the group consisting of a nano-shell, a plurality of nano-rods and a nano-shell having a plurality of nano-rods disposed on a surface of the nano-shell, where the nano-rods comprise a third conductive material, where the first, second and third conductive materials are the same or different.

32. The composition of claim 1, wherein the first conductive material comprises a first metal, metal alloy or a conductive polymer and the second conductive material comprises a second metal or metal alloy.

33. The composition ofclaim 31, wherein the first conductive material comprises a first metal, metal alloy or a conductive polymer, the second conductive material comprises a second metal or metal alloy, and the third conductive material comprises third metal or metal alloy, where the first, second and third metals and/or metal alloys are the same or different.

34. The composition of claim 32, wherein the first, second and third metals or metal alloys are the same or different noble metals or metal alloys, where the noble metal are selected from the group consisting of gold, silver, platinum, palladium, iridium, osmium, ruthenium, rhodium, and mixtures or combinations thereof.

35. The composition of claim 33, wherein the first, second and third metals or metal alloys are the same or different noble metals or metal alloys, where the noble metal are selected from the group consisting of gold, silver, platinum, palladium, iridium, osmium, ruthenium, rhodium, and mixtures or combinations thereof.

36. The composition of 1, wherein the first metal and first metal alloy are selected respectively from the group consisting of non-transition metals, non-transition metal alloys, transition metals, transition metal alloys, lanthanide metals, lanthanide metal alloys, actinide metals, and actinide metal alloys.

37. The composition of 31, wherein the first metal and first metal alloy are selected respectively from the group consisting of non-transition metals, non-transition metal alloys, transition metals, transition metal alloys, lanthanide metals, lanthanide metal alloys, actinide metals, and actinide metal alloys.

38. The composition of 1, wherein the nano-structure has a plasmon resonance having a frequency range at least a portion of which lies in the near infrared region of the electromagnetic spectrum.

39. The composition of 31, wherein the nano-structure has a plasmon resonance having a frequency range at least a portion of which lies in the near infrared region of the electromagnetic spectrum.

40. A nano-structure composition comprising a nano-particle core, a nano-structure formed an outer surface of the core and a bio-compatible polymer coating the structure and the core, where the nano-structure is selected from the group consisting of a nano-shell, a plurality of nano-rods and a nano-shell having a plurality of nano-rods disposed on a surface of the nano-shell, where the nano-particle core comprises a first material, the nano-shell comprises a second conductive material, and the nano-rods comprise a third conductive material, where the second and third conductive materials are the same or different.

41. The composition of claim 40, wherein the first material is a non-conductive material, a semi-conductor material or a conductive material.

42. The composition ofclaim 41, wherein the first conductive material comprises a first metal, metal alloy or a conductive polymer, the second conductive material comprises a second metal or metal alloy, and the third conductive material comprises third metal or metal alloy, where the first, second and third metals and/or metal alloys are the same or different.

43. The composition of claim 42, wherein the first, second and third metals or metal alloys are the same or different noble metals or metal alloys, where the noble metal are selected from the group consisting of gold, silver, platinum, palladium, iridium, osmium, ruthenium, rhodium, and mixtures or combinations thereof.

44. The composition of 41, wherein the first metal and first metal alloy are selected respectively from the group consisting of non-transition metals, non-transition metal alloys, transition metals, transition metal alloys, lanthanide metals, lanthanide metal alloys, actinide metals, and actinide metal alloys.

45. The composition of claim 40, wherein the nano-structure has a plasmon resonance having a frequency range at least a portion of which lies in the near infrared region of the electromagnetic spectrum.

46. A nano-structure drug-delivery composition comprising a nano-particle core, a nano-structure, a bio-compatible polymer coating and a pharmaceutically active agent impregnated into the polymer coating, where the nano-structure is selected from the group consisting of a nano-shell, a plurality of nano-rods and a nano-shell having a plurality of nano-rods disposed on a surface of the nano-shell, where the nano-particle core comprises a first material, the nano-shell comprises a second conductive material, and the nano-rods comprise a third conductive material, where the second and third conductive materials are the same or different.

47. A nano-structure drug-delivery composition comprising a nano-particle core, a nano-structure formed on an outer surface of the core, and a pharmaceutically active agent absorbed or attached thereto, where the nano-structure is selected from the group consisting of a nano-shell, a plurality of nano-rods and a nano-shell having a plurality of nano-rods disposed on a surface of the nano-shell, where the nano-particle core comprises a first material, the nano-shell comprises a second conductive material, and the nano-rods comprise a third conductive material, where the second and third conductive materials are the same or different.

48. A method for treating cancers or diseases comprising:
administering a composition to an animal including a human and
exposing the composition to an electromagnetic, magnetic, electrical and/or ultrasonic field so that the nano-structures convert the field into thermal energy,
where the composition comprises a nano-particle core, a nano-structure formed an outer surface of the core and a bio-compatible polymer coating the structure and the core or a pharmaceutically active agent absorbed or attached thereto, where the nano-structure is selected from the group consisting of a nano-shell, a plurality of nano-rods and a nano-shell having a plurality of nano-rods disposed on a surface of the nano-shell, where the nano-particle core comprises a first material, the nano-shell comprises a second conductive material, and the nano-rods comprise a third conductive material, where the second and third conductive materials are the same or different.
